(19) United States
(12) Patent Application Publication
(10) Pub. No.: US 2026/0250269 A1
(43) Pub. Date: Aug. 27, 2026

(54) APOPTOSIS INHIBITOR SALTS FOR CANCER

(71) Applicants: Chun JIANG, Hillsborough, CA (US); Haiwen ZHANG, Beijing (CN)

(72) Inventors: Chun JIANG, Hillsborough, CA (US); Haiwen ZHANG, Beijing (CN)

(21) Appl. No.: 18/728,015

(22) PCT Filed: Jan. 11, 2023
(86) PCT No.: PCT/CN2023/071763

§ 371 Date: Jul. 10, 2024

(30) Foreign Application Priority Data
Jan. 11, 2022 (WO) PCT/CN2022/071439

(51) Int. Cl. C07D 403/06; A61K 31/337; A61K 31/497; A61K 31/555; A61K 31/573; A61P 35/00; C07C 65/11; C07K 16/28

(52) U.S. Cl. CPC C07D 403/06; A61K 31/337; A61K 31/497; A61K 31/555; A61K 31/573; A61P 35/00; C07C 65/11; C07K 16/2887

(57) ABSTRACT

It relates to 1-(2-methoxyethyl)-2-methyl-4, 9-dioxo-3-(pyrazin-2-ylmethyl)-4, 9-dihydro-1H-naphtho [2, 3-d]imidazolium pamoate or solvate thereof, pharmaceutical composition comprising the pamoate salt or solvate, and therapeutic uses thereof. In particular, the pamoate salt or solvate is useful for treating cancer which has a c-My rearrangement and/or MYCN amplification or overexpression.

### FIELD OF THE INVENTION

The present disclosure relates to 1-(2-methoxyethyl)-2-methyl-4,9-dioxo-3-(pyrazin-2-ylmethyl)-4,9-dihydro-1H-naphtho[2,3-d]imidazolium pamoate or solvate thereof, pharmaceutical composition comprising the pamoate salt or solvate, and therapeutic uses thereof. In particular, the pamoate salt or solvate is useful for treating cancer which has a c-Myc rearrangement and/or MYCN amplification or overexpression.

### BACKGROUND OF THE INVENTION

Sepantronium bromide (1-(2-methoxyethyl)-2-methyl-4,9-dioxo-3-(pyrazin-2-ylmethyl)-4,9-dihydro-1H-naphtho[2,3-d]imidazolium bromide), also known as YM155 monobromide, or YM155, is a small imidazolium-based proapoptotic agent, which is being developed for treating solid tumors and hematologic malignancies, including B-cell lymphomas and prostate cancer.

Sepantronium bromide selectively suppresses survivin mRNA and protein expression in vitro and induces tumor cell death in a dose-dependent manner. The antiproliferative effect of sepantronium bromide in vitro is potentiated by longer duration of exposure and is therefore time dependent. Currently, sepantroinum bromide is being studied for a continuous IV infusion administration for 7 days in a 21-day treatment cycle.

Continuous infusion over multiple days is not an ideal route of administration. Thus, there is a need to provide sepantroium bromide or its analog or a derivative in a form would not require continuous infusion yet provides the therapeutic efficacy required for treatment.

### SUMMARY OF THE INVENTION

The present disclosure provides a pamoate salt, 1-(2-methoxyethyl)-2-methyl-4,9-dioxo-3-(pyrazin-2-ylmethyl)-4,9-dihydro-1H-naphtho[2,3-d]imidazolium pamoate, or a solvate thereof. In one embodiment, the pamoate salt is an anhydrate. In some embodiments, the pamoate salt is a solvate.

In some embodiments of the pamoate salt or solvate thereof, the solvate is an ethanol solvate, an N-methyl pyrrolidone solvate, an N,N-dimethylacetamide solvate, a hexafluoroisopropyl acrylate solvate, or an N,N-dimethylformamide solvate.

In some embodiments of the pamoate salt or solvate thereof, the pamoate salt or the solvate is crystalline. In some embodiments, the pamoate salt or the solvate is amorphous.

In some embodiments of the pamoate salt or solvate thereof, the pamoate salt or the solvate has a purity of about 90% or higher. In some embodiments, the pamoate salt or the solvate has a purity of about 95% or higher. In some embodiments, the pamoate salt or the solvate has a purity of about 98% or higher.

In some embodiments of the pamoate salt or solvate thereof, the pamoate salt comprises less than 0.5% of bromide ion (Br⁻) by weight.

In some embodiments of the pamoate salt or solvate thereof, the pamoate salt or solvate thereof is purified and isolated.

The present disclosure further provides a pharmaceutical composition comprising any one of the pamoate salt as disclosed herein and a pharmaceutically acceptable excipient or carrier. In some embodiments, the pharmaceutical composition comprises the pamoate salt comprising less than 0.5% of bromide ion (Br⁻) by weight of the pamoate salt. In some embodiments, the pharmaceutical composition comprises the pamoate salt comprising less than 0.4% of bromide ion (Br⁻) by weight of the pamoate salt. In some embodiments, the pharmaceutical composition comprises the pamoate salt comprising less than 0.3% of bromide ion (Br⁻) by weight of the pamoate salt. In some embodiments, the pharmaceutical composition comprises the pamoate salt comprising less than 0.2% of bromide ion (Br⁻) by weight of the pamoate salt. In some embodiments, the pharmaceutical composition comprises the pamoate salt comprising less than 0.1% of bromide ion (Br⁻) by weight of the pamoate salt.

The present disclosure further provides a crystalline form of a pamoate salt or a solvate thereof, wherein the pamoate salt is 1-(2-methoxyethyl)-2-methyl-4,9-dioxo-3-(pyrazin-2-ylmethyl)-4,9-dihydro-1H-naphtho[2,3-d]imidazolium pamoate.

In some embodiments of the crystalline form of the pamoate salt or solvate thereof, the pamoate salt is an anhydrate.

In some embodiments, the pamoate salt anhydrate exhibits an X-ray powder diffraction (XRPD) pattern comprising peaks at about 8.08±0.2, 9.69±0.2, and 10.67±0.2 degrees two-theta. In some embodiments, the XRPD pattern further comprises peaks at 11.41±0.2, 15.29±0.2, and 16.17±0.2 degrees two-theta. In some embodiments, the pamoate salt anhydrate exhibits an XRPD pattern substantially similar to FIG. 1A. In some embodiments, the pamoate salt anhydrate exhibits a differential scanning calorimetry (DSC) thermogram comprising an endotherm peak which onsets at about 248° C. In some embodiments, the pamoate salt anhydrate exhibits weight percent loss of about 0.8% between about 35° C. to about 230° C. by a thermogravimetric analysis (TGA).

In some embodiments, the pamoate salt anhydrate exhibits an X-ray powder diffraction (XRPD) pattern comprising peaks at about 9.32±0.2 and 16.89±0.2 degrees two-theta. In some embodiments, the XRPD pattern further comprises peaks at 10.97±0.2, 11.60±0.2, and 24.77±0.2 degrees two-theta. In some embodiments, the pamoate salt anhydrate exhibits an XRPD pattern substantially similar to FIG. 2A. In some embodiments, the pamoate salt anhydrate exhibits a differential scanning calorimetry (DSC) thermogram comprising an endotherm peak which onsets at about 248° C. In some embodiments, the pamoate salt anhydrate exhibits weight percent loss of about 0.6% between about 35° C. to about 220° C. by a thermogravimetric analysis (TGA).

In some embodiments of the crystalline form of the pamoate salt or solvate thereof, the pamoate salt is a solvate.

In some embodiments of the crystalline form of the pamoate salt or solvate thereof, the pamoate salt is an ethanol solvate. In some embodiments, the pamoate salt ethanol solvate exhibits an X-ray powder diffraction (XRPD) pattern comprising peaks at about 7.39±0.2 and 26.03±0.2 degrees two-theta. In some embodiments, the XRPD pattern further comprises peaks at 10.12±0.2, 21.26±0.2, and 25.70±0.2 degrees two-theta. In some embodiments, the pamoate salt ethanol solvate exhibits an XRPD pattern substantially similar to FIG. 3A. In some embodiments, the pamoate salt ethanol solvate exhibits a differential scanning calorimetry (DSC) thermogram comprising an endotherm peak which onsets at about 238° C. In some embodiments, the pamoate salt ethanol solvate exhibits a differential scanning calorimetry (DSC) thermogram comprising an exotherm peak which onsets at about 170° C. In some embodiments, the pamoate salt ethanol solvate exhibits weight percent loss of about 7.2% between about 35° C. to about 200° C. by a thermogravimetric analysis (TGA).

In some embodiments of the crystalline form of the pamoate salt or solvate thereof, the pamoate salt is an N-methyl pyrrolidone solvate. In some embodiments, the pamoate salt N-methyl pyrrolidone solvate exhibits an X-ray powder diffraction (XRPD) pattern comprising peaks at about 7.46±0.2, 20.27±0.2, and 24.93±0.2 degrees two-theta. In some embodiments, the XRPD pattern further comprises peaks at 21.05±0.2, 22.44±0.2, and 24.57±0.2 degrees two-theta. In some embodiments, the pamoate salt N-methyl pyrrolidone solvate exhibits an XRPD pattern substantially similar to FIG. 4A. In some embodiments, the pamoate salt N-methyl pyrrolidone solvate exhibits a differential scanning calorimetry (DSC) thermogram comprising an endotherm peak which onsets at about 243° C. In some embodiments, the pamoate salt N-methyl pyrrolidone solvate exhibits weight percent loss of about 33% between about 35° C. to about 140° C. by a thermogravimetric analysis (TGA). In some embodiments, the pamoate salt N-methyl pyrrolidone solvate exhibits weight percent loss of about 90.5% between about 140° C. to about 220° C. by a thermogravimetric analysis (TGA).

In some embodiments of the crystalline form of the pamoate salt or solvate thereof, the pamoate salt is an N,N-dimethylacetamide solvate. In some embodiments, the pamoate salt N,N-dimethylacetamide solvate exhibits an X-ray powder diffraction (XRPD) pattern comprising peaks at about 6.81±0.2, 8.31±0.2, and 24.60±0.2 degrees two-theta. In some embodiments, the pamoate salt N,N-dimethylacetamide solvate exhibits an XRPD pattern substantially similar to FIG. 5. In some embodiments, the pamoate salt N,N-dimethylacetamide solvate exhibits an X-ray powder diffraction (XRPD) pattern comprising peaks at about 11.44±0.2, 13.81±0.2, and 24.55±0.2 degrees two-theta. In some embodiments, the pamoate salt N,N-dimethylacetamide solvate exhibits an XRPD pattern further comprises peaks at 6.91±0.2, 12.21±0.2, and 22.70±0.2 degrees two-theta. In some embodiments, the pamoate salt N,N-dimethylacetamide solvate exhibits an XRPD pattern substantially similar to FIG. 6.

In some embodiments, the pamoate salt N,N-dimethylacetamide solvate exhibits an X-ray powder diffraction (XRPD) pattern comprising peaks at about 18.86 and 23.51 degrees two-theta. In some embodiments, the pamoate salt N,N-dimethylacetamide solvate exhibits an XRPD pattern further comprises peaks at 17.38±0.2, 20.54±0.2, and 21.24±0.2 degrees two-theta. In some embodiments, the pamoate salt N,N-dimethylacetamide solvate exhibits an XRPD pattern substantially similar to FIG. 7.

In some embodiments, the pamoate salt N,N-dimethylacetamide solvate exhibits an X-ray powder diffraction (XRPD) pattern comprising peaks at about 14.06±0.2 and 21.14±0.2 degrees two-theta. In some embodiments, the pamoate salt N,N-dimethylacetamide solvate exhibits an XRPD pattern further comprises peaks at 19.16±0.2, 21.93±0.2, and 22.36±0.2 degrees two-theta. In some embodiments, the pamoate salt N,N-dimethylacetamide solvate exhibits an XRPD pattern substantially similar to FIG. 8A. In some embodiments, the pamoate salt N,N-dimethylacetamide solvate exhibits a differential scanning calorimetry (DSC) thermogram comprising an endotherm peak which onsets at about 247° C. In some embodiments, the pamoate salt N,N-dimethylacetamide solvate exhibits weight percent loss of about 55% between about 35° C. to about 97° C. by a thermogravimetric analysis (TGA). In some embodiments, the pamoate salt N,N-dimethylacetamide solvate exhibits weight percent loss of about 5.3% between about 97° C. to about 125° C. by a thermogravimetric analysis (TGA).

In some embodiments of the crystalline form of the pamoate salt or solvate thereof, the pamoate salt is a hexafluoroisopropyl acrylate solvate. In some embodiments, the hexafluoroisopropyl acrylate solvate exhibits an X-ray powder diffraction (XRPD) pattern comprising peaks at about 4.73±0.2 and 9.45±0.2 degrees two-theta. In some embodiments, the hexafluoroisopropyl acrylate solvate exhibits an XRPD pattern substantially similar to FIG. 9A. In some embodiments, the hexafluoroisopropyl acrylate solvate exhibits a differential scanning calorimetry (DSC) thermogram comprising an endotherm peak which onsets at about 282° C. In some embodiments, the hexafluoroisopropyl acrylate solvate exhibits weight percent loss of about 2.3% between about 35° C. to about 80° C. by a thermogravimetric analysis (TGA). In some embodiments, the hexafluoroisopropyl acrylate solvate exhibits weight percent loss of about 8.0% between about 80° C. to about 200° C. by a thermogravimetric analysis (TGA).

In some embodiments of the crystalline form of the pamoate salt or solvate thereof, the pamoate salt is an N,N-dimethylformamide solvate. In some embodiments, the pamoate salt N,N-dimethylformamide solvate exhibits an X-ray powder diffraction (XRPD) pattern comprising peaks at about 5.82±0.2, 11.81±0.2, and 11.62±0.2 degrees two-theta. In some embodiments, the pamoate salt N,N-dimethylformamide solvate exhibits an XRPD pattern further comprises peaks at 17.46±0.2, 23.49±0.2, and 23.61±0.2 degrees two-theta. In some embodiments, the pamoate salt N,N-dimethylformamide solvate exhibits an XRPD pattern substantially similar to FIG. 10A. In some embodiments, the pamoate salt N,N-dimethylformamide solvate exhibits a differential scanning calorimetry (DSC) thermogram comprising an endotherm peak which onsets at about 249° C. In some embodiments, the pamoate salt N,N-dimethylformamide solvate exhibits weight percent loss of about 0.4% between about 35° C. to about 100° C. by a thermogravimetric analysis (TGA). In some embodiments, the pamoate salt N,N-dimethylformamide solvate exhibits weight percent loss of about 9.5% between about 100° C. to about 200° C. by a thermogravimetric analysis (TGA).

In some embodiments of the crystalline form of the pamoate salt or solvate thereof, the crystalline form has a purity of about 90% or higher. In some embodiments, the crystalline form has a purity of about 95% or higher. In some embodiments, the crystalline form has a purity of about 98% or higher. In some embodiments, the crystalline form comprises less than 0.5% of bromide ion (Br⁻) by weight. In some embodiments, the crystalline form is purified and isolated.

The present disclosure further provides a pharmaceutical composition comprising any one of the crystalline forms of the pamoate salt or solvate thereof and a pharmaceutically acceptable excipient or carrier. In some embodiments, the pharmaceutical composition comprises the crystalline form of the pamoate salt comprising less than 0.5% of bromide ion (Br⁻) by weight of the crystalline form of the pamoate salt.

In some embodiments, the pharmaceutical composition further comprising one or more additional therapeutically active agent. In some embodiments, the one or more additional therapeutically active agent is a cytotoxic agent or an anticancer agent. In some embodiments, the one or more additional therapeutically active agent is rituximab, docetaxel, carboplatin, paclitaxel, or prednisone.

In some embodiments, the pharmaceutical composition is formulated to be administered orally, nasally, transdermally, topically, pulmonary, inhalationally, buccally, sublingually, intraperintoneally, subcutaneously, intramuscularly, intravenously, rectally, intrapleurally, intrathecally, intraportally, or parenterally, or as an implant.

The present disclosure further provides a method of treating cancer comprising administering to a subject in need thereof, an effective amount of any one of the pamoate salt or solvate thereof as disclosed herein, any one of the crystalline form of the pamoate salt or solvate thereof as disclosed herein, or any one of the pharmaceutical composition comprising the pamoate salt or solvate thereof as disclosed herein.

In some embodiments of the method as disclosed herein, the cancer has a c-Myc rearrangement, amplification, or overexpression, and/or MYCN amplification or overexpression. In some embodiments, the cancer is a solid tumor or a hematologic malignancy. In some embodiments, the hematologic malignancy is myeloid disorder, lymphoid disorder, leukemia, lymphoma, myelodysplastic syndrome (MDS), myeloproliferative disease (MPD), mast cell disorder, or myeloma. In some embodiments, the hematologic malignancy is acute lymphoblastic leukemia (ALL), T-cell ALL (T-ALL), B-cell ALL (B-ALL), acute myeloid leukemia (AML), granulocytic leukemia, monocytic leukemia, lymphocytic leukemia, chronic lymphocytic leukemia (CLL), chronic myelogenous leukemia (CML), blast phase CML, small lymphocytic lymphoma (SLL), CLL/SLL, Hodgkin lymphoma (HL), non-Hodgkin lymphoma (NHL), B-cell NHL, CD20-positive B cell NHL, T-cell NHL, indolent NHL (iNHL), high-grade B-cell lymphoma (HGBCL), diffuse large B-cell lymphoma (DLBCL), mantle cell lymphoma (MCL), aggressive B-cell NHL, B-cell lymphoma (BCL), Richter's syndrome (RS), T-cell lymphoma (TCL), peripheral T-cell lymphoma (PTCL), cutaneous T-cell lymphoma (CTCL), transformed mycosis fungoides, Sezary syndrome, anaplastic large-cell lymphoma (ALCL), follicular lymphoma (FL), Waldenstrom macroglobulinemia (WM), lymphoplasmacytic lymphoma, Burkitt lymphoma, malignant lymphoma, plasmocytoma, reticulum cell sarcoma, multiple myeloma (MM), metastatic myeloma, amyloidosis, MPD, essential thrombocytosis (ET), myelofibrosis (MF), polycythemia vera (PV), chronic myelomonocytic leukemia (CMML), myelodysplastic syndrome (MDS), high-risk MDS, or low-risk MDS. In some embodiments, the hematologic malignancy is a high-grade B-cell lymphoma (HGBCL) or a diffuse large B-cell lymphoma (DLBCL). In some embodiments, the cancer is prostate cancer, breast cancer, lung cancer, brain cancer, liver cancer, uterine cancer, pancreatic cancer, gallbladder cancer, bile duct cancer, colon or rectum cancer, urinary bladder cancer, kidney cancer, ovarian cancer, esophageal cancer, gastric (stomach) cancer, head and neck cancer, bone cancer, thyroid cancer, cervical cancer, lymphoma, or leukemia. In some embodiments, the cancer is small cell lung cancer, non-small cell lung cancer (NSCLC), neuroendocrine prostate cancer (NEPC), hormone refractory prostate cancer (HRPC), neuroblastoma, triple negative breast cancer (TNBC), HER2 negative metastatic breast cancer, or hemangiosarcoma. In some embodiments, the cancer is relapsed or refractory.

In some embodiments of the method as disclosed herein, the method further comprising administering one or more additional therapeutically active agent. In some embodiments, the one or more additional therapeutically active agent is a cytotoxic agent or an anticancer agent. In some embodiments, the one or more additional therapeutically active agent is rituximab, docetaxel, carboplatin, paclitaxel, or prednisone.

The present disclosure further provides a method of inhibiting or reducing the expression of c-Myc protein level in a cancer cell of a subject, comprising administering to the subject, any one of the pamoate salt or solvate thereof as disclosed herein, any one of the crystalline form of the pamoate salt or solvate thereof as disclosed herein, or any one of the pharmaceutical composition comprising the pamoate salt or solvate thereof as disclosed herein.

The present disclosure further provides a method of degrading c-Myc protein in a cancer cell of a subject, comprising administering to the subject, any one of the pamoate salt or solvate thereof as disclosed herein, any one of the crystalline form of the pamoate salt or solvate thereof as disclosed herein, or any one of the pharmaceutical composition comprising the pamoate salt or solvate thereof as disclosed herein.

In some embodiments of any one of the methods as disclosed herein, the pamoate salt or solvate thereof, the crystalline form, or the pharmaceutical composition is administered to the subject orally, nasally, transdermally, topically, pulmonary, inhalationally, buccally, sublingually, intraperintoneally, subcutaneously, intramuscularly, intravenously, rectally, intrapleurally, intrathecally, intraportally, or parenterally, or as an implant.

### BRIEF DESCRIPTION OF THE DRAWINGS

FIG. 1A shows X-ray powder diffraction (XRPD) spectrum of crystalline Form A of the pamoate salt. FIG. 1B shows differential scanning calorimetry (DSC) thermograms of crystalline Form A of the pamoate salt. FIG. 1C shows thermogravimetric analysis (TGA) thermograms of crystalline Form A of the pamoate salt.

FIG. 2A shows XRPD spectrum of crystalline Form B of the pamoate salt. FIG. 2B shows DSC thermograms of crystalline Form B of the pamoate salt. FIG. 2C shows TGA thermograms of crystalline Form B of the pamoate salt.

FIG. 3A shows XRPD spectrum of crystalline Form C of the pamoate salt. FIG. 3B shows DSC thermograms of crystalline Form C of the pamoate salt. FIG. 3C shows TGA thermograms of crystalline Form C of the pamoate salt.

FIG. 4A shows XRPD spectrum of crystalline Form D of the pamoate salt. FIG. 4B shows DSC thermograms of crystalline Form D of the pamoate salt. FIG. 4C shows TGA thermograms of crystalline Form D of the pamoate salt.

FIG. 5 shows XRPD spectrum of crystalline Form E of the pamoate salt.

FIG. 6 shows XRPD spectrum of crystalline Form F of the pamoate salt.

FIG. 7 shows XRPD spectrum of crystalline Form G of the pamoate salt.

FIG. 8A shows XRPD spectrum of crystalline Form H of the pamoate salt. FIG. 8B shows DSC thermograms of crystalline Form H of the pamoate salt. FIG. 8C shows TGA thermograms of crystalline Form H of the pamoate salt.

FIG. 9A shows XRPD spectrum of crystalline Form I of the pamoate salt. FIG. 9B shows DSC thermograms of crystalline Form I of the pamoate salt. FIG. 9C shows TGA thermograms of crystalline Form I of the pamoate salt.

FIG. 10A shows XRPD spectrum of crystalline Form J of the pamoate salt. FIG. 10B shows DSC thermograms of crystalline Form J of the pamoate salt. FIG. 10C shows TGA thermograms of crystalline Form J of the pamoate salt.

FIG. 11 shows XRPD spectrum of two samples of amorphous form of the pamoate salt.

FIG. 12 shows c-Myc and Caspase-3 protein expression in SU-DHL-10 tumor in vivo after treatment with the pamoate salt.

FIG. 13A shows a time-course changes of tumor volume in SU-DHL-10 xenografted mice administrated with 1-(2-methoxyethyl)-2-methyl-4,9-dioxo-3-(pyrazin-2-ylmethyl)-4,9-dihydro-1H-naphtho[2,3-d]imidazolium bromide or the pamoate salt. FIG. 13B shows a time-course changes of body weight in SU-DHL-10 xenografted mice administrated with 1-(2-methoxyethyl)-2-methyl-4,9-dioxo-3-(pyrazin-2-ylmethyl)-4,9-dihydro-1H-naphtho[2,3-d]imidazolium bromide or the pamoate salt.

### DETAILED DESCRIPTION

All publications, patents and patent applications, including any drawings and appendices therein are incorporated by reference in their entirety for all purposes to the same extent as if each individual publication, patent or patent application, drawing, or appendix was specifically and individually indicated to be incorporated by reference in its entirety for all purposes.

### Definitions

While the following terms are believed to be well understood by one of ordinary skill in the art, the following definitions are set forth to facilitate explanation of the presently disclosed subject matter.

Throughout the present specification, the terms “about” and/or “approximately” may be used in conjunction with numerical values and/or ranges. The term “about” is understood to mean those values near to a recited value. Furthermore, the phrases “less than about [a value]” or “greater than about [a value]” should be understood in view of the definition of the term “about” provided herein. The terms “about” and “approximately” may be used interchangeably.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

The term “a” or “an” refers to one or more of that entity; for example, “an apoptosis inhibitor” refers to one or more apoptosis inhibitors or at least one apoptosis inhibitor. As such, the terms “a” (or “an”), “one or more” and “at least one” are used interchangeably herein. In addition, reference to “an inhibitor” by the indefinite article “a” or “an” does not exclude the possibility that more than one of the inhibitors is present, unless the context clearly requires that there is one and only one of the inhibitors.

As used herein, the verb “comprise” as is used in this description and in the claims and its conjugations are used in its non-limiting sense to mean that items following the word are included, but items not specifically mentioned are not excluded. The present invention may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The term “pharmaceutically acceptable salts” includes both acid and base addition salts. Pharmaceutically acceptable salts include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods.

As used herein, “solvate” means a complex formed by solvation (the combination of solvent molecules with molecules or ions of the active agent of the present invention), or an aggregate that consists of a solute ion or molecule (the active agent of the present invention) with one or more solvent molecules. In the present invention, the preferred solvate is hydrate. Examples of hydrate include, but are not limited to, hemihydrate, monohydrate, dihydrate, trihydrate, hexahydrate, etc. It should be understood by one of ordinary skill in the art that the pharmaceutically acceptable salt of the present compound may also exist in a solvate form (solvate salt). The solvate is typically formed via hydration which is either part of the preparation of the present compound or through natural absorption of moisture by the anhydrous compound of the present invention. Solvates including hydrates may be consisting in stoichiometric ratios, for example, with two, three, four salt molecules per solvate or per hydrate molecule. Another possibility, for example, that two salt molecules are stoichiometric related to three, five, seven solvent or hydrate molecules. Solvents used for crystallization, such as alcohols, especially methanol and ethanol; aldehydes; ketones, especially acetone; esters, e.g. ethyl acetate; may be embedded in the crystal grating. Preferred are pharmaceutically acceptable solvents.

The term “treating” means one or more of relieving, alleviating, delaying, reducing, improving, or managing at least one symptom of a condition in a subject. The term “treating” may also mean one or more of arresting, delaying the onset (i.e., the period prior to clinical manifestation of the condition) or reducing the risk of developing or worsening a condition.

An “effective amount” means the amount of a formulation according to the invention that, when administered to a patient for treating a state, disorder or condition is sufficient to effect such treatment. The “effective amount” will vary depending on the active ingredient, the state, disorder, or condition to be treated and its severity, and the age, weight, physical condition and responsiveness of the mammal to be treated.

The term “therapeutically effective” applied to dose or amount refers to that quantity of a compound or pharmaceutical formulation that is sufficient to result in a desired clinical benefit after administration to a patient in need thereof.

As used herein, a “subject” can be a human, non-human primate, mammal, rat, mouse, cow, horse, pig, sheep, goat, dog, cat and the like. The subject can be suspected of having or at risk for having a cancer, such as prostate cancer, breast cancer, ovarian cancer, salivary gland carcinoma, or endometrial cancer, or suspected of having or at risk for having acne, hirsutism, alopecia, benign prostatic hyperplasia, ovarian cysts, polycystic ovary disease, precocious puberty, spinal and bulbar muscular atrophy, or age-related macular degeneration. Diagnostic methods for various cancers, including but not limited to prostate cancer, breast cancer, ovarian cancer, lymphoma, and leukemia, are known to those of ordinary skill in the art.

“Mammal” includes humans and both domestic animals such as laboratory animals (e.g., mice, rats, monkeys, dogs, etc.) and household pets (e.g., cats, dogs, swine, cattle, sheep, goats, horses, rabbits), and non-domestic animals such as wildlife and the like.

All weight percentages (i.e., “% by weight” and “wt. %” and w/w) referenced herein, unless otherwise indicated, are measured relative to the total weight of the pharmaceutical composition.

As used herein, “substantially” or “substantial” refers to the complete or nearly complete extent or degree of an action, characteristic, property, state, structure, item, or result. For example, an object that is “substantially” enclosed would mean that the object is either completely enclosed or nearly completely enclosed. In another example, an XRPD pattern that is “substantially” similar to another XRPD pattern would mean that the one skilled in the art would understand the two patterns to be of the same substance in the same form. The exact allowable degree of deviation from absolute completeness may in some cases depend on the specific context. However, generally speaking, the nearness of completion will be so as to have the same overall result as if absolute and total completion were obtained. The use of “substantially” is equally applicable when used in a negative connotation to refer to the complete or near complete lack of action, characteristic, property, state, structure, item, or result. For example, a composition that is “substantially free of” other active agents would either completely lack other active agents, or so nearly completely lack other active agents that the effect would be the same as if it completely lacked other active agents. In other words, a composition that is “substantially free of” an ingredient or element or another active agent may still contain such an item as long as there is no measurable effect thereof.

Polymorphism can be characterized as the ability of a compound to crystallize into different crystal forms, while maintaining the same chemical formula. A crystalline polymorph of a given drug substance is chemically identical to any other crystalline polymorph of that drug substance in containing the same atoms bonded to one another in the same way, but differs in its crystal forms, which can affect one or more physical properties, such as stability, solubility, melting point, bulk density, flow properties, bioavailability, etc.

The following description includes information that may be useful in understanding the present invention. It is not an admission that any of the information provided herein is prior art or relevant to the presently claimed inventions, or that any publication specifically or implicitly referenced is prior art.

Sepantronium bromide (1-(2-methoxyethyl)-2-methyl-4,9-dioxo-3-(pyrazin-2-ylmethyl)-4,9-dihydro-1H-naphtho[2,3-d]imidazolium bromide), also known as YM155 monobromide, or YM155, is a small imidazolium-based proapoptotic agent, which is being developed for treating solid tumors and hematologic malignancies, including B-cell lymphomas and prostate cancer. Currently, sepantroinum bromide is being studied for a continuous IV infusion administration. See e.g., clinical trials with the corresponding identifiers: NCT01023386, NCT00818480, NCT00498914. Continuous infusion over multiple days provides many challenges, as it is not a desirable mode of administration for the patient. There is thus a need to provide sepantroium bromide or analog/derivative that could provide similar or better therapeutic efficacy, but has a much more desirable mode of administration for the patient. Such advantages could be for example a sepantroium bromide product or derivative product that can deliver longer release of the drug, thereby foregoing continuous or long infusion times, or that simply avoids IV infusion administration altogether.

### Pamoate Salt

The present disclosure relates to 1-(2-methoxyethyl)-2-methyl-4,9-dioxo-3-(pyrazin-2-ylmethyl)-4,9-dihydro-1H-naphtho[2,3-d]imidazolium pamoate (the “pamoate salt”) or solvate thereof. In one embodiment, the pamoate salt has the following structure, wherein n is in the range of about 0.8 to about 4.0. In one embodiment, n is about 1.0. In some embodiments, n is about 3.5. The pamoate salt has an aqueous solubility of less than 0.02 mg/mL at pH of about 7.4.

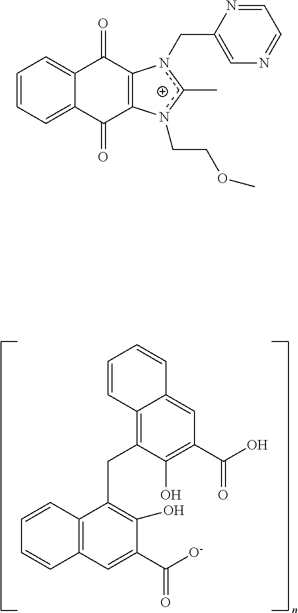

In one embodiment, the pamoate salt of the present disclosure is photostable. In one embodiment, the pamoate salt of the present disclosure remains unchanged or almost unchanged following 26.2 hours of irradiation under ICH Option 1 photostability test conditions (ID65 light, 250 W/m², 25° C./60% RH). In one embodiment, the pamoate salt of the present disclosure maintains its HPLC purity following 26.2 hours of irradiation under ICH Option 1 photostability test conditions.

In another embodiment, the pamoate salt or solvate thereof has a purity of at least about 99.9%, about 99.8%, about 99.7%, about 99.6%, about 99.5%, about 99.4%, about 99.3%, about 99.2%, about 99.1%, about 99.0%, about 98%, about 97%, about 96%, about 95%, about 94%, about 93%, about 92%, about 91%, or about 90%. In one embodiment, the pamoate salt or solvate thereof has a purity of at least about 99.9%, about 99.8%, about 99.7%, about 99.6%, about 99.5%, about 99.4%, about 99.3%, about 99.2%, about 99.1%, about 99.0%, about 98%, about 97%, about 96%, about 95%, about 94%, about 93%, about 92%, about 91%, or about 90% with minimal photodegradation. In a specific embodiment, the pamoate salt has a purity of at least about 99.9%, about 99.8%, about 99.7%, about 99.6%, about 99.5%, about 99.4%, about 99.3%, about 99.2%, about 99.1%, about 99.0%, about 98%, about 97%, about 96%, about 95%, about 94%, about 93%, about 92%, about 91%, or about 90% after light exposure/irradiation. In a specific embodiment, the light exposure/irradiation is as described in Example 16 herein. In another specific embodiment, the purity after light exposure is determined by XRPD, HPLC and/or appearance. In a specific embodiment, the pamoate salt is in the solid form. In another embodiment, the solid form is Form A. In another embodiment, the solid form is Form B.

In one embodiment, the present disclosure relates to an anhydrous or non-solvated form of the pamoate salt. In one embodiment, the present disclosure relates to a solvate of the pamoate salt. In one embodiment, the pamoate salt or solvate thereof is in a solid form. In one embodiment, the pamoate salt or solvate thereof is in a crystalline form. In one embodiment, the pamoate salt or solvate thereof is in an amorphous form.

In one embodiment, the solid form of the pamoate salt or solvate is in Form A, Form B, Form C, Form D, Form E, Form F, Form G, Form H, Form I, or Form J (collectively Forms A-J). In one embodiment, pamoate salt or solve of Forms A-J are crystalline. In one embodiment, the pamoate salt is pamoate salt From B.

In one embodiment, the present disclosure relates to an isolated solid form of the pamoate salt or solvate thereof. In one embodiment, the isolated solid form is an isolated crystalline form of the pamoate salt or solvate thereof. In one embodiment, the isolated solid form is in any one of crystalline Forms A-J. In one embodiment, the isolated pamoate salt is pamoate salt From B.

In one embodiment, the solid form of the pamoate or solvate thereof has a purity of at least about 75%, at least about 80%, at least about 85%, at least about 90%, at least about 95%, at least about 97%, at least about 98%, or at least about 99%. In one embodiment, the solid form of pamoate salt or solvate thereof has a purity of at least about 75%, at least about 80%, at least about 85%, at least about 90%, at least about 95%, at least about 97%, at least about 98%, or at least about 99% with respect to one specific solid form of the pamoate salt or solvate thereof.

In one embodiment, the solid form of the pamoate salt or solvate thereof has a purity of at least about 99.9%, about 99.8%, about 99.7%, about 99.6%, about 99.5%, about 99.4%, about 99.3%, about 99.2%, about 99.1%, about 99.0%, about 98%, about 97%, about 96%, about 95%, about 94%, about 93%, about 92%, about 91%, or about 90%. In one embodiment, the solid form of the pamoate salt or solvate thereof has a purity of at least about 99.9%, about 99.8%, about 99.7%, about 99.6%, about 99.5%, about 99.4%, about 99.3%, about 99.2%, about 99.1%, about 99.0%, about 98%, about 97%, about 96%, about 95%, about 94%, about 93%, about 92%, about 91%, or about 90% with respect to one specific solid form of the pamoate salt or solvate thereof.

In one embodiment, the solid form of the pamoate salt or solvate thereof has a purity of about 75% to about 99%. In one embodiment, the solid form of the pamoate salt or solvate thereof has a purity of about 80% to about 99%. In one embodiment, the solid form of the pamoate salt or solvate thereof has a purity of about 85% to about 99%. In one embodiment the solid form of the pamoate salt or solvate thereof has a purity of about 90% to about 99%. In one embodiment, the solid form of the pamoate salt or solvate thereof has a purity of about 95% to about 99%.

In one embodiment, the solid form of the pamoate salt or solvate thereof has a purity of about 75% to about 99% with respect to one specific solid form of the pamoate salt or solvate thereof. In one embodiment, the solid form of the pamoate salt or solvate thereof has a purity of about 80% to about 99% with respect to one specific solid form of the pamoate salt or solvate thereof. In one embodiment, the solid form of the pamoate salt or solvate thereof has a purity of about 85% to about 99% with respect to one specific solid form of the pamoate salt or solvate thereof. In one embodiment, the solid form of the pamoate salt or solvate thereof has a purity of about 90% to about 99% with respect to one specific solid form of the pamoate salt or solvate thereof. In one embodiment the solid form of the pamoate salt or solvate thereof has a purity of about 95% to about 99% with respect to one specific solid form of the pamoate salt or solvate thereof.

In one embodiment, the solid form of the pamoate salt or solvate thereof comprises less than about 0.5% of bromide ion (Br⁻) by weight of the pamoate salt or solvate thereof. In one embodiment, the solid form of the pamoate salt or solvate thereof comprises less than about 0.2% of bromide ion (Br⁻) by weight of the pamoate salt or solvate thereof. In one embodiment, the solid form of the pamoate salt or solvate thereof comprises less than about 1%, less than about 0.9%, less than about 0.8%, less than about 0.7%, less than about 0.6%, less than about 0.5%, less than about 0.4%, less than about 0.3%, or less than about 0.2% of bromide ion (Br⁻) by weight of the pamoate salt or solvate thereof.

In one embodiment, the specific solid form of the pamoate salt or solvate thereof with high purity is crystalline Form B. In one embodiment, Form B of the pamoate salt is the most thermodynamically stable form of the pamoate salt or solvate thereof among Forms A-J.

In one embodiment, the present disclosure relates to mixtures of solid forms of the pamoate salt or solvate thereof, wherein the mixture comprises one or more solid forms of the pamoate salt or solvate thereof.

### Crystalline Form of the Pamoate Salt or Solvate Thereof

In one embodiment, the present disclosure relates to a crystalline form of the pamoate salt or solvate thereof. In one embodiment, the present disclosure relates to an anhydrous or non-solvated crystalline form of the pamoate salt. In one embodiment, the present disclosure relates to solvate of the pamoate salt. In one embodiment, the present disclosure relates to a crystalline Forms A-J of the pamoate salt or solvate thereof.

In one embodiment, the crystalline form of the pamoate salt or solvate thereof comprises less than about 0.5% of bromide ion (Br⁻) by weight of the pamoate salt or solvate thereof. In one embodiment, the crystalline form of the pamoate salt or solvate thereof comprises less than about 0.2% of bromide ion (Br⁻) by weight of the pamoate salt or solvate thereof. In one embodiment, the crystalline form of the pamoate salt or solvate thereof comprises less than about 1%, less than about 0.9%, less than about 0.8%, less than about 0.7%, less than about 0.6%, less than about 0.5%, less than about 0.4%, less than about 0.3%, or less than about 0.2% of bromide ion (Br⁻) by weight of the pamoate salt or solvate thereof.

In one embodiment, the crystalline forms are characterized by the interlattice plane intervals determined by an X-ray powder diffraction (XRPD) pattern. The spectrum of XRPD is typically represented by a diagram plotting the intensity of the peaks versus the location of the peaks, i.e., diffraction angle 20 (two-theta) in degrees. The intensities are often given in parenthesis with the following abbreviations: very strong=vst; strong=st; medium=m; weak=w; and very weak=vw. The characteristic peaks of a given XRPD can be selected according to the peak locations and their relative intensity to conveniently distinguish this crystalline structure from others. The % intensity of the peaks relative to the most intense peak may be represented as I/Io.

Those skilled in the art recognize that the measurements of the XRPD peak locations and/or intensity for a given crystalline form of the same compound will vary within a margin of error. The values of degree 20 allow appropriate error margins. Typically, the error margins are represented by “±”. For example, the degree 2θ of about 10.6±0.2″ denotes a range from about 10.4 to 10.8 degree 2θ. Depending on the sample preparation techniques, the calibration techniques applied to the instruments, human operational variation, etc., those skilled in the art recognize that the appropriate error of margins for a XRPD can be about ±0.7; ±0.6; ±0.5; ±0.4; ±0.3; ±0.2; ±0.1; ±0.05; or less.

Additional details of the methods and equipment used for the XRPD analysis are described in the Examples section.

In one embodiment, the crystalline forms are characterized by Differential Scanning Calorimetry (DSC). The DSC thermogram is typically expressed by a diagram plotting the normalized heat flow in units of Watts/gram (“W/g”) versus the measured sample temperature in degree Celsius. The DSC thermogram is usually evaluated for extrapolated onset and end (outset) temperatures, peak temperature, and heat of fusion. A peak characteristic value of a DSC thermogram is often used as the characteristic peak to distinguish this crystalline structure from others.

Those skilled in the art recognize that the measurements of the DSC thermogram for a given crystalline form of the same compound will vary within a margin of error. The values of a single peak characteristic value, expressed in degrees Celsius, allow appropriate error margins. Typically, the error margins are represented by “±”. For example, the single peak characteristic value of about “170.5±0.2” degrees Celsius denotes a range from about 170.3 to 170.7 degrees Celsius. Depending on the sample preparation techniques, the calibration techniques applied to the instruments, human operational variations, etc., those skilled in the art recognize that the appropriate error of margins for a single peak characteristic value can be ±2.5; ±2.0; ±1.5; ±1.0; ±0.5; or less.

Additional details of the methods and equipment used for the DSC thermogram analysis are described in the Examples section.

In one embodiment, the crystalline forms are characterized by Dynamic Vapor Sorption (DVS). The DVS profile is typically expressed by a diagram plotting the sample relative humidity (RH) versus the change in mass (%). The DVS profile provides information on hygroscopicity of the crystalline form at different RH conditions.

Additional details of the methods and equipment used for DVS are described in the Examples section.

### Form A

In one embodiment, the present disclosure relates to a crystalline Form A of the pamoate salt. In one embodiment, Form A of the pamoate salt is an anhydrate (anhydrous or non-solvated). In one embodiment, Form A is of high crystallinity. In one embodiment, Form A has an imidazolium:pamoic acid stoichiometry of about 1:1.

In one embodiment, Form A of the pamoate salt may comprise of a mixture of one or more forms of the pamoate salt or solvate thereof. In some embodiments, the crystalline form of the pamoate salt or solvate thereof may comprise of substantially pure form of Form A. In one embodiment, the crystalline form of the pamoate salt or solvate thereof may comprise of over about 99.9%, about 99.8%, about 99.7%, about 99.6%, about 99.5%, about 99.4%, about 99.3%, about 99.2%, about 99.1%, or about 99.0% of Form A. In another embodiment, the crystalline form of the pamoate salt or solvate thereof may comprise over about 99%, 98%, 97%, 96%, 95%, 94%, 93%, 92%, 91%, or 90% of Form A. In some embodiments, the crystalline form of the pamoate salt or solvate thereof may comprise over about 90%, 85%, 80%, 75%, 70%, 65%, 60%, 55%, 50%, 45%, or 40% of Form A.

In one embodiment, Form A of the pamoate salt is isolated and purified. In one embodiment, the purified Form A of the pamoate salt does not comprise any detectable residual solvent by ¹H NMR. In one embodiment, the purified Form A of the pamoate salt is at least 95%, at least 96%, at least 97%, at least 98%, at least 99%, or at least 99.5% pure.

In one embodiment, crystalline Form A of the pamoate salt exhibits an XRPD pattern comprising peaks at about 8.08, 9.69, and 10.67 degrees two-theta with the margin of error of about ±0.5; about ±0.4; about ±0.3; about ±0.2; about ±0.1; about ±0.05; or less. In another embodiment, the XRPD of the crystalline Form A of the pamoate salt further comprises peaks at about 11.41, 15.29, and 16.17 degrees two-theta with the margin of error of about ±0.5; about ±0.4; about ±0.3; about ±0.2; about ±0.1; about ±0.05; or less. In further embodiment, the crystalline Form A of the pamoate salt further comprises at least two peaks selected from about 16.53, 22.31, 22.94, or 24.65 degrees two-theta with the margin of error of about ±0.5; about ±0.4; about ±0.3; about ±0.2; about ±0.1; about ±0.05; or less.

In one embodiment, crystalline Form A of the pamoate salt exhibits an XRPD pattern comprising peaks at about 8.08±0.2, 9.69±0.2, and 10.67±0.2 degrees two-theta. In one embodiment, crystalline Form A of the pamoate salt exhibits an XRPD pattern comprising three of the ten most intense peaks at about 8.08±0.2, 9.69±0.2, and 10.67±0.2 degrees two-theta. In one embodiment, crystalline Form A of the pamoate salt exhibits an XRPD pattern comprising three of the six most intense peaks at about 8.08±0.2, 9.69±0.2, and 10.67±0.2 degrees two-theta. In one embodiment, crystalline Form A of the pamoate salt exhibits an XRPD pattern comprising three most intense peaks at about 8.08±0.2, 9.69±0.2, and 10.67±0.2 degrees two-theta.

In one embodiment, crystalline Form A of the pamoate salt exhibits an XRPD pattern comprising peaks at about 11.41±0.2, 15.29±0.2, and 16.17±0.2 degrees two-theta. In one embodiment, crystalline Form A of the pamoate salt exhibits an XRPD pattern comprising three of the ten most intense peaks at about 11.41±0.2, 15.29±0.2, and 16.17±0.2 degrees two-theta. In one embodiment, crystalline Form A of the pamoate salt exhibits an XRPD pattern comprising three of the six most intense peaks at about 11.41±0.2, 15.29±0.2, and 16.17±0.2 degrees two-theta.

In one embodiment, crystalline Form A of the pamoate salt exhibits an XRPD pattern comprising at least two peaks selected from about 8.08±0.2, 9.69±0.2, 10.67±0.2, 11.41±0.2, 15.29±0.2, or 16.17±0.2 degrees two-theta. In one embodiment, crystalline Form A of the pamoate salt exhibits an XRPD pattern comprising at least three peaks selected from about 8.08±0.2, 9.69±0.2, 10.67±0.2, 11.41±0.2, 15.29±0.2, or 16.17±0.2 degrees two-theta. In one embodiment, crystalline Form A of the pamoate salt exhibits an XRPD pattern comprising at least four peaks selected from about 8.08±0.2, 9.69±0.2, 10.67±0.2, 11.41±0.2, 15.29±0.2, or 16.17±0.2 degrees two-theta. In one embodiment, crystalline Form A of the pamoate salt exhibits an XRPD pattern comprising at least five peaks selected from about 8.08±0.2, 9.69±0.2, 10.67±0.2, 11.41±0.2, 15.29±0.2, or 16.17±0.2 degrees two-theta. In one embodiment, crystalline Form A of the pamoate salt exhibits an XRPD pattern comprising peaks at about 8.08±0.2, 9.69±0.2, 10.67±0.2, 11.41±0.2, 15.29±0.2, and 16.17±0.2 degrees two-theta.

In one embodiment, crystalline Form A of the pamoate salt exhibits an XRPD pattern comprising at least two peaks, at least three peaks, at least four peaks, at least five peaks, at least six peaks, at least seven peaks, at least eight peaks, or at least nine peaks selected from about 8.08±0.2, 9.69±0.2, 10.67±0.2, 11.41±0.2, 15.29±0.2, 16.17±0.2, 16.53±0.2, 22.31±0.2, 22.94±0.2, or 24.65±0.2 degrees two-theta. In one embodiment, crystalline Form A of the pamoate salt exhibits an XRPD pattern comprising peaks at about 8.08±0.2, 9.69±0.2, 10.67±0.2, 11.41±0.2, 15.29±0.2, 16.17±0.2, 16.53±0.2, 22.31±0.2, 22.94±0.2, and 24.65±0.2 degrees two-theta.

In one embodiment, the crystalline Form A of the pamoate salt exhibits an XRPD comprising peaks shown in Table 1 below. In one embodiment, the crystalline Form A of the pamoate salt exhibits an XRPD comprising at least five most intense peaks shown in Table 1 below (net intensity). In one embodiment, the crystalline Form A of the pamoate salt exhibits an XRPD comprising at least six, at least seven, at least eight, at least nine, at least ten most intense peaks shown in Table 1 below (net intensity).

TABLE 1
XRPD Table of Form A of the pamoate salt

| Angle | D Value | Net | Gross |
|---|---|---|---|
| (°2θ) | (Å) | Intensity | Intensity |
| 11.413 | 7.74689 | 986.632 | 1092.41 |
| 16.165 | 5.47871 | 1125.32 | 1234.55 |
| 16.964 | 5.22247 | 39.8227 | 144.031 |
| 11.935 | 7.40902 | 54.2459 | 158.657 |
| 8.081 | 10.93207 | 1503.52 | 1582.38 |
| 10.674 | 8.28125 | 1569.41 | 1676.21 |
| 12.327 | 7.17426 | 76.5373 | 180.946 |
| 24.336 | 3.65450 | 41.4304 | 182.827 |
| 36.567 | 2.45540 | 78.4237 | 192.134 |
| 13.026 | 6.79097 | 89.8986 | 195.327 |
| 33.386 | 2.68168 | 59.1789 | 196.167 |
| 19.422 | 4.56661 | 74.8929 | 201.952 |
| 28.439 | 3.13591 | 56.4165 | 215.795 |
| 25.535 | 3.48564 | 80.0162 | 216.588 |
| 20.360 | 4.35845 | 91.4858 | 224.928 |
| 31.969 | 2.79726 | 109.270 | 237.719 |
| 19.860 | 4.46692 | 113.256 | 243.054 |
| 23.983 | 3.70753 | 110.811 | 254.377 |
| 26.917 | 3.30970 | 98.4292 | 259.213 |
| 32.637 | 2.74150 | 137.940 | 270.437 |
| 21.789 | 4.07571 | 122.847 | 270.444 |
| 23.611 | 3.76501 | 133.749 | 277.888 |
| 9.685 | 9.12533 | 2676.12 | 2773.41 |
| 28.971 | 3.07950 | 128.496 | 280.443 |
| 20.488 | 4.33143 | 159.620 | 293.482 |
| 22.091 | 4.02065 | 146.099 | 294.957 |
| 29.507 | 3.02480 | 160.059 | 300.897 |
| 21.009 | 4.22514 | 212.701 | 351.702 |
| 18.064 | 4.90674 | 234.696 | 353.807 |
| 28.073 | 3.17594 | 237.064 | 399.466 |
| 21.420 | 4.14504 | 301.659 | 446.150 |
| 13.955 | 6.34099 | 373.296 | 473.762 |
| 17.620 | 5.02938 | 371.837 | 483.371 |
| 26.282 | 3.38823 | 332.367 | 485.040 |
| 13.248 | 6.67772 | 432.796 | 538.034 |
| 18.684 | 4.74544 | 446.447 | 571.951 |
| 16.533 | 5.35763 | 479.106 | 587.030 |
| 27.225 | 3.27294 | 444.229 | 607.107 |
| 22.940 | 3.87364 | 466.041 | 612.223 |
| 22.307 | 3.98218 | 742.032 | 891.076 |
| 24.647 | 3.60908 | 784.296 | 922.467 |
| 15.290 | 5.79015 | 873.117 | 978.551 |

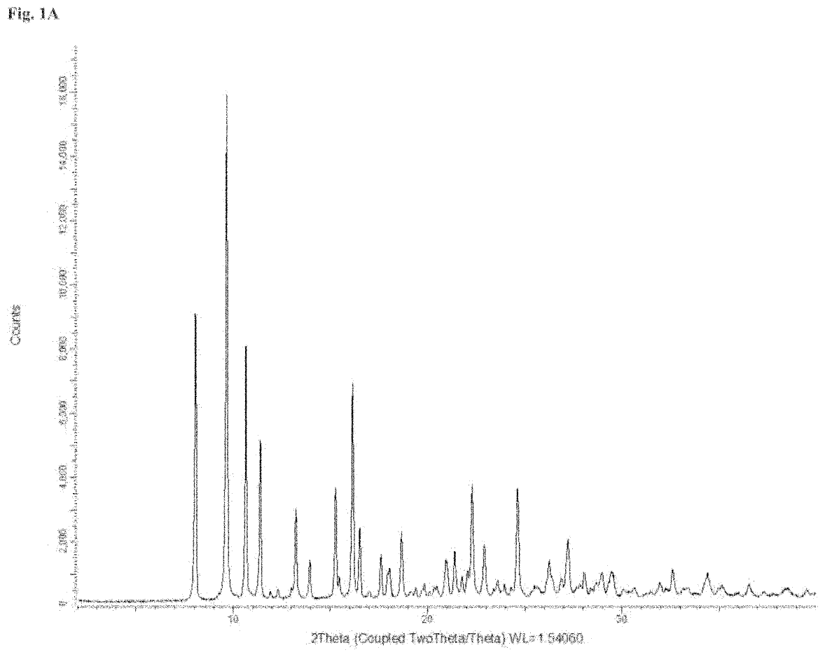

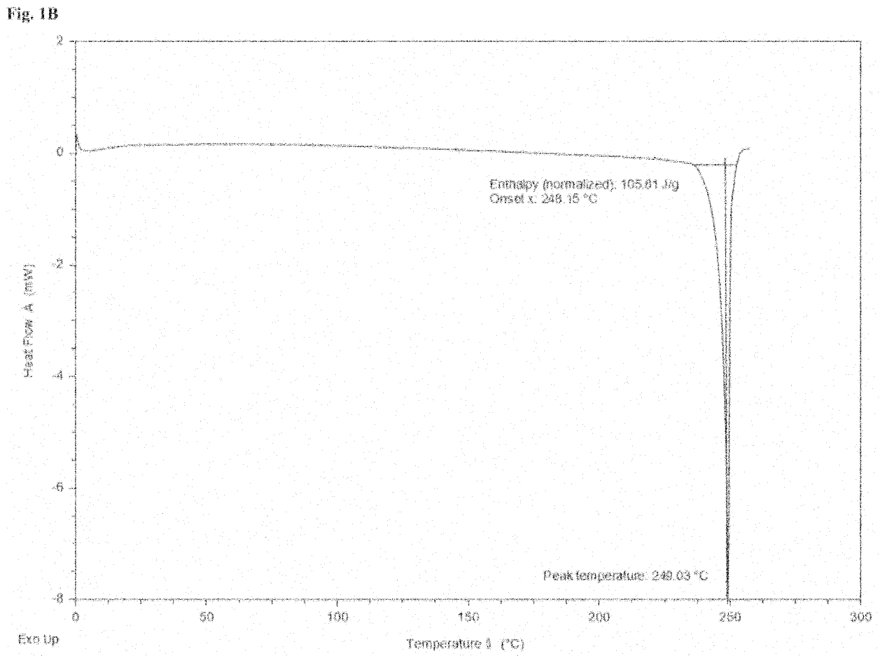

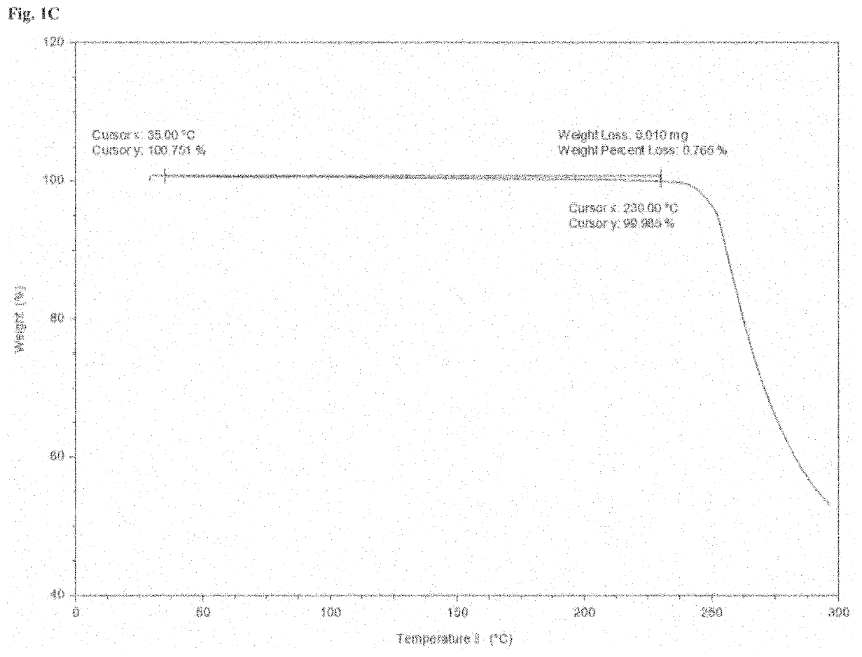

In one specific embodiment, the crystalline Form A of the pamoate salt exhibits an XRPD pattern that is substantially similar to FIG. 1A.

In one embodiment, the crystalline Form A of the pamoate salt exhibits a DSC thermogram comprising an endotherm peak at about 248° C. (onset) with the error of margin of about ±2.5; about ±2.0; about ±1.5; about ±1.0; about ±0.5; or less. In one embodiment, the crystalline Form A of the pamoate salt exhibits a DSC thermogram comprising an endotherm peak at about 249° C. (peak maximum) with the error of margin of about ±2.5; about ±2.0; about ±1.5; about 1.0; about ±0.5; or less. In one embodiment, the crystalline Form A of the pamoate salt exhibits a DSC thermogram that is substantially similar to FIG. 1B.

In one embodiment, the crystalline Form A of the pamoate salt exhibits a TGA thermogram substantially similar to FIG. 1C. In one embodiment, crystalline Form A of the pamoate salt shows about 0.8% weight loss in the range of about 35° C. to about 230° C. by TGA.

### Form B

In one embodiment, the present disclosure relates to a crystalline Form B of the pamoate salt. In one embodiment, Form B of the pamoate salt is an anhydrate (anhydrous or non-solvated). In one embodiment, Form B is of high crystallinity. In one embodiment, Form B has an imidazolium:pamoic acid stoichiometry of about 1:1.

In one embodiment, Form B of the pamoate salt may comprise of a mixture of one or more forms of the pamoate salt or solvate thereof. In some embodiments, the crystalline form of the pamoate salt or solvate thereof may comprise of substantially pure form of Form B. In one embodiment, the crystalline form of the pamoate salt or solvate thereof may comprise of over about 99.9%, about 99.8%, about 99.7%, about 99.6%, about 99.5%, about 99.4%, about 99.3%, about 99.2%, about 99.1%, or about 99.0% of Form B. In another embodiment, the crystalline form of the pamoate salt or solvate thereof may comprise over about 99%, 98%, 97%, 96%, 95%, 94%, 93%, 92%, 91%, or 90% of Form B. In some embodiments, the crystalline form of the pamoate salt or solvate thereof may comprise over about 90%, 85%, 80%, 75%, 70%, 65%, 60%, 55%, 50%, 45%, or 40% of Form B.

In one embodiment, Form B of the pamoate salt is isolated and purified. In one embodiment, the purified Form B of the pamoate salt does not comprise any detectable residual solvent by ¹H NMR. In one embodiment, the purified Form B of the pamoate salt is at least 95%, at least 96%, at least 97%, at least 98%, at least 99%, or at least 99.5% pure.

In one embodiment, crystalline Form B of the pamoate salt exhibits an XRPD pattern comprising peaks at about 9.32 and 16.89 degrees two-theta with the margin of error of about ±0.5; about ±0.4; about ±0.3; about ±0.2; about 0.1; about ±0.05; or less. In another embodiment, the XRPD of the crystalline Form B of the pamoate salt further comprises peaks at about 10.97, 11.60, and 24.77 degrees two-theta with the margin of error of about ±0.5; about ±0.4; about ±0.3; about ±0.2; about ±0.1; about ±0.05; or less. In further embodiment, the crystalline Form B of the pamoate salt further comprises at least two peaks selected from about 15.76, 18.04, 18.46, 21.16, or 22.04 degrees two-theta with the margin of error of about ±0.5; about ±0.4; about ±0.3; about ±0.2; about ±0.1; about ±0.05; or less.

In one embodiment, crystalline Form B of the pamoate salt exhibits an XRPD pattern comprising peaks at about 9.32±0.2 and 16.89±0.2 degrees two-theta. In one embodiment, crystalline Form B of the pamoate salt exhibits an XRPD pattern comprising two of the ten most intense peaks at about 9.32±0.2 and 16.89±0.2 degrees two-theta. In one embodiment, crystalline Form B of the pamoate salt exhibits an XRPD pattern comprising two of the five most intense peaks at about 9.32±0.2 and 16.89±0.2 degrees two-theta. In one embodiment, crystalline Form B of the pamoate salt exhibits an XRPD pattern comprising two of the three most intense peaks at about 9.32±0.2 and 16.89±0.2 degrees two-theta.

In one embodiment, crystalline Form B of the pamoate salt exhibits an XRPD pattern comprising peaks at about 10.97±0.2, 11.60±0.2, and 24.77±0.2 degrees two-theta. In one embodiment, crystalline Form B of the pamoate salt exhibits an XRPD pattern comprising three of the ten most intense peaks at about 10.97±0.2, 11.60±0.2, and 24.77±0.2 degrees two-theta. In one embodiment, crystalline Form B of the pamoate salt exhibits an XRPD pattern comprising three of the five most intense peaks at about 10.97±0.2, 11.60±0.2, and 24.77±0.2 degrees two-theta.

In one embodiment, crystalline Form B of the pamoate salt exhibits an XRPD pattern comprising at least two peaks selected from about 9.32±0.2, 10.97±0.2, 11.60±0.2, 16.89±0.2, or 24.77±0.2 degrees two-theta. In one embodiment, crystalline Form B of the pamoate salt exhibits an XRPD pattern comprising at least three peaks selected from about 9.32±0.2, 10.97±0.2, 11.60±0.2, 16.89±0.2, or 24.77±0.2 degrees two-theta. In one embodiment, crystalline Form B of the pamoate salt exhibits an XRPD pattern comprising at least four peaks selected from about 9.32±0.2, 10.97±0.2, 11.60±0.2, 16.89±0.2, or 24.77±0.2 degrees two-theta. In one embodiment, crystalline Form B of the pamoate salt exhibits an XRPD pattern comprising peaks at about 9.32±0.2, 10.97±0.2, 11.60±0.2, 16.89±0.2, and 24.77±0.2 degrees two-theta.

In one embodiment, crystalline Form B of the pamoate salt exhibits an XRPD pattern comprising at least two peaks, at least three peaks, at least four peaks, at least five peaks, at least six peaks, at least seven peaks, at least eight peaks, or at least nine peaks selected from about 9.32±0.2, 10.97±0.2, 11.60±0.2, 15.76±0.2, 16.89±0.2, 18.04±0.2, 18.46±0.2, 21.16±0.2, 22.04±0.2, or 24.77±0.2 degrees two-theta. In one embodiment, crystalline Form B of the pamoate salt exhibits an XRPD pattern comprising peaks at about 9.32±0.2, 10.97±0.2, 11.60±0.2, 15.76±0.2, 16.89±0.2, 18.04±0.2, 18.46±0.2, 21.16±0.2, 22.04±0.2, and 24.77±0.2 degrees two-theta.

In one embodiment, the crystalline Form B of the pamoate salt exhibits an XRPD comprising peaks shown in Table 2 below. In one embodiment, the crystalline Form B of the pamoate salt exhibits an XRPD comprising at least five most intense peaks shown in Table 2 below (net intensity). In one embodiment, the crystalline Form B of the pamoate salt exhibits an XRPD comprising at least six, at least seven, at least eight, at least nine, at least ten most intense peaks shown in Table 2 below (net intensity).

TABLE 2
XRPD Table of Form B of the pamoate salt

| Angle | D Value | Net | Gross |
|---|---|---|---|
| (°2θ) | (Å) | Intensity | Intensity |
| 6.680 | 13.22162 | 34.8890 | 94.6328 |
| 7.550 | 11.70040 | 291.852 | 356.169 |
| 9.322 | 9.47916 | 6415.62 | 6496.07 |
| 10.968 | 8.06039 | 619.841 | 711.547 |
| 11.598 | 7.62405 | 954.703 | 1050.35 |
| 12.062 | 7.33148 | 244.983 | 338.580 |
| 12.556 | 7.04395 | 221.563 | 312.530 |
| 13.296 | 6.65388 | 53.0151 | 139.763 |
| 14.518 | 6.09642 | 154.379 | 244.243 |
| 14.857 | 5.95786 | 240.258 | 335.886 |
| 15.070 | 5.87422 | 399.995 | 498.094 |
| 15.760 | 5.61842 | 419.716 | 519.757 |
| 16.889 | 5.24556 | 1862.92 | 1960.45 |
| 18.035 | 4.91476 | 418.641 | 523.141 |
| 18.461 | 4.80221 | 441.068 | 544.494 |
| 18.922 | 4.68617 | 104.562 | 202.835 |
| 19.700 | 4.50283 | 121.135 | 215.225 |
| 20.044 | 4.42625 | 113.112 | 208.292 |
| 20.616 | 4.30486 | 281.660 | 383.902 |
| 21.161 | 4.19510 | 585.055 | 702.692 |
| 22.039 | 4.02990 | 439.427 | 569.653 |
| 22.354 | 3.97381 | 173.428 | 304.499 |
| 22.781 | 3.90038 | 367.093 | 496.240 |
| 22.983 | 3.86649 | 247.766 | 374.756 |
| 23.263 | 3.82068 | 239.057 | 361.768 |
| 24.281 | 3.66273 | 347.134 | 470.811 |
| 24.767 | 3.59191 | 769.345 | 903.911 |
| 25.060 | 3.55059 | 317.145 | 456.047 |
| 25.595 | 3.47762 | 350.563 | 493.287 |
| 25.902 | 3.43698 | 165.623 | 308.158 |
| 26.722 | 3.33346 | 181.399 | 324.691 |
| 26.961 | 3.30438 | 306.435 | 448.464 |
| 27.275 | 3.26704 | 265.759 | 404.437 |
| 28.531 | 3.12600 | 156.738 | 289.811 |
| 28.704 | 3.10757 | 235.236 | 367.682 |
| 29.195 | 3.05644 | 109.717 | 237.219 |
| 30.218 | 2.95519 | 256.110 | 385.183 |
| 30.557 | 2.92320 | 226.203 | 358.734 |
| 30.877 | 2.89365 | 190.610 | 324.347 |
| 31.520 | 2.83608 | 87.2659 | 217.396 |
| 34.656 | 2.58629 | 245.912 | 358.749 |
| 36.646 | 2.45027 | 122.401 | 241.350 |

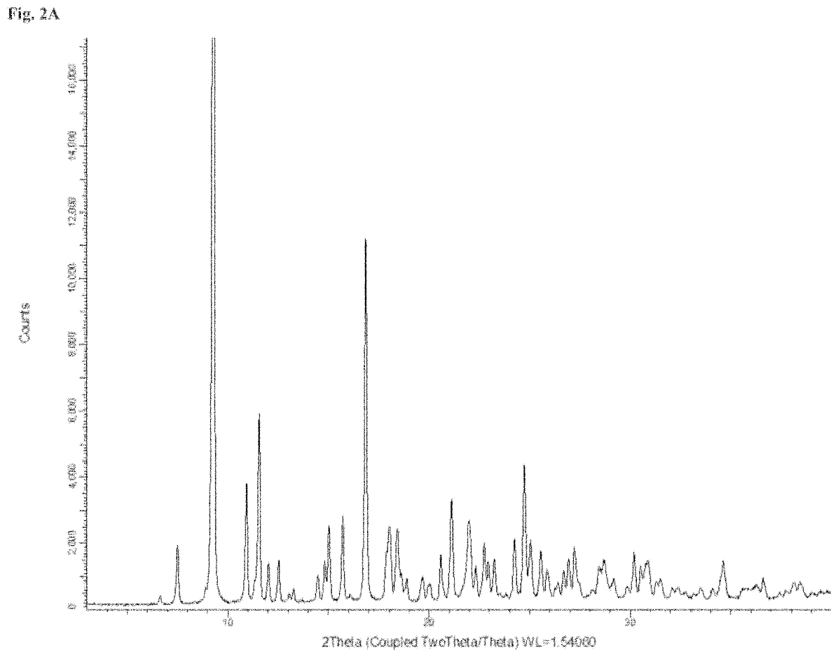

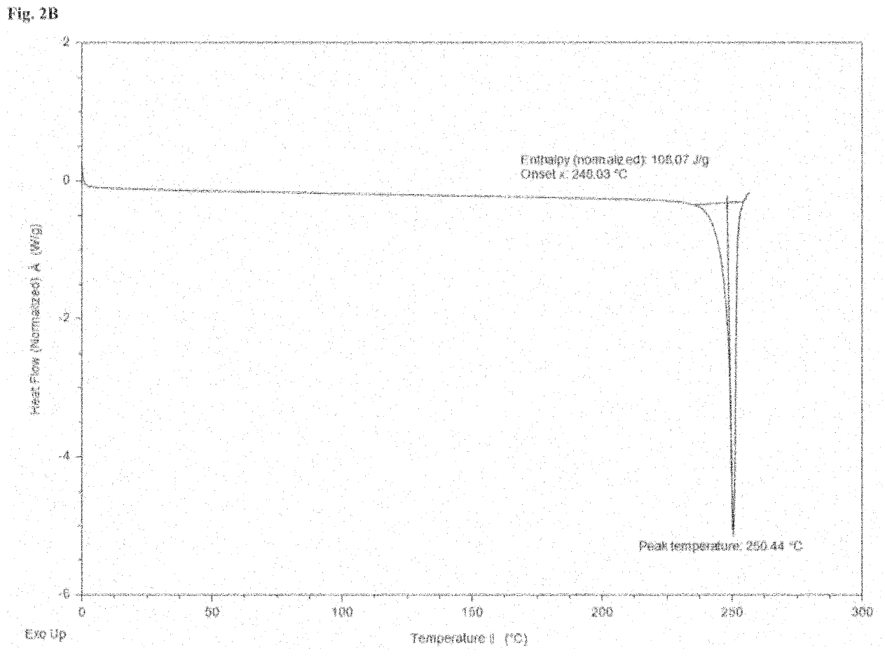

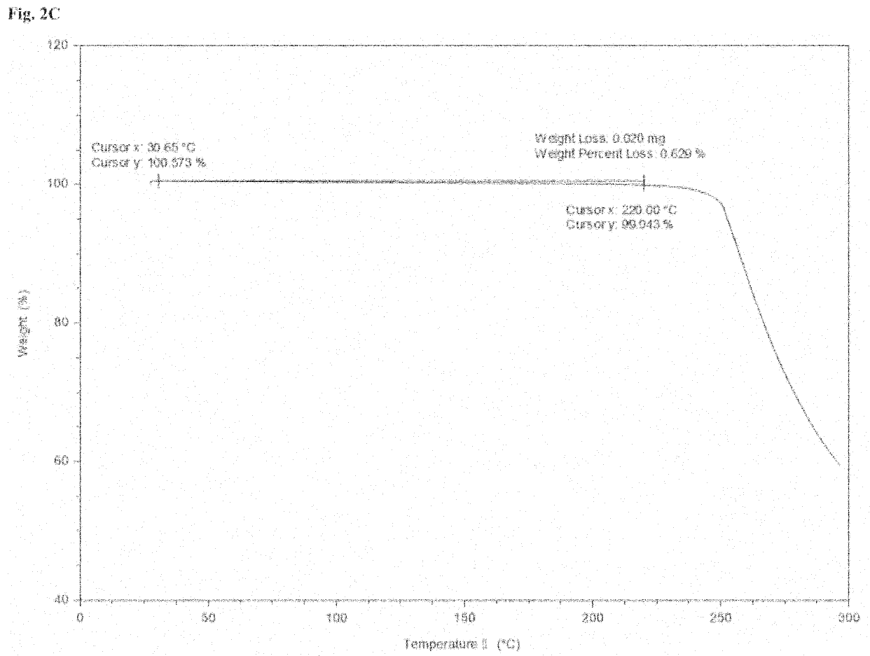

In one specific embodiment, the crystalline Form B of the pamoate salt exhibits an XRPD pattern that is substantially similar to FIG. 2A.

In one embodiment, the crystalline Form B of the pamoate salt exhibits a DSC thermogram comprising an endotherm peak at about 248° C. (onset) with the error of margin of about ±2.5; about ±2.0; about ±1.5; about ±1.0; about ±0.5; or less. In one embodiment, the crystalline Form B of the pamoate salt exhibits a DSC thermogram comprising an endotherm peak at about 250° C. (peak maximum) with the error of margin of about ±2.5; about ±2.0; about ±1.5; about ±1.0; about ±0.5; or less. In one embodiment, the crystalline Form B of the pamoate salt exhibits a DSC thermogram that is substantially similar to FIG. 2B.

In one embodiment, the crystalline Form B of the pamoate salt exhibits a TGA thermogram substantially similar to FIG. 2C. In one embodiment, crystalline Form B of the pamoate salt shows about 0.6% weight loss in the range of about 35° C. to about 220° C. by TGA.

### Form C

In one embodiment, the present disclosure relates to a crystalline Form C of the pamoate salt solvate. In one embodiment, Form C of the pamoate salt is an ethanol solvate. In one embodiment, Form C is of medium crystallinity. In one embodiment, Form C has an imidazolium:pamoic acid stoichiometry of about 1:1. In one embodiment, Form C comprises ethanol in about 0.3 equiv. as determined by ¹H NMR. In one embodiment, Form C comprises ethanol in about 1.8% by weight.

In one embodiment, Form C of the pamoate salt solvate may comprise of a mixture of one or more forms of the pamoate salt or solvate thereof. In some embodiments, the crystalline form of the pamoate salt or solvate thereof may comprise of substantially pure form of Form C. In one embodiment, the crystalline form of the pamoate salt or solvate thereof may comprise of over about 99.9%, about 99.8%, about 99.7%, about 99.6%, about 99.5%, about 99.4%, about 99.3%, about 99.2%, about 99.1%, or about 99.0% of Form C. In another embodiment, the crystalline form of the pamoate salt or solvate thereof may comprise over about 99%, 98%, 97%, 96%, 95%, 94%, 93%, 92%, 91%, or 90% of Form C. In some embodiments, the crystalline form of the pamoate salt or solvate thereof may comprise over about 90%, 85%, 80%, 75%, 70%, 65%, 60%, 55%, 50%, 45%, or 40% of Form C.

In one embodiment, Form C of the pamoate salt solvate comprises one or both of Form A and Form B of the pamoate salt.

In one embodiment, Form C of the pamoate salt solvate is isolated and purified. In one embodiment, the purified Form C of the pamoate salt solvate is at least 95%, at least 96%, at least 97%, at least 98%, at least 99%, or at least 99.5% pure.

In one embodiment, crystalline Form C of the pamoate salt solvate exhibits an XRPD pattern comprising peaks at about 7.39 and 26.03 degrees two-theta with the margin of error of about ±0.5; about ±0.4; about ±0.3; about ±0.2; about ±0.1; about ±0.05; or less. In one embodiment, crystalline Form C of the pamoate salt solvate exhibits an XRPD pattern comprising peaks at about 7.39, 10.12, and 26.03 degrees two-theta with the margin of error of about ±0.5; about ±0.4; about ±0.3; about ±0.2; about ±0.1; about ±0.05; or less. In another embodiment, the XRPD of the crystalline Form C further comprises peaks at about 10.12, 21.26, and 25.70 degrees two-theta with the margin of error of about ±0.5; about ±0.4; about ±0.3; about ±0.2; about ±0.1; about ±0.05; or less.

In one embodiment, crystalline Form C of the pamoate salt solvate exhibits an XRPD pattern comprising peaks at about 7.39±0.2 and 26.03±0.2 degrees two-theta. In one embodiment, crystalline Form C exhibits an XRPD pattern comprising two of the ten most intense peaks at about 7.39±0.2 and 26.03±0.2 degrees two-theta. In one embodiment, crystalline Form C exhibits an XRPD pattern comprising two of the five most intense peaks at about 7.39±0.2 and 26.03±0.2 degrees two-theta.

In one embodiment, crystalline Form C of the pamoate salt solvate exhibits an XRPD pattern comprising peaks at about 10.12±0.2, 21.26±0.2, and 25.70±0.2 degrees two-theta. In one embodiment, crystalline Form C exhibits an XRPD pattern comprising three of the ten most intense peaks at about 10.12±0.2, 21.26±0.2, and 25.70±0.2 degrees two-theta. In one embodiment, crystalline Form C exhibits an XRPD pattern comprising three of the five most intense peaks at about 10.12±0.2, 21.26±0.2, and 25.70±0.2 degrees two-theta.

In one embodiment, crystalline Form C of the pamoate salt solvate exhibits an XRPD pattern comprising at least two peaks selected from about 7.39±0.2, 10.12±0.2, 21.26±0.2, 25.70±0.2, or 26.03±0.2 degrees two-theta. In one embodiment, crystalline Form C of the pamoate salt solvate exhibits an XRPD pattern comprising at least three peaks selected from about 7.39±0.2, 10.12±0.2, 21.26±0.2, 25.70±0.2, or 26.03±0.2 degrees two-theta. In one embodiment, crystalline Form C of the pamoate salt solvate exhibits an XRPD pattern comprising at least four peaks selected from about 7.39±0.2, 10.12±0.2, 21.26±0.2, 25.70±0.2, or 26.03±0.2 degrees two-theta. In one embodiment, crystalline Form C of the pamoate salt solvate exhibits an XRPD pattern comprising peaks at about 7.39±0.2, 10.12±0.2, 21.26±0.2, 25.70±0.2, and 26.03±0.2 degrees two-theta.

In one embodiment, crystalline Form C of the pamoate salt solvate exhibits an XRPD pattern comprising at least two peaks, at least three peaks, at least four peaks, at least five peaks, at least six peaks, at least seven peaks, at least eight peaks, or at least nine peaks selected from about 7.39±0.2, 9.18±0.2, 10.12±0.2, 11.05±0.2, 21.26±0.2, 22.10±0.2, 22.73±0.2, 25.70±0.2, and 26.03±0.2, or 26.97±0.2 degrees two-theta. In one embodiment, crystalline Form C of the pamoate salt solvate exhibits an XRPD pattern comprising peaks at about 7.39±0.2, 9.18±0.2, 10.12±0.2, 11.05±0.2, 21.26±0.2, 22.10±0.2, 22.73±0.2, 25.70±0.2, and 26.03±0.2, and 26.97±0.2 degrees two-theta.

In one embodiment, the crystalline Form C of the pamoate salt solvate exhibits an XRPD comprising peaks shown in Table 3 below. In one embodiment, the crystalline Form C of the pamoate salt exhibits an XRPD comprising at least five most intense peaks shown in Table 3 below (net intensity). In one embodiment, the crystalline Form C of the pamoate salt exhibits an XRPD comprising at least six, at least seven, at least eight, at least nine, at least ten most intense peaks shown in Table 3 below (net intensity).

TABLE 3
XRPD Table of Form C of the pamoate salt solvate

| Angle (°2θ) | D Value (Å) | Net Intensity | Gross Intensity |
|---|---|---|---|
| 3.547 | 24.89142 | 16.0583 | 37.2359 |
| 4.575 | 19.30060 | 19.1962 | 41.3684 |
| 4.906 | 17.99711 | 12.6429 | 35.3133 |
| 7.393 | 11.94718 | 669.205 | 694.619 |
| 9.179 | 9.62717 | 196.454 | 230.891 |
| 9.687 | 9.12274 | 23.3133 | 61.0769 |
| 10.116 | 8.73747 | 421.765 | 462.328 |
| 11.051 | 7.99973 | 323.069 | 365.930 |
| 11.338 | 7.79804 | 23.5035 | 66.0190 |
| 12.257 | 7.21520 | 31.3882 | 74.4289 |
| 13.988 | 6.32593 | 52.1084 | 116.477 |
| 14.515 | 6.09742 | 162.419 | 234.640 |
| 14.958 | 5.91805 | 126.108 | 203.635 |
| 15.976 | 5.54320 | 121.888 | 213.000 |
| 16.631 | 5.32630 | 61.0997 | 165.483 |
| 16.894 | 5.24400 | 98.5980 | 209.372 |
| 17.205 | 5.14979 | 135.464 | 253.273 |
| 17.995 | 4.92556 | 43.8148 | 176.861 |
| 18.198 | 4.87110 | 41.1680 | 177.529 |
| 18.939 | 4.68213 | 46.5783 | 192.942 |
| 19.556 | 4.53564 | 103.239 | 255.430 |
| 20.102 | 4.41363 | 128.494 | 283.934 |
| 21.256 | 4.17652 | 460.515 | 616.949 |
| 21.689 | 4.09414 | 154.960 | 309.711 |
| 22.096 | 4.01966 | 204.409 | 356.556 |
| 22.730 | 3.90906 | 300.416 | 446.533 |
| 22.926 | 3.87609 | 161.590 | 305.357 |
| 23.403 | 3.79805 | 94.2945 | 231.369 |
| 25.243 | 3.52532 | 40.1378 | 159.034 |
| 25.697 | 3.46396 | 386.330 | 505.181 |
| 26.033 | 3.42005 | 766.851 | 884.876 |
| 26.515 | 3.35891 | 64.8938 | 180.549 |
| 26.966 | 3.30374 | 242.726 | 354.906 |
| 28.100 | 3.17294 | 34.2673 | 132.330 |
| 29.665 | 3.00903 | 39.3089 | 121.446 |
| 29.888 | 2.98706 | 27.4577 | 108.674 |
| 30.204 | 2.95655 | 97.4300 | 176.833 |
| 30.498 | 2.92872 | 25.2264 | 102.406 |
| 31.265 | 2.85857 | 116.859 | 185.797 |
| 31.860 | 2.80660 | 29.1957 | 92.8666 |
| 31.974 | 2.79680 | 86.8605 | 150.872 |
| 32.372 | 2.76332 | 83.0693 | 147.661 |
| 32.912 | 2.71924 | 24.6703 | 88.5345 |
| 33.400 | 2.68058 | 65.3303 | 127.033 |
| 34.431 | 2.60262 | 27.2712 | 84.3868 |
| 36.542 | 2.45699 | 30.3473 | 86.7368 |

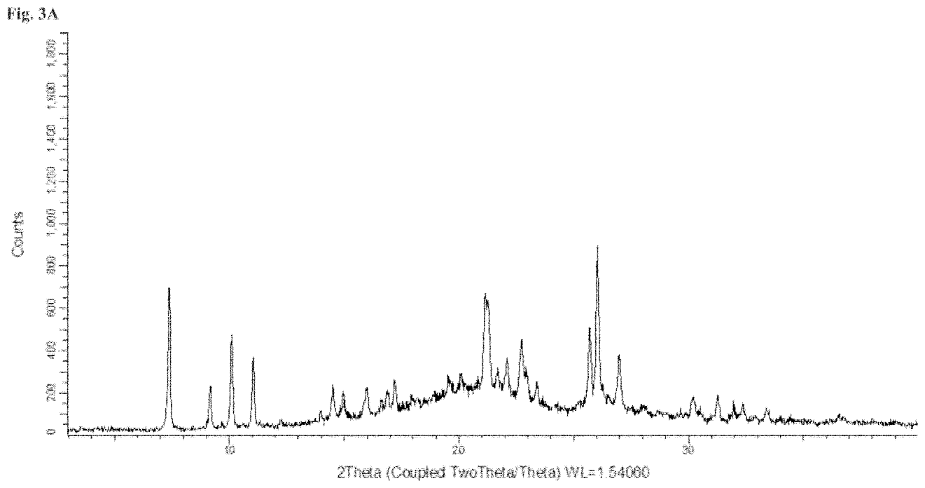

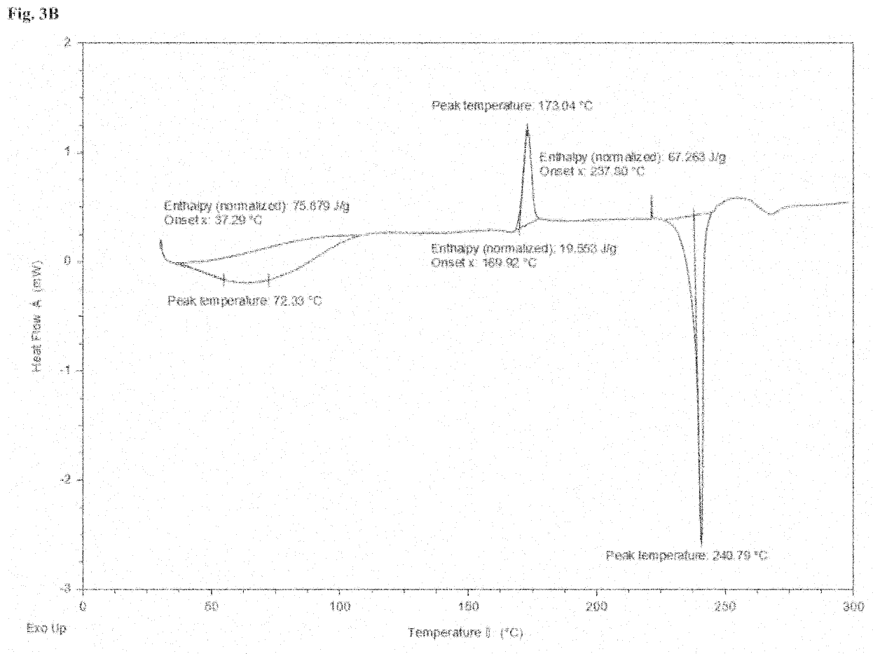

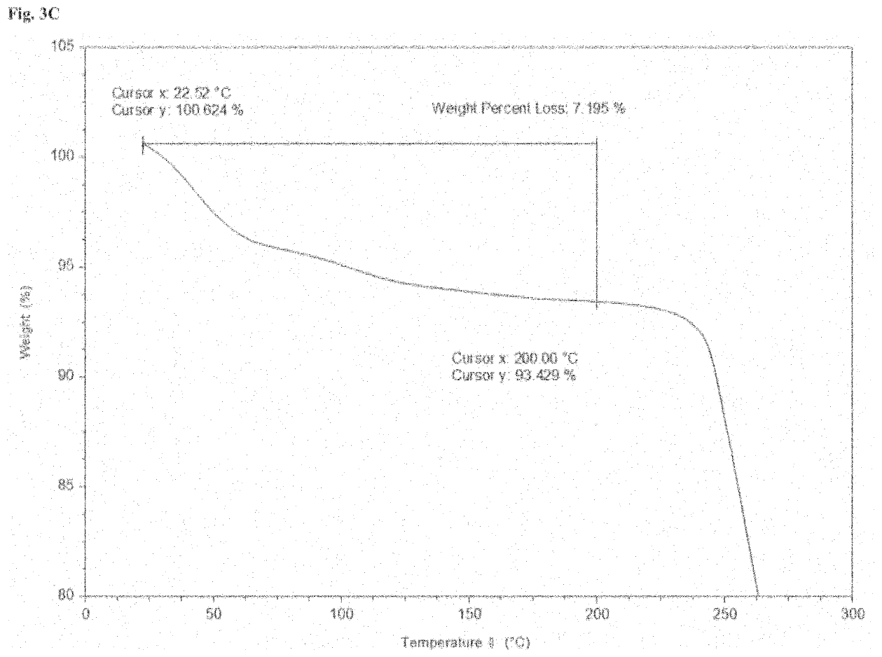

In one specific embodiment, the crystalline Form C of the pamoate salt solvate exhibits an XRPD pattern that is substantially similar to FIG. 3A.

In one embodiment, the crystalline Form C of the pamoate salt solvate exhibits a DSC thermogram comprising a desolvation peak at about 37.3° C. (onset) with the error of margin of about ±2.5; about ±2.0; about ±1.5; about ±1.0; about ±0.5; or less. In one embodiment, the enthalpy of desolvation is about 76 J/g by DSC. In one embodiment, the crystalline Form C exhibits a DSC thermogram comprising an exotherm peak at about 170° C. (onset) with the error of margin of about ±2.5; about ±2.0; about ±1.5; about ±1.0; about ±0.5; or less. In one embodiment, the crystalline Form C exhibits a DSC thermogram comprising an exotherm peak at about 173° C. (peak maximum) with the error of margin of about ±2.5; about ±2.0; about ±1.5; about ±1.0; about ±0.5; or less. In one embodiment, the enthalpy of the exothermic peak is about 20 J/g by DSC. In one embodiment, the crystalline Form C exhibits a DSC thermogram comprising an endotherm peak at about 238° C. (onset) with the error of margin of about ±2.5; about ±2.0; about ±1.5; about ±1.0; about ±0.5; or less. In one embodiment, the crystalline Form C exhibits a DSC thermogram comprising an endotherm peak at about 241° C. (peak maximum) with the error of margin of about ±2.5; about ±2.0; about ±1.5; about ±1.0; about ±0.5; or less. In one embodiment, the crystalline Form C of the pamoate salt exhibits a DSC thermogram that is substantially similar to FIG. 3B.

In one embodiment, the crystalline Form C of the pamoate salt solvate exhibits a TGA thermogram substantially similar to FIG. 3C. In one embodiment, crystalline Form C shows about 7.2% weight loss in the range of about 35° C. to about 200° C. by TGA.

### Form D

In one embodiment, the present disclosure relates to a crystalline Form D of the pamoate salt solvate. In one embodiment, Form D of the pamoate salt is an N-methyl pyrrolidone (NMP) solvate. In one embodiment, Form D is of high crystallinity. In one embodiment, Form D has an imidazolium:pamoic acid stoichiometry of about 1:1. In one embodiment, Form D comprises NMP in about 9 equiv. as determined by ¹H NMR. In one embodiment, Form D comprises NMP in about 54.2% by weight.

In one embodiment, Form D of the pamoate salt solvate may comprise of a mixture of one or more forms of the pamoate salt or solvate thereof. In some embodiments, the crystalline form of the pamoate salt or solvate thereof may comprise of substantially pure form of Form D. In one embodiment, the crystalline form of the pamoate salt or solvate thereof may comprise of over about 99.9%, about 99.8%, about 99.7%, about 99.6%, about 99.5%, about 99.4%, about 99.3%, about 99.2%, about 99.1%, or about 99.0% of Form D. In another embodiment, the crystalline form of the pamoate salt or solvate thereof may comprise over about 99%, 98%, 97%, 96%, 95%, 94%, 93%, 92%, 91%, or 90% of Form D. In some embodiments, the crystalline form of the pamoate salt or solvate thereof may comprise over about 90%, 85%, 80%, 75%, 70%, 65%, 60%, 55%, 50%, 45%, or 40% of Form D.

In one embodiment, Form D of the pamoate salt solvate comprises one or both of Form A and Form B of the pamoate salt.

In one embodiment, Form D of the pamoate salt solvate is isolated and purified. In one embodiment, the purified Form D of the pamoate salt solvate is at least 95%, at least 96%, at least 97%, at least 98%, at least 99%, or at least 99.5% pure.

In one embodiment, crystalline Form D of the pamoate salt solvate exhibits an XRPD pattern comprising peaks at about 7.46, 20.27, and 24.93 degrees two-theta with the margin of error of about ±0.5; about ±0.4; about ±0.3; about ±0.2; about ±0.1; about ±0.05; or less. In another embodiment, the XRPD of the crystalline Form D further comprises peaks at about 21.05, 22.44, and 24.57 degrees two-theta with the margin of error of about ±0.5; about ±0.4; about ±0.3; about ±0.2; about ±0.1; about ±0.05; or less. In further embodiment, the crystalline Form D further comprises peaks at about 9.00 and 17.77 degrees two-theta with the margin of error of about ±0.5; about ±0.4; about ±0.3; about 0.2; about ±0.1; about ±0.05; or less.

In one embodiment, crystalline Form D of the pamoate salt solvate exhibits an XRPD pattern comprising peaks at about 7.46±0.2, 20.27±0.2, and 24.93±0.2 degrees two-theta. In one embodiment, crystalline Form D exhibits an XRPD pattern comprising three of the ten most intense peaks at about 7.46±0.2, 20.27±0.2, and 24.93±0.2 degrees two-theta. In one embodiment, crystalline Form D exhibits an XRPD pattern comprising three of the six most intense peaks at about 7.46±0.2, 20.27±0.2, and 24.93±0.2 degrees two-theta.

In one embodiment, crystalline Form D of the pamoate salt solvate exhibits an XRPD pattern comprising peaks at about 21.05±0.2, 22.44±0.2, and 24.57±0.2 degrees two-theta. In one embodiment, crystalline Form D exhibits an XRPD pattern comprising three of the ten most intense peaks at about 21.05±0.2, 22.44±0.2, and 24.57±0.2 degrees two-theta. In one embodiment, crystalline Form D exhibits an XRPD pattern comprising three of the six most intense peaks at about 21.05±0.2, 22.44±0.2, and 24.57±0.2 degrees two-theta.

In one embodiment, crystalline Form D of the pamoate salt solvate exhibits an XRPD pattern comprising at least two peaks selected from about 7.46±0.2, 20.27±0.2, 21.05±0.2, 22.44±0.2, 24.57±0.2, or 24.93±0.2 degrees two-theta. In one embodiment, crystalline Form D of the pamoate salt solvate exhibits an XRPD pattern comprising at least three peaks selected from about 7.46±0.2, 20.27±0.2, 21.05±0.2, 22.44±0.2, 24.57±0.2, or 24.93±0.2 degrees two-theta. In one embodiment, crystalline Form D of the pamoate salt solvate exhibits an XRPD pattern comprising at least four peaks selected from about 7.46±0.2, 20.27±0.2, 21.05±0.2, 22.44±0.2, 24.57±0.2, or 24.93±0.2 degrees two-theta. In one embodiment, crystalline Form D of the pamoate salt solvate exhibits an XRPD pattern comprising at least five peaks selected from about 7.46±0.2, 20.27±0.2, 21.05±0.2, 22.44±0.2, 24.57±0.2, or 24.93±0.2 degrees two-theta. In one embodiment, crystalline Form D of the pamoate salt solvate exhibits an XRPD pattern comprising peaks at about 7.46±0.2, 20.27±0.2, 21.05±0.2, 22.44±0.2, 24.57±0.2, and 24.93±0.2 degrees two-theta.

In one embodiment, crystalline Form D of the pamoate salt solvate exhibits an XRPD pattern comprising at least two peaks, at least three peaks, at least four peaks, at least five peaks, at least six peaks, at least seven peaks, at least eight peaks, or at least nine peaks selected from about 7.46±0.2, 9.00±0.2, 9.41±0.2, 17.77±0.2, 20.27±0.2, 21.05±0.2, 22.27±0.2, 22.44±0.2, 24.57±0.2, or 24.93±0.2 degrees two-theta. In one embodiment, crystalline Form D of the pamoate salt solvate exhibits an XRPD pattern comprising peaks at about 7.46±0.2, 9.00±0.2, 9.41±0.2, 17.77±0.2, 20.27±0.2, 21.05±0.2, 22.27±0.2, 22.44±0.2, 24.57±0.2, and 24.93±0.2 degrees two-theta.

In one embodiment, the crystalline Form D of the pamoate salt solvate exhibits an XRPD comprising peaks shown in Table 4 below. In one embodiment, the crystalline Form D of the pamoate salt exhibits an XRPD comprising at least five most intense peaks shown in Table 4 below (net intensity). In one embodiment, the crystalline Form D of the pamoate salt exhibits an XRPD comprising at least six, at least seven, at least eight, at least nine, at least ten most intense peaks shown in Table 4 below (net intensity).

TABLE 4
XRPD Table of Form D of the pamoate salt solvate

| Angle (°2θ) | D Value (Å) | Net Intensity | Gross Intensity |
|---|---|---|---|
| 6.607 | 13.36817 | 30.5534 | 85.1145 |
| 7.460 | 11.84148 | 3032.89 | 3091.94 |
| 9.003 | 9.81512 | 340.280 | 403.264 |
| 9.247 | 9.55580 | 88.0553 | 150.792 |
| 9.414 | 9.38663 | 284.583 | 346.685 |
| 10.499 | 8.41885 | 201.977 | 265.039 |
| 10.615 | 8.32777 | 113.452 | 176.845 |
| 11.230 | 7.87251 | 48.5030 | 110.589 |
| 11.997 | 7.37091 | 31.7953 | 98.4967 |
| 12.958 | 6.82675 | 238.634 | 305.930 |
| 13.874 | 6.37769 | 47.0315 | 116.809 |
| 14.244 | 6.21293 | 89.9625 | 161.694 |
| 15.000 | 5.90158 | 104.686 | 174.515 |
| 15.131 | 5.85086 | 106.115 | 174.829 |
| 16.719 | 5.29833 | 81.1643 | 149.625 |
| 17.095 | 5.18258 | 280.499 | 351.936 |
| 17.769 | 4.98756 | 288.330 | 363.931 |
| 18.143 | 4.88568 | 220.006 | 297.398 |
| 18.467 | 4.80060 | 67.1456 | 144.563 |
| 18.867 | 4.69973 | 281.444 | 358.138 |
| 19.577 | 4.53078 | 142.216 | 222.446 |
| 19.816 | 4.47664 | 156.579 | 240.189 |
| 20.272 | 4.37703 | 491.248 | 579.151 |
| 20.694 | 4.28873 | 116.303 | 205.673 |
| 21.051 | 4.21686 | 342.137 | 430.867 |
| 21.663 | 4.09910 | 110.928 | 194.537 |
| 22.269 | 3.98890 | 284.216 | 364.824 |
| 22.440 | 3.95886 | 424.319 | 503.848 |
| 24.162 | 3.68046 | 90.8466 | 166.716 |
| 24.573 | 3.61984 | 363.908 | 447.858 |
| 24.931 | 3.56868 | 511.738 | 600.863 |
| 25.568 | 3.48120 | 255.520 | 349.568 |
| 26.074 | 3.41472 | 168.204 | 262.237 |
| 26.384 | 3.37530 | 103.950 | 196.257 |
| 26.920 | 3.30933 | 47.2046 | 136.985 |
| 27.438 | 3.24799 | 146.247 | 237.806 |
| 27.822 | 3.20402 | 194.027 | 284.556 |
| 28.286 | 3.15257 | 59.4832 | 146.105 |
| 29.416 | 3.03400 | 34.7220 | 104.925 |
| 29.817 | 2.99409 | 62.1550 | 131.881 |
| 31.598 | 2.82920 | 250.432 | 323.847 |
| 33.161 | 2.69935 | 35.9877 | 96.4123 |
| 33.540 | 2.66971 | 46.1552 | 111.666 |

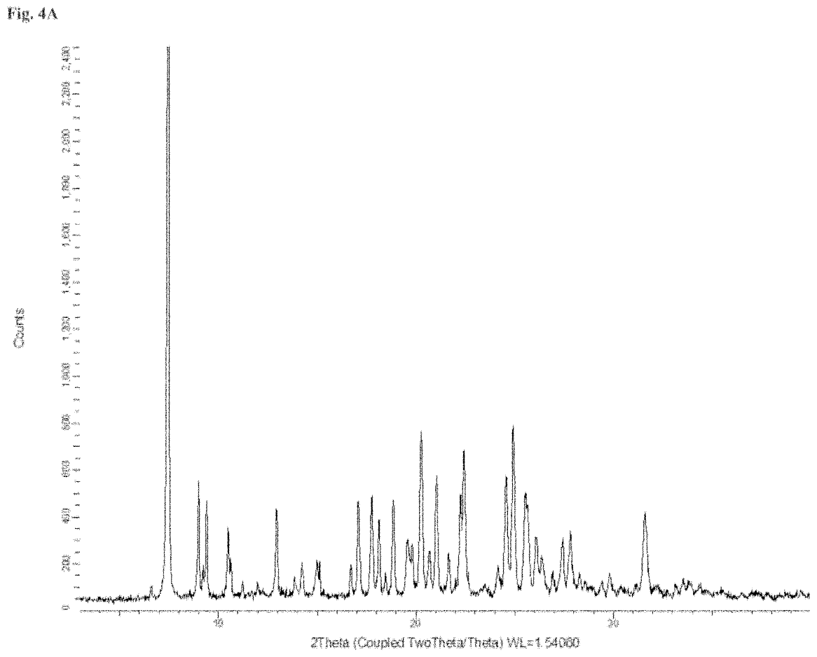

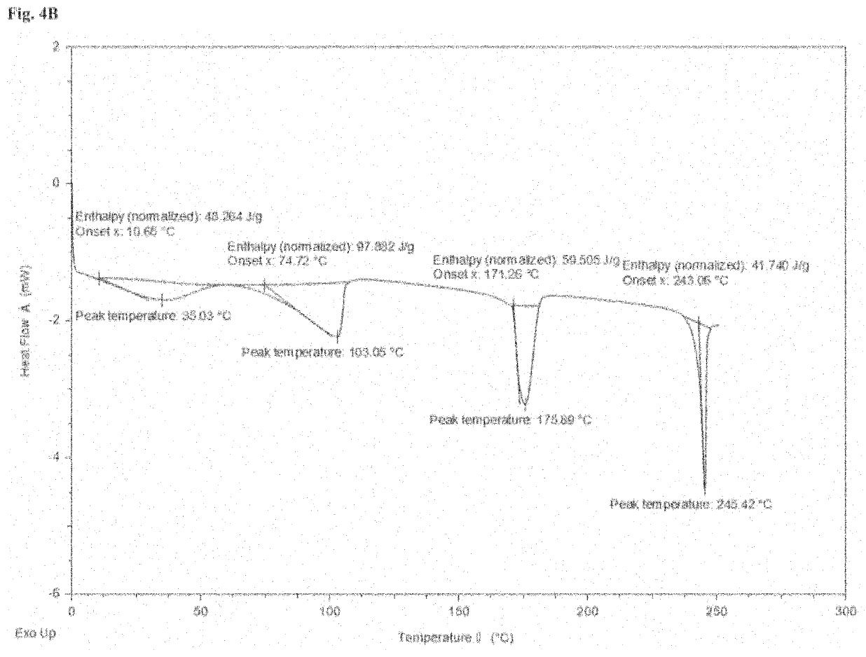

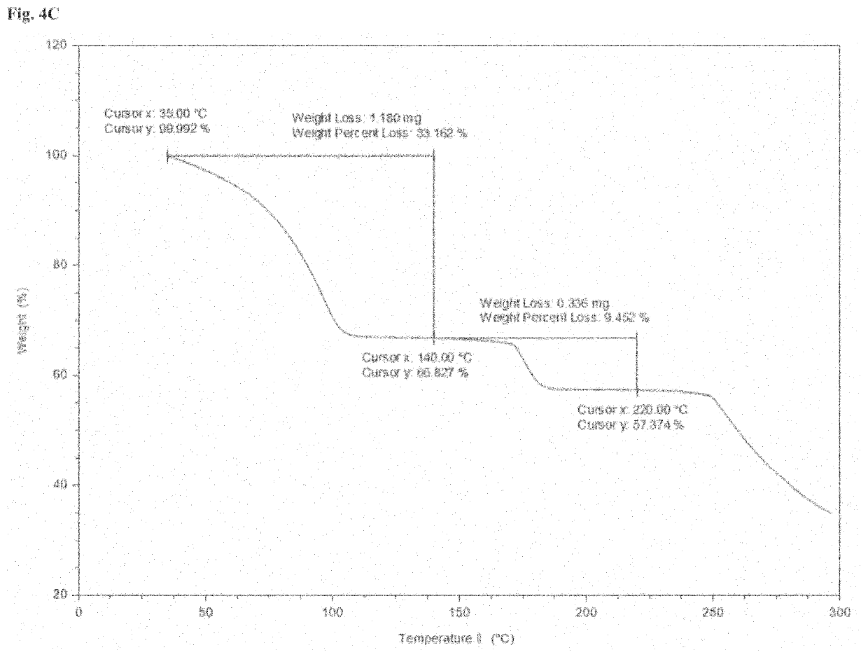

In one specific embodiment, the crystalline Form D of the pamoate salt solvate exhibits an XRPD pattern that is substantially similar to FIG. 4A.

In one embodiment, the crystalline Form D of the pamoate salt solvate exhibits a DSC thermogram comprising a desolvation peak at about 11° C. (onset) with the error of margin of about ±2.5; about ±2.0; about ±1.5; about ±1.0; about ±0.5; or less. In one embodiment, the enthalpy of desolvation is about 48 J/g for the peak at about 11° C. (onset) by DSC. In one embodiment, the crystalline Form D exhibits a DSC thermogram comprising a desolvation peak at about 75° C. (onset) with the error of margin of about ±2.5; about ±2.0; about ±1.5; about ±1.0; about ±0.5; or less. In one embodiment, the enthalpy of desolvation is about 98 J/g for the peak at about 75° C. (onset) by DSC. In one embodiment, the crystalline Form D of the pamoate salt solvate exhibits a DSC thermogram comprising a desolvation peak at about 171° C. (onset) with the error of margin of about ±2.5; about ±2.0; about ±1.5; about ±1.0; about ±0.5; or less. In one embodiment, the enthalpy of desolvation is about 60 J/g for the peak at about 171° C. (onset) by DSC. In one embodiment, the crystalline Form D exhibits a DSC thermogram comprising an endotherm peak at about 243° C. (onset) with the error of margin of about ±2.5; about ±2.0; about ±1.5; about ±1.0; about ±0.5; or less. In one embodiment, the crystalline Form D exhibits a DSC thermogram comprising an endotherm peak at about 245° C. (peak maximum) with the error of margin of about ±2.5; about ±2.0; about ±1.5; about ±1.0; about ±0.5; or less. In one embodiment, the crystalline Form D of the pamoate salt exhibits a DSC thermogram that is substantially similar to FIG. 4B.

In one embodiment, the crystalline Form D of the pamoate salt solvate exhibits a TGA thermogram substantially similar to FIG. 4C. In one embodiment, crystalline Form D shows about 33.2% weight loss in the range of about 35° C. to about 140° C. by TGA. In one embodiment, crystalline Form D shows about 9.5% weight loss in the range of about 140° C. to about 220° C. by TGA.

### Form E

In one embodiment, the present disclosure relates to a crystalline Form E of the pamoate salt solvate. In one embodiment, Form E of the pamoate salt is an N,N-dimethylacetamide (DMAc) solvate. In one embodiment, Form E is of low crystallinity. In one embodiment, Form E has an imidazolium:pamoic acid stoichiometry of about 1:1.1. In one embodiment, Form E comprises DMAc in about 1.6 equiv. as determined by ¹H NMR. In one embodiment, Form E comprises DMAc in about 15.7% by weight.

In one embodiment, Form E of the pamoate salt solvate may comprise of a mixture of one or more forms of the pamoate salt or solvate thereof. In some embodiments, the crystalline form of the pamoate salt or solvate thereof may comprise of substantially pure form of Form E. In one embodiment, the crystalline form of the pamoate salt or solvate thereof may comprise of over about 99.9%, about 99.8%, about 99.7%, about 99.6%, about 99.5%, about 99.4%, about 99.3%, about 99.2%, about 99.1%, or about 99.0% of Form E. In another embodiment, the crystalline form of the pamoate salt or solvate thereof may comprise over about 99%, 98%, 97%, 96%, 95%, 94%, 93%, 92%, 91%, or 90% of Form E. In some embodiments, the crystalline form of the pamoate salt or solvate thereof may comprise over about 90%, 85%, 80%, 75%, 70%, 65%, 60%, 55%, 50%, 45%, or 40% of Form E.

In one embodiment, Form E of the pamoate salt solvate comprises one or both of Form A and Form B of the pamoate salt.

In one embodiment, Form E of the pamoate salt solvate is isolated and purified. In one embodiment, the purified Form E of the pamoate salt solvate is at least 95%, at least 96%, at least 97%, at least 98%, at least 99%, or at least 99.5% pure.

In one embodiment, crystalline Form E of the pamoate salt solvate exhibits an XRPD pattern comprising peaks at about 6.81, 8.31, and 24.60 degrees two-theta with the margin of error of about ±0.5; about ±0.4; about ±0.3; about ±0.2; about ±0.1; about ±0.05; or less. In another embodiment, the XRPD of the crystalline Form E further comprises peaks at about 25.16 and 25.84 degrees two-theta with the margin of error of about ±0.5; about ±0.4; about ±0.3; about ±0.2; about 0.1; about ±0.05; or less.

In one embodiment, crystalline Form E of the pamoate salt solvate exhibits an XRPD pattern comprising peaks at about 6.81±0.2, 8.31±0.2, and 24.60±0.2 degrees two-theta. In one embodiment, crystalline Form E exhibits an XRPD pattern comprising three of the ten most intense peaks at about 6.81±0.2, 8.31±0.2, and 24.60±0.2 degrees two-theta. In one embodiment, crystalline Form E exhibits an XRPD pattern comprising three of the five most intense peaks at about 6.81±0.2, 8.31±0.2, and 24.60±0.2 degrees two-theta.

In one embodiment, crystalline Form E of the pamoate salt solvate exhibits an XRPD pattern comprising peaks at about 25.16±0.2 and 25.84±0.2 degrees two-theta. In one embodiment, crystalline Form E exhibits an XRPD pattern comprising two of the ten most intense peaks at about 25.16±0.2 and 25.84±0.2 degrees two-theta. In one embodiment, crystalline Form E exhibits an XRPD pattern comprising two of the five most intense peaks at about 25.16±0.2 and 25.84±0.2 degrees two-theta.

In one embodiment, crystalline Form E of the pamoate salt solvate exhibits an XRPD pattern comprising at least two peaks selected from about 6.81±0.2, 8.31±0.2, 24.60±0.2, 25.16±0.2 or 25.84±0.2 degrees two-theta. In one embodiment, crystalline Form E of the pamoate salt solvate exhibits an XRPD pattern comprising at least three peaks selected from about 6.81±0.2, 8.31±0.2, 24.60±0.2, 25.16±0.2 or 25.84±0.2 degrees two-theta. In one embodiment, crystalline Form E of the pamoate salt solvate exhibits an XRPD pattern comprising at least four peaks selected from about 6.81±0.2, 8.31±0.2, 24.60±0.2, 25.16±0.2 or 25.84±0.2 degrees two-theta. In one embodiment, crystalline Form E of the pamoate salt solvate exhibits an XRPD pattern comprising peaks at about 6.81±0.2, 8.31±0.2, 24.60±0.2, 25.16±0.2 and 25.84±0.2 degrees two-theta.

In one embodiment, the crystalline Form E of the pamoate salt solvate exhibits an XRPD comprising peaks shown in Table 5 below. In one embodiment, the crystalline Form E of the pamoate salt exhibits an XRPD comprising at least five most intense peaks shown in Table 5 below (net intensity).

TABLE 5
XRPD Table of Form E of the pamoate salt solvate

| Angle (°2θ) | D Value (Å) | Net Intensity | Gross Intensity |
|---|---|---|---|
| 6.809 | 12.97148 | 46.6862 | 89.7874 |
| 8.313 | 10.62772 | 137.992 | 185.513 |
| 11.966 | 7.39041 | 19.7018 | 73.0394 |
| 12.493 | 7.07983 | 20.5647 | 74.7130 |
| 18.196 | 4.87146 | 19.9931 | 59.4115 |
| 20.315 | 4.36793 | 21.6760 | 59.8644 |
| 20.795 | 4.26811 | 28.0114 | 65.7844 |
| 22.570 | 3.93642 | 15.4583 | 54.0976 |
| 24.603 | 3.61545 | 51.0048 | 92.9534 |
| 25.162 | 3.53643 | 38.8485 | 80.1217 |
| 25.838 | 3.44546 | 37.6561 | 76.6969 |
| 28.430 | 3.13693 | 14.4731 | 54.2832 |
| 29.456 | 3.02988 | 13.6169 | 52.6600 |

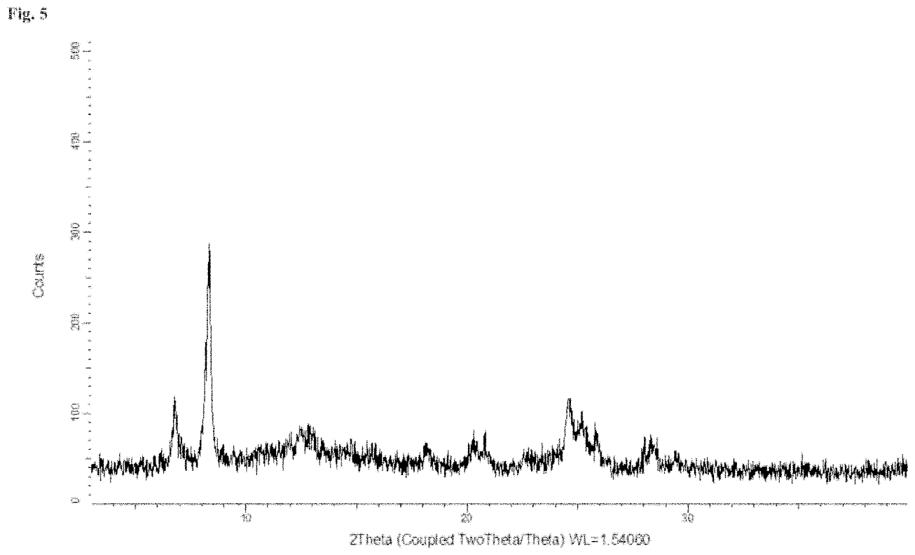

In one specific embodiment, the crystalline Form E of the pamoate salt solvate exhibits an XRPD pattern that is substantially similar to FIG. 5.

### Form F

In one embodiment, the present disclosure relates to a crystalline Form F of the pamoate salt solvate. In one embodiment, Form F of the pamoate salt is an N,N-dimethylacetamide (DMAc) solvate. In one embodiment, Form F is of medium crystallinity. In one embodiment, Form F has an imidazolium:pamoic acid stoichiometry of about 1:1. In one embodiment, Form F comprises DMAc in about 14.6 equiv. as determined by ¹H NMR. In one embodiment, Form F comprises DMAc in about 62.9% by weight.

In one embodiment, Form F of the pamoate salt solvate may comprise of a mixture of one or more forms of the pamoate salt or solvate thereof. In some embodiments, the crystalline form of the pamoate salt or solvate thereof may comprise of substantially pure form of Form F. In one embodiment, the crystalline form of the pamoate salt or solvate thereof may comprise of over about 99.9%, about 99.8%, about 99.7%, about 99.6%, about 99.5%, about 99.4%, about 99.3%, about 99.2%, about 99.10%, or about 99.0% of Form F. In another embodiment, the crystalline form of the pamoate salt or solvate thereof may comprise over about 99%, 98%, 97%, 96%, 95%, 94%, 93%, 92%, 91%, or 90% of Form F. In some embodiments, the crystalline form of the pamoate salt or solvate thereof may comprise over about 90%, 85%, 80%, 75%, 70%, 65%, 60%, 55%, 50%, 45%, or 40% of Form F.

In one embodiment, Form F of the pamoate salt solvate comprises one or both of Form A and Form B of the pamoate salt.

In one embodiment, Form F of the pamoate salt solvate is isolated and purified. In one embodiment, the purified Form F of the pamoate salt solvate is at least 95%, at least 96%, at least 97%, at least 98%, at least 99%, or at least 99.5% pure.

In one embodiment, crystalline Form F of the pamoate salt solvate exhibits an XRPD pattern comprising peaks at about 11.44, 13.81, and 24.55 degrees two-theta with the margin of error of about 0.5; about 0.4; about 0.3; about 0.2; about 0.1; about 0.05; or less. In another embodiment, the XRPD of the crystalline Form F further comprises peaks at about 6.91, 12.21, and 22.70 degrees two-theta with the margin of error of about ±0.5; about ±0.4; about ±0.3; about ±0.2; about ±0.1; about ±0.05; or less. In further embodiment, the crystalline Form F further comprises peaks at about 18.97 and 25.37 degrees two-theta with the margin of error of about ±0.5; about ±0.4; about ±0.3; about ±0.2; about ±0.1; about ±0.05; or less.

In one embodiment, crystalline Form F of the pamoate salt solvate exhibits an XRPD pattern comprising peaks at about 11.44±0.2, 13.81±0.2, and 24.55±0.2 degrees two-theta. In one embodiment, crystalline Form F exhibits an XRPD pattern comprising three of the ten most intense peaks at about 11.44±0.2, 13.81±0.2, and 24.55±0.2 degrees two-theta. In one embodiment, crystalline Form F exhibits an XRPD pattern comprising three of the six most intense peaks at about 11.44±0.2, 13.81±0.2, and 24.55±0.2 degrees two-theta.

In one embodiment, crystalline Form F of the pamoate salt solvate exhibits an XRPD pattern comprising peaks at about 6.91±0.2, 12.21±0.2, and 22.70±0.2 degrees two-theta. In one embodiment, crystalline Form F exhibits an XRPD pattern comprising three of the ten most intense peaks at about 6.91±0.2, 12.21±0.2, and 22.70±0.2 degrees two-theta. In one embodiment, crystalline Form F exhibits an XRPD pattern comprising three of the six most intense peaks at about 6.91±0.2, 12.21±0.2, and 22.70±0.2 degrees two-theta.

In one embodiment, crystalline Form F of the pamoate salt solvate exhibits an XRPD pattern comprising at least two peaks selected from about 6.91±0.2, 11.44±0.2, 12.21±0.2, 13.81±0.2, 22.70±0.2, or 24.55±0.2 degrees two-theta. In one embodiment, crystalline Form F of the pamoate salt solvate exhibits an XRPD pattern comprising at least three peaks selected from about 6.91±0.2, 11.44±0.2, 12.21±0.2, 13.81±0.2, 22.70±0.2, or 24.55±0.2 degrees two-theta. In one embodiment, crystalline Form F of the pamoate salt solvate exhibits an XRPD pattern comprising at least four peaks selected from about 6.91±0.2, 11.44±0.2, 12.21±0.2, 13.81±0.2, 22.70±0.2, or 24.55±0.2 degrees two-theta. In one embodiment, crystalline Form F of the pamoate salt solvate exhibits an XRPD pattern comprising at least five peaks selected from about 6.91±0.2, 11.44±0.2, 12.21±0.2, 13.81±0.2, 22.70±0.2, or 24.55±0.2 degrees two-theta. In one embodiment, crystalline Form F of the pamoate salt solvate exhibits an XRPD pattern comprising peaks at about 6.91±0.2, 11.44±0.2, 12.21±0.2, 13.81±0.2, 22.70±0.2, and 24.55±0.2 degrees two-theta.

In one embodiment, crystalline Form F of the pamoate salt solvate exhibits an XRPD pattern comprising at least two peaks, at least three peaks, at least four peaks, at least five peaks, at least six peaks, at least seven peaks, at least eight peaks, or at least nine peaks selected from about 6.91±0.2, 11.44±0.2, 12.21±0.2, 13.81±0.2, 18.97±0.2, 20.77±0.2, 22.70±0.2, 24.55±0.2, 25.37±0.2, or 26.18±0.2 degrees two-theta. In one embodiment, crystalline Form F of the pamoate salt solvate exhibits an XRPD pattern comprising peaks at about 6.91±0.2, 11.44±0.2, 12.21±0.2, 13.81±0.2, 18.97±0.2, 20.77±0.2, 22.70±0.2, 24.55±0.2, 25.37±0.2, and 26.18±0.2 degrees two-theta.

In one embodiment, the crystalline Form F of the pamoate salt solvate exhibits an XRPD comprising peaks shown in Table 6 below. In one embodiment, the crystalline Form F of the pamoate salt exhibits an XRPD comprising at least five most intense peaks shown in Table 6 below (net intensity). In one embodiment, the crystalline Form F of the pamoate salt exhibits an XRPD comprising at least six, at least seven, at least eight, at least nine, at least ten most intense peaks shown in Table 6 below (net intensity).

TABLE 6
XRPD Table of Form F of the pamoate salt solvate

| Angle (°2θ) | D Value (Å) | Net Intensity | Gross Intensity |
|---|---|---|---|
| 6.912 | 12.77743 | 164.416 | 217.459 |
| 9.423 | 9.37836 | 77.3816 | 136.744 |
| 11.437 | 7.73044 | 252.558 | 324.472 |
| 12.209 | 7.24346 | 156.181 | 234.714 |
| 13.808 | 6.40831 | 382.170 | 464.745 |
| 14.830 | 5.96873 | 29.1699 | 107.493 |
| 15.224 | 5.81515 | 24.5449 | 99.8066 |
| 15.938 | 5.55606 | 20.9838 | 92.3698 |
| 16.547 | 5.35295 | 33.3105 | 102.196 |
| 17.740 | 4.99557 | 54.1691 | 121.896 |
| 18.974 | 4.67341 | 149.516 | 225.985 |
| 19.538 | 4.53982 | 73.8713 | 151.749 |
| 20.049 | 4.42531 | 40.7416 | 118.495 |
| 20.770 | 4.27319 | 118.981 | 195.630 |
| 21.641 | 4.10310 | 15.1080 | 87.5564 |
| 22.139 | 4.01200 | 39.2580 | 110.934 |
| 22.699 | 3.91430 | 196.299 | 269.580 |
| 24.545 | 3.62393 | 403.529 | 484.174 |
| 25.371 | 3.50779 | 125.428 | 207.309 |
| 26.182 | 3.40090 | 117.302 | 197.010 |
| 27.250 | 3.27001 | 46.4891 | 120.895 |
| 28.369 | 3.14352 | 36.8815 | 102.216 |
| 31.306 | 2.85500 | 24.6865 | 76.3901 |
| 32.008 | 2.79396 | 39.1301 | 93.4225 |

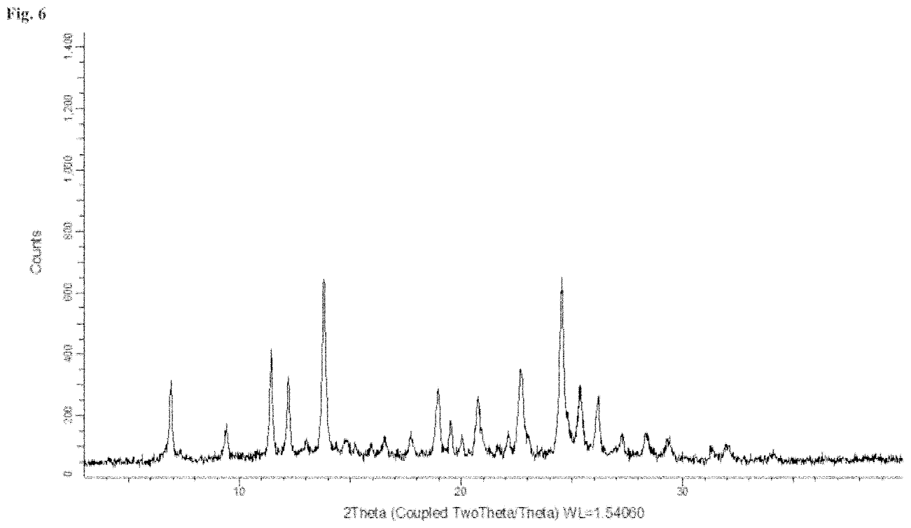

In one specific embodiment, the crystalline Form F of the pamoate salt solvate exhibits an XRPD pattern that is substantially similar to FIG. 6.

### Form G

In one embodiment, the present disclosure relates to a crystalline Form G of the pamoate salt solvate. In one embodiment, Form G of the pamoate salt is an N,N-dimethylacetamide (DMAc) solvate. In one embodiment, Form G is of high crystallinity.

In one embodiment, Form G of the pamoate salt solvate may comprise of a mixture of one or more forms of the pamoate salt or solvate thereof. In some embodiments, the crystalline form of the pamoate salt or solvate thereof may comprise of substantially pure form of Form G. In one embodiment, the crystalline form of the pamoate salt or solvate thereof may comprise of over about 99.9%, about 99.8%, about 99.7%, about 99.6%, about 99.5%, about 99.4%, about 99.3%, about 99.2%, about 99.1%, or about 99.0% of Form G. In another embodiment, the crystalline form of the pamoate salt or solvate thereof may comprise over about 99%, 98%, 97%, 96%, 95%, 94%, 93%, 92%, 91%, or 90% of Form G. In some embodiments, the crystalline form of the pamoate salt or solvate thereof may comprise over about 90%, 85%, 80%, 75%, 70%, 65%, 60%, 55%, 50%, 45%, or 40% of Form G.

In one embodiment, Form G of the pamoate salt solvate comprises one or more of Form A Form B, and Form H of the pamoate salt or solvate. In one embodiment, Form G comprises Form H.

In one embodiment, Form G of the pamoate salt solvate is isolated and purified. In one embodiment, the purified Form G of the pamoate salt solvate is at least 95%, at least 96%, at least 97%, at least 98%, at least 99%, or at least 99.5% pure.

In one embodiment, crystalline Form G of the pamoate salt solvate exhibits an XRPD pattern comprising peaks at about 18.86 and 23.51 degrees two-theta with the margin of error of about ±0.5; about ±0.4; about ±0.3; about ±0.2; about ±0.1; about ±0.05; or less. In another embodiment, the XRPD of the crystalline Form G further comprises peaks at about 17.38, 20.54, and 21.24 degrees two-theta with the margin of error of about ±0.5; about ±0.4; about ±0.3; about ±0.2; about ±0.1; about ±0.05; or less. In further embodiment, the crystalline Form G further comprises at least two peaks selected from about 14.73, 19.95, 21.45, or 29.59 degrees two-theta with the margin of error of about ±0.5; about ±0.4; about ±0.3; about ±0.2; about ±0.1; about ±0.05; or less.

In one embodiment, crystalline Form G of the pamoate salt solvate exhibits an XRPD pattern comprising peaks at about 18.86±0.2 and 23.51±0.2 degrees two-theta. In one embodiment, crystalline Form G exhibits an XRPD pattern comprising two of the ten most intense peaks at about 18.86±0.2 and 23.51±0.2 degrees two-theta. In one embodiment, crystalline Form G exhibits an XRPD pattern comprising two of the five most intense peaks at about 18.86±0.2 and 23.51±0.2 degrees two-theta.

In one embodiment, crystalline Form G of the pamoate salt solvate exhibits an XRPD pattern comprising peaks at about 17.38±0.2, 20.54±0.2, and 21.24±0.2 degrees two-theta. In one embodiment, crystalline Form G exhibits an XRPD pattern comprising three of the ten most intense peaks at about 17.38±0.2, 20.54±0.2, and 21.24±0.2 degrees two-theta. In one embodiment, crystalline Form G exhibits an XRPD pattern comprising three of the five most intense peaks at about 17.38±0.2, 20.54±0.2, and 21.24±0.2 degrees two-theta.

In one embodiment, crystalline Form G of the pamoate salt solvate exhibits an XRPD pattern comprising at least two peaks selected from about 17.38±0.2, 18.86±0.2, 20.54±0.2, 21.24±0.2, or 23.51±0.2 degrees two-theta. In one embodiment, crystalline Form G of the pamoate salt solvate exhibits an XRPD pattern comprising at least three peaks selected from about 17.38±0.2, 18.86±0.2, 20.54±0.2, 21.24±0.2, or 23.51±0.2 degrees two-theta. In one embodiment, crystalline Form G of the pamoate salt solvate exhibits an XRPD pattern comprising at least four peaks selected from about 17.38±0.2, 18.86±0.2, 20.54±0.2, 21.24±0.2, or 23.51±0.2 degrees two-theta. In one embodiment, crystalline Form G of the pamoate salt solvate exhibits an XRPD pattern comprising peaks at about 17.38±0.2, 18.86±0.2, 20.54±0.2, 21.24±0.2, and 23.51±0.2 degrees two-theta.

In one embodiment, crystalline Form G of the pamoate salt solvate exhibits an XRPD pattern comprising at least two peaks, at least three peaks, at least four peaks, at least five peaks, at least six peaks, at least seven peaks, at least eight peaks, or at least nine peaks selected from about 14.73±0.2, 17.38±0.2, 18.86±0.2, 19.95±0.2, 20.54±0.2, 21.24±0.2, 21.45±0.2, 23.51±0.2 or 29.59±0.2 degrees two-theta. In one embodiment, crystalline Form G of the pamoate salt solvate exhibits an XRPD pattern comprising peaks at about 14.73±0.2, 17.38±0.2, 18.86±0.2, 19.95±0.2, 20.54±0.2, 21.24±0.2, 21.45±0.2, 23.51±0.2 and 29.59±0.2 degrees two-theta.

In one embodiment, the crystalline Form G of the pamoate salt solvate exhibits an XRPD comprising peaks shown in Table 7 below. In one embodiment, the crystalline Form G of the pamoate salt exhibits an XRPD comprising at least five most intense peaks shown in Table 7 below (net intensity). In one embodiment, the crystalline Form G of the pamoate salt exhibits an XRPD comprising at least six, at least seven, at least eight, at least nine, at least ten most intense peaks shown in Table 7 below (net intensity).

TABLE 7
XRPD Table of Form G of the pamoate salt solvate

| Angle (°2θ) | D Value (Å) | Net Intensity | Gross Intensity |
|---|---|---|---|
| 7.897 | 11.18672 | 100.764 | 168.687 |
| 9.517 | 9.28559 | 98.5484 | 190.483 |
| 10.565 | 8.36665 | 186.336 | 302.161 |
| 11.059 | 7.99435 | 71.6642 | 198.133 |
| 11.546 | 7.65813 | 53.8204 | 189.031 |
| 12.048 | 7.34017 | 41.9363 | 183.724 |
| 13.774 | 6.42390 | 361.219 | 542.466 |
| 14.726 | 6.01072 | 901.701 | 1113.37 |
| 16.905 | 5.24039 | 644.804 | 882.812 |
| 17.380 | 5.09848 | 1197.13 | 1432.88 |
| 18.064 | 4.90687 | 27.5657 | 255.036 |
| 18.855 | 4.70272 | 2629.47 | 2875.75 |
| 19.227 | 4.61249 | 336.896 | 598.749 |
| 19.950 | 4.44696 | 852.619 | 1139.68 |
| 20.535 | 4.32169 | 1685.94 | 1988.53 |
| 21.243 | 4.17915 | 1550.59 | 1866.19 |
| 21.448 | 4.13970 | 848.018 | 1166.20 |
| 21.817 | 4.07044 | 689.516 | 1010.99 |
| 22.688 | 3.91619 | 407.265 | 729.667 |
| 22.729 | 3.90916 | 402.076 | 724.283 |
| 23.506 | 3.78174 | 2360.42 | 2674.93 |
| 24.826 | 3.58352 | 300.569 | 584.418 |
| 25.236 | 3.52623 | 419.159 | 688.989 |
| 26.074 | 3.41473 | 248.818 | 500.623 |
| 26.680 | 3.33851 | 171.926 | 418.966 |
| 27.088 | 3.28921 | 367.240 | 608.456 |
| 27.635 | 3.22534 | 245.467 | 481.237 |
| 28.242 | 3.15732 | 774.068 | 1007.25 |
| 28.709 | 3.10706 | 116.596 | 344.605 |
| 29.587 | 3.01680 | 851.906 | 1081.44 |
| 30.440 | 2.93416 | 59.1311 | 295.969 |
| 30.677 | 2.91203 | 143.677 | 380.903 |
| 31.208 | 2.86368 | 47.0948 | 282.916 |
| 31.497 | 2.83805 | 194.564 | 428.443 |
| 31.828 | 2.80932 | 221.648 | 451.998 |
| 32.785 | 2.72947 | 312.058 | 538.805 |
| 33.250 | 2.69232 | 431.310 | 674.181 |
| 33.696 | 2.65773 | 538.441 | 794.175 |
| 34.027 | 2.63267 | 643.200 | 906.849 |
| 34.439 | 2.60206 | 530.303 | 801.881 |
| 35.036 | 2.55909 | 210.808 | 490.036 |
| 35.648 | 2.51655 | 223.962 | 506.341 |
| 36.222 | 2.47796 | 532.487 | 813.500 |
| 36.771 | 2.44224 | 241.062 | 516.860 |
| 38.380 | 2.34346 | 80.7558 | 335.759 |
| 38.836 | 2.31698 | 487.645 | 744.980 |
| 39.690 | 2.26910 | 234.687 | 511.553 |

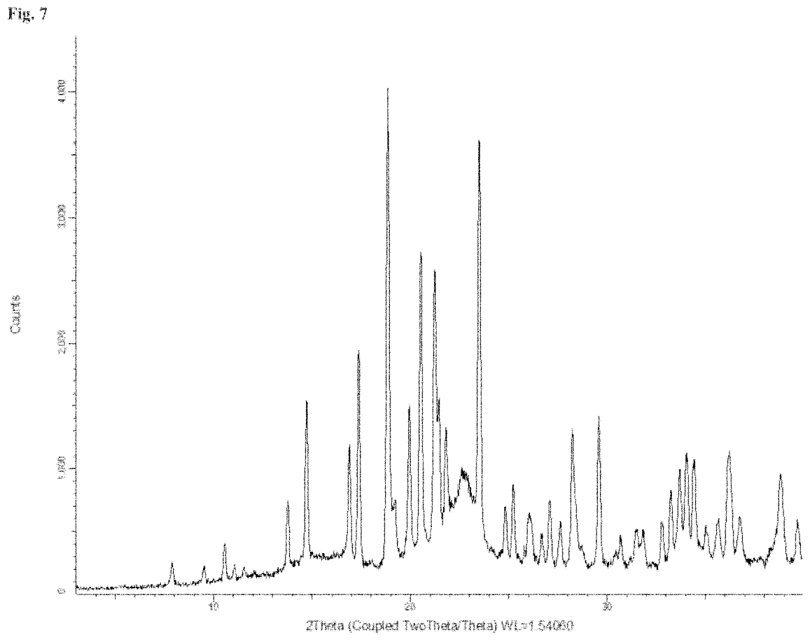

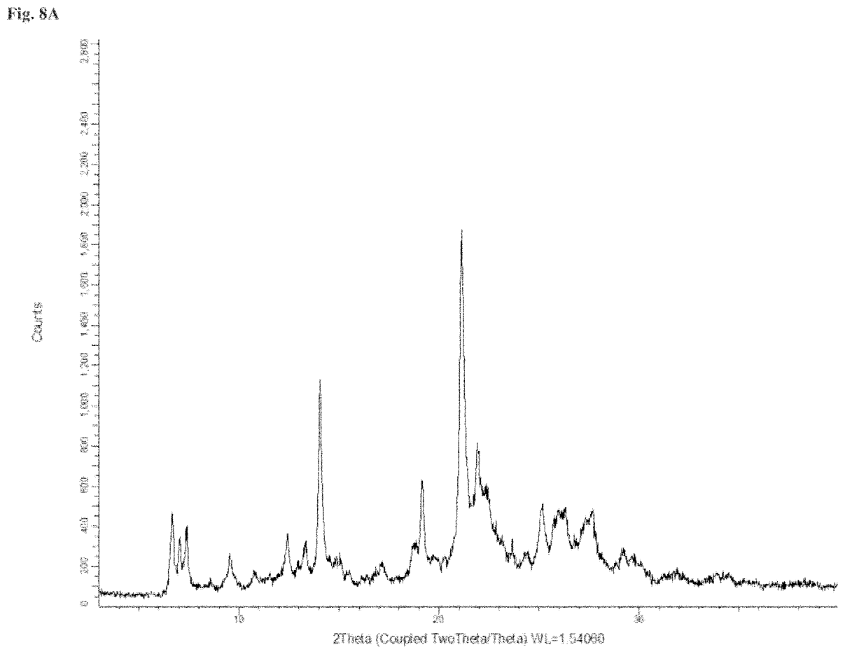

In one specific embodiment, the crystalline Form G of the pamoate salt solvate exhibits an XRPD pattern that is substantially similar to FIG. 7.

### Form H

In one embodiment, the present disclosure relates to a crystalline Form H of the pamoate salt solvate. In one embodiment, Form H of the pamoate salt is an N,N-dimethylacetamide (DMAc) solvate. In one embodiment, Form H is of medium crystallinity. In one embodiment, Form H has an imidazolium:pamoic acid stoichiometry of about 1:1. In one embodiment, Form H comprises DMAc in about 13.0 equiv. as determined by ¹H NMR. In one embodiment, Form H comprises DMAc in about 60.1% by weight.

In one embodiment, Form H of the pamoate salt solvate may comprise of a mixture of one or more forms of the pamoate salt or solvate thereof. In some embodiments, the crystalline form of the pamoate salt or solvate thereof may comprise of substantially pure form of Form H. In one embodiment, the crystalline form of the pamoate salt or solvate thereof may comprise of over about 99.9%, about 99.8%, about 99.7%, about 99.6%, about 99.5%, about 99.4%, about 99.3%, about 99.2%, about 99.1%, or about 99.0% of Form H. In another embodiment, the crystalline form of the pamoate salt or solvate thereof may comprise over about 99%, 98%, 97%, 96%, 95%, 94%, 93%, 92%, 91%, or 90% of Form H. In some embodiments, the crystalline form of the pamoate salt or solvate thereof may comprise over about 90%, 85%, 80%, 75%, 70%, 65%, 60%, 55%, 50%, 45%, or 40% of Form H.

In one embodiment, Form H of the pamoate salt solvate comprises one or both of Form A and Form B of the pamoate salt.

In one embodiment, Form H of the pamoate salt solvate is isolated and purified. In one embodiment, the purified Form H of the pamoate salt solvate is at least 95%, at least 96%, at least 97%, at least 98%, at least 99%, or at least 99.5% pure.

In one embodiment, crystalline Form H of the pamoate salt solvate exhibits an XRPD pattern comprising peaks at about 14.06 and 21.14 degrees two-theta with the margin of error of about ±0.5; about ±0.4; about ±0.3; about ±0.2; about ±0.1; about ±0.05; or less. In another embodiment, the XRPD of the crystalline Form H further comprises peaks at about 19.16, 21.93, and 22.36 degrees two-theta with the margin of error of about ±0.5; about ±0.4; about ±0.3; about ±0.2; about ±0.1; about ±0.05; or less. In further embodiment, the crystalline Form H further comprises at least one peak at about 6.67 or 7.36 degrees two-theta with the margin of error of about ±0.5; about ±0.4; about ±0.3; about ±0.2; about ±0.1; about ±0.05; or less.

In one embodiment, crystalline Form H of the pamoate salt solvate exhibits an XRPD pattern comprising peaks at about 14.06±0.2 and 21.14±0.2 degrees two-theta. In one embodiment, crystalline Form H exhibits an XRPD pattern comprising two of the ten most intense peaks at about 14.06±0.2 and 21.14±0.2 degrees two-theta. In one embodiment, crystalline Form H exhibits an XRPD pattern comprising two of the five most intense peaks at about 14.06±0.2 and 21.14±0.2 degrees two-theta.

In one embodiment, crystalline Form H of the pamoate salt solvate exhibits an XRPD pattern comprising peaks at about 19.16±0.2, 21.93±0.2, and 22.36±0.2 degrees two-theta. In one embodiment, crystalline Form H exhibits an XRPD pattern comprising three of the ten most intense peaks at about 19.16±0.2, 21.93±0.2, and 22.36±0.2 degrees two-theta. In one embodiment, crystalline Form H exhibits an XRPD pattern comprising three of the five most intense peaks at about 19.16±0.2, 21.93±0.2, and 22.36±0.2 degrees two-theta.

In one embodiment, crystalline Form H of the pamoate salt solvate exhibits an XRPD pattern comprising at least two peaks selected from about 14.06±0.2, 19.16±0.2, 21.14±0.2, 21.93±0.2, or 22.36±0.2 degrees two-theta. In one embodiment, crystalline Form H of the pamoate salt solvate exhibits an XRPD pattern comprising at least three peaks selected from about 14.06±0.2, 19.16±0.2, 21.14±0.2, 21.93±0.2, or 22.36±0.2 degrees two-theta. In one embodiment, crystalline Form H of the pamoate salt solvate exhibits an XRPD pattern comprising at least four peaks selected from about 14.06±0.2, 19.16±0.2, 21.14±0.2, 21.93±0.2, or 22.36±0.2 degrees two-theta. In one embodiment, crystalline Form H of the pamoate salt solvate exhibits an XRPD pattern comprising peaks at about 14.06±0.2, 19.16±0.2, 21.14±0.2, 21.93±0.2, and 22.36±0.2 degrees two-theta.

In one embodiment, crystalline Form H of the pamoate salt solvate exhibits an XRPD pattern comprising at least two peaks, at least three peaks, at least four peaks, at least five peaks, or at least six peaks selected from about 6.67±0.2, 7.36±0.2, 14.06±0.2, 19.16±0.2, 21.14±0.2, 21.93±0.2, or 22.36±0.2 degrees two-theta. In one embodiment, crystalline Form H of the pamoate salt solvate exhibits an XRPD pattern comprising peaks at about 6.67±0.2, 7.36±0.2, 14.06±0.2, 19.16±0.2, 21.14±0.2, 21.93±0.2, and 22.36±0.2 degrees two-theta.

In one embodiment, the crystalline Form H of the pamoate salt solvate exhibits an XRPD comprising peaks shown in Table 8 below. In one embodiment, the crystalline Form H of the pamoate salt exhibits an XRPD comprising at least five most intense peaks shown in Table 8 below (net intensity). In one embodiment, the crystalline Form H of the pamoate salt exhibits an XRPD comprising at least six, at least seven, at least eight, at least nine, at least ten most intense peaks shown in Table 8 below (net intensity).

TABLE 8
XRPD Table of Form H of the pamoate salt solvate

| Angle (°2θ) | D Value (Å) | Net Intensity | Gross Intensity |
|---|---|---|---|
| 6.668 | 13.24565 | 243.682 | 312.079 |
| 7.050 | 12.52879 | 178.953 | 255.278 |
| 7.355 | 12.00982 | 206.279 | 288.029 |
| 8.542 | 10.34306 | 30.1229 | 125.321 |
| 9.551 | 9.25311 | 100.491 | 199.478 |
| 10.786 | 8.19609 | 50.3138 | 158.949 |
| 12.431 | 7.11461 | 154.187 | 288.336 |
| 13.018 | 6.79496 | 35.9549 | 173.535 |
| 13.310 | 6.64692 | 113.716 | 251.888 |
| 14.058 | 6.29460 | 667.199 | 803.525 |
| 15.059 | 5.87837 | 95.6971 | 221.974 |
| 15.456 | 5.72841 | 26.1599 | 146.059 |
| 16.865 | 5.25275 | 31.4666 | 157.288 |
| 17.161 | 5.16279 | 48.9050 | 178.302 |
| 18.837 | 4.70705 | 128.621 | 290.068 |
| 19.157 | 4.62924 | 319.250 | 489.864 |
| 21.137 | 4.19990 | 1149.30 | 1357.01 |
| 21.934 | 4.04909 | 420.602 | 633.653 |
| 22.358 | 3.97316 | 262.994 | 476.651 |
| 23.673 | 3.75535 | 111.807 | 317.438 |
| 24.388 | 3.64680 | 32.6201 | 239.889 |
| 25.161 | 3.53655 | 192.694 | 407.197 |
| 25.844 | 3.44464 | 142.079 | 360.153 |
| 26.228 | 3.39503 | 181.553 | 399.860 |
| 27.409 | 3.25137 | 133.679 | 344.705 |
| 27.624 | 3.22659 | 178.115 | 386.518 |
| 29.219 | 3.05397 | 77.6778 | 253.908 |
| 29.666 | 3.00891 | 52.4475 | 215.616 |
| 30.091 | 2.96746 | 62.3567 | 211.545 |
| 33.871 | 2.64442 | 35.0118 | 151.426 |

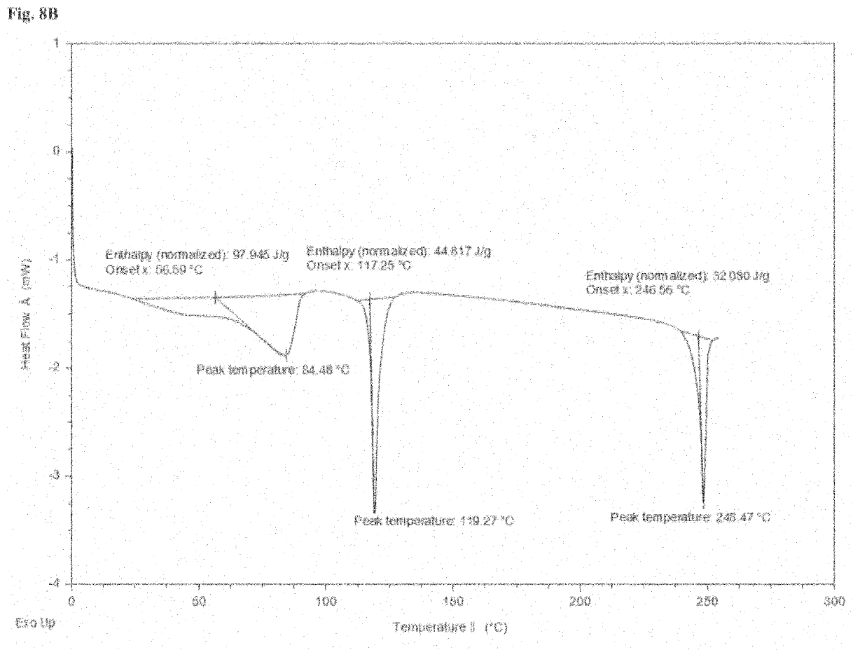

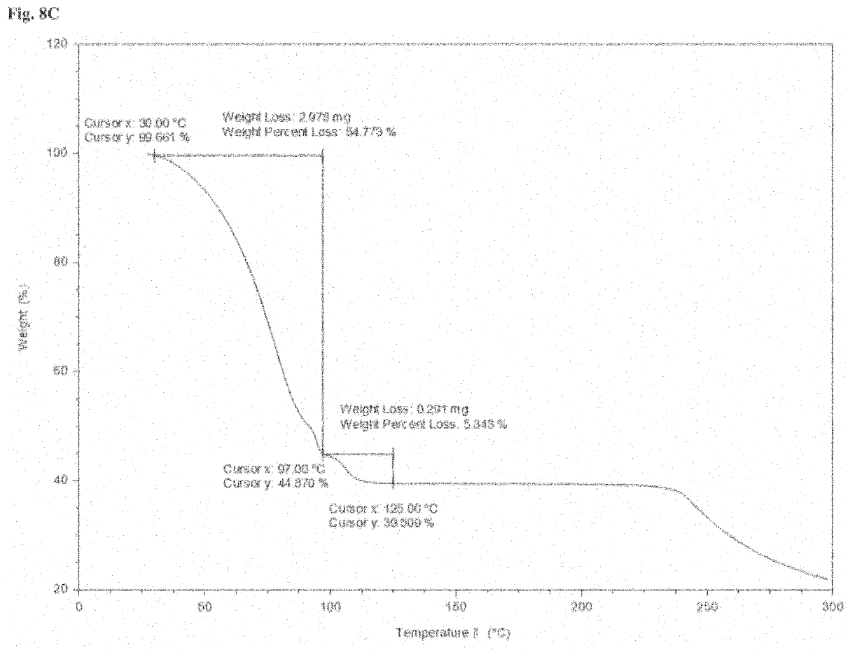

In one specific embodiment, the crystalline Form H of the pamoate salt solvate exhibits an XRPD pattern that is substantially similar to FIG. 8A.

In one embodiment, the crystalline Form H of the pamoate salt solvate exhibits a DSC thermogram comprising a desolvation peak at about 57° C. (onset) with the error of margin of about ±2.5; about ±2.0; about ±1.5; about ±1.0; about ±0.5; or less. In one embodiment, the enthalpy of desolvation is about 98 J/g for the peak at about 57° C. (onset) by DSC. In one embodiment, the crystalline Form H exhibits a DSC thermogram comprising a desolvation peak at about 117° C. (onset) with the error of margin of about ±2.5; about ±2.0; about ±1.5; about ±1.0; about ±0.5; or less (peak maximum at about 119° C.). In one embodiment, the enthalpy of desolvation is about 45 J/g for the peak at about 117° C. (onset) by DSC. In one embodiment, the crystalline Form H exhibits a DSC thermogram comprising an endotherm peak at about 247° C. (onset) with the error of margin of about ±2.5; about ±2.0; about ±1.5; about ±1.0; about ±0.5; or less. In one embodiment, the crystalline Form H exhibits a DSC thermogram comprising an endotherm peak at about 249° C. (peak maximum) with the error of margin of about ±2.5; about ±2.0; about ±1.5; about ±1.0; about ±0.5; or less. In one embodiment, the crystalline Form H of the pamoate salt exhibits a DSC thermogram that is substantially similar to FIG. 8B.

In one embodiment, the crystalline Form H of the pamoate salt solvate exhibits a TGA thermogram substantially similar to FIG. 8C. In one embodiment, crystalline Form H shows about 54.8% weight loss in the range of about 35° C. to about 97° C. by TGA. In one embodiment, crystalline Form H shows about 5.3% weight loss in the range of about 97° C. to about 125° C. by TGA.

### Form I

In one embodiment, the present disclosure relates to a crystalline Form I of the pamoate salt solvate. In one embodiment, Form I of the pamoate salt is a hexafluoroisopropyl acrylate (HFIPA) solvate. In one embodiment, Form I is of low crystallinity. In one embodiment, Form I has an imidazolium:pamoic acid stoichiometry of about 1:3.5. In one embodiment, Form I comprises HFIPA in about 1.0 equiv. as determined by ¹H NMR. In one embodiment, Form I comprises HFIPA in about 8.9% by weight.

In one embodiment, Form I of the pamoate salt solvate may comprise of a mixture of one or more forms of the pamoate salt or solvate thereof. In some embodiments, the crystalline form of the pamoate salt or solvate thereof may comprise of substantially pure form of Form I. In one embodiment, the crystalline form of the pamoate salt or solvate thereof may comprise of over about 99.9%, about 99.8%, about 99.7%, about 99.6%, about 99.5%, about 99.4%, about 99.3%, about 99.2%, about 99.1%, or about 99.0% of Form I. In another embodiment, the crystalline form of the pamoate salt or solvate thereof may comprise over about 99%, 98%, 97%, 96%, 95%, 94%, 93%, 92%, 91%, or 90% of Form I. In some embodiments, the crystalline form of the pamoate salt or solvate thereof may comprise over about 90%, 85%, 80%, 75%, 70%, 65%, 60%, 55%, 50%, 45%, or 40% of Form I.

In one embodiment, Form I of the pamoate salt solvate comprises one or both of Form A and Form B of the pamoate salt.

In one embodiment, Form I of the pamoate salt solvate is isolated and purified. In one embodiment, the purified Form I of the pamoate salt solvate is at least 95%, at least 96%, at least 97%, at least 98%, at least 99%, or at least 99.5% pure.

In one embodiment, crystalline Form I of the pamoate salt solvate exhibits an XRPD pattern comprising peaks at about 4.73 and 9.45 degrees two-theta with the margin of error of about ±0.5; about ±0.4; about ±0.3; about ±0.2; about ±0.1; about ±0.05; or less.

In one embodiment, crystalline Form I of the pamoate salt solvate exhibits an XRPD pattern comprising peaks at about 4.73±0.2 and 9.45±0.2 degrees two-theta. In one embodiment, crystalline Form I of the pamoate salt solvate exhibits an XRPD pattern comprising two most intense peaks at about 4.73±0.2 and 9.45±0.2 degrees two-theta. In one embodiment, crystalline Form I of the pamoate salt solvate exhibits an XRPD pattern comprising only two peaks at about 4.73±0.2 and 9.45±0.2 degrees two-theta.

In one embodiment, the crystalline Form I of the pamoate salt solvate exhibits an XRPD comprising peaks shown in Table 9 below.

TABLE 9
XRPD Table of Form I of the pamoate salt solvate

| Angle (°2θ) | D Value (Å) | Net Intensity | Gross Intensity |
|---|---|---|---|
| 4.729 | 18.67040 | 714.975 | 758.710 |
| 9.449 | 9.35204 | 196.250 | 257.289 |

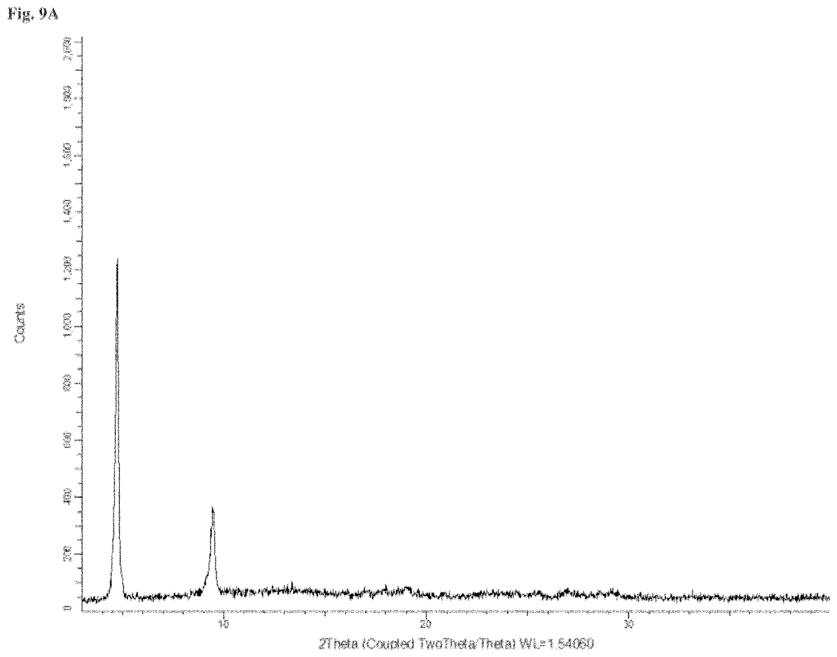

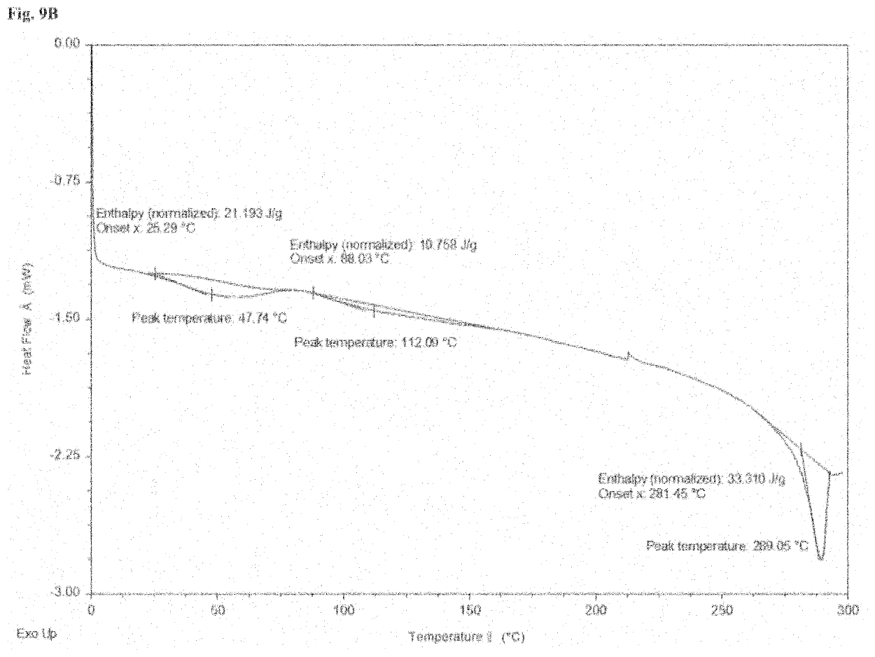

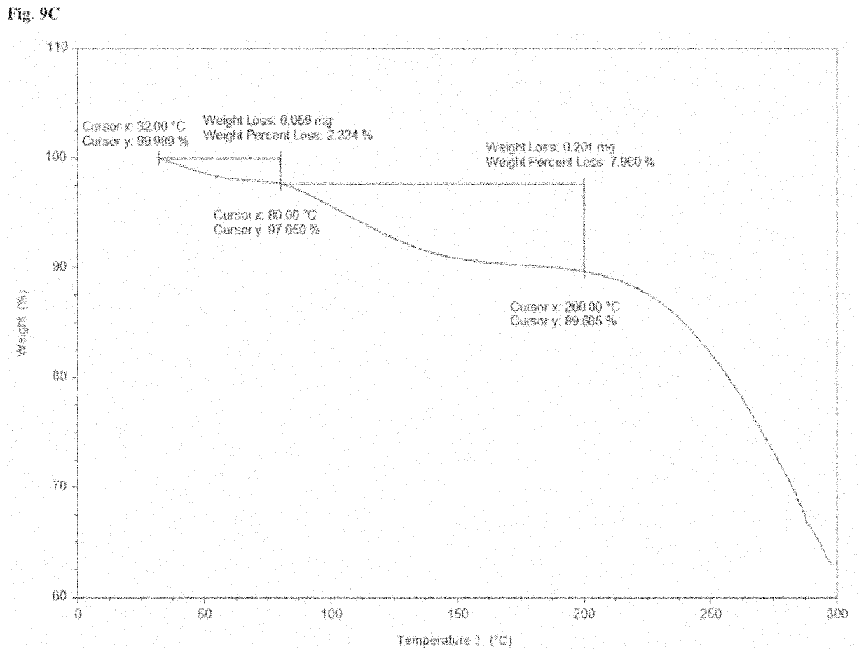

In one specific embodiment, the crystalline Form I of the pamoate salt solvate exhibits an XRPD pattern that is substantially similar to FIG. 9A.

In one embodiment, the crystalline Form I of the pamoate salt solvate exhibits a DSC thermogram comprising a desolvation peak at about 25° C. (onset) with the error of margin of about ±2.5; about ±2.0; about ±1.5; about ±1.0; about ±0.5; or less. In one embodiment, the enthalpy of desolvation is about 21 J/g for the peak at about 25° C. (onset) by DSC. In one embodiment, the crystalline Form I exhibits a DSC thermogram comprising a desolvation peak at about 88° C. (onset) with the error of margin of about ±2.5; about ±2.0; about ±1.5; about 1.0; about ±0.5; or less. In one embodiment, the enthalpy of desolvation is about 11 J/g for the peak at about 88° C. (onset) by DSC. In one embodiment, the crystalline Form I exhibits a DSC thermogram comprising an endotherm peak at about 282° C. (onset) with the error of margin of about ±2.5; about ±2.0; about ±1.5; about 1.0; about ±0.5; or less. In one embodiment, the crystalline Form I exhibits a DSC thermogram comprising an endotherm peak at about 289° C. (peak maximum) with the error of margin of about ±2.5; about ±2.0; about ±1.5; about ±1.0; about ±0.5; or less. In one embodiment, the crystalline Form I of the pamoate salt exhibits a DSC thermogram that is substantially similar to FIG. 9B.

In one embodiment, the crystalline Form I of the pamoate salt solvate exhibits a TGA thermogram substantially similar to FIG. 9C. In one embodiment, crystalline Form I shows about 2.3% weight loss in the range of about 35° C. to about 80° C. by TGA. In one embodiment, crystalline Form I shows about 8.0% weight loss in the range of about 80° C. to about 200° C. by TGA.

### Form J

In one embodiment, the present disclosure relates to a crystalline Form J of the pamoate salt solvate. In one embodiment, Form J of the pamoate salt is an N,N-dimethylformamide (DMF) solvate. In one embodiment, Form J is of high crystallinity. In one embodiment, Form J has an imidazolium:pamoic acid stoichiometry of about 1:1. In one embodiment, Form J comprises DMF in about 6.3 equiv. as determined by ¹H NMR. In one embodiment, Form J comprises DMF in about 38.0% by weight.

In one embodiment, Form J of the pamoate salt solvate may comprise of a mixture of one or more forms of the pamoate salt or solvate thereof. In some embodiments, the crystalline form of the pamoate salt or solvate thereof may comprise of substantially pure form of Form J. In one embodiment, the crystalline form of the pamoate salt or solvate thereof may comprise of over about 99.9%, about 99.8%, about 99.7%, about 99.6%, about 99.5%, about 99.4%, about 99.3%, about 99.2%, about 99.1%, or about 99.0% of Form J. In another embodiment, the crystalline form of the pamoate salt or solvate thereof may comprise over about 99%, 98%, 97%, 96%, 95%, 94%, 93%, 92%, 91%, or 90% of Form J. In some embodiments, the crystalline form of the pamoate salt or solvate thereof may comprise over about 90%, 85%, 80%, 75%, 70%, 65%, 60%, 55%, 50%, 45%, or 40% of Form J.

In one embodiment, Form J of the pamoate salt solvate comprises one or both of Form A and Form B of the pamoate salt.

In one embodiment, Form J of the pamoate salt solvate is isolated and purified. In one embodiment, the purified Form J of the pamoate salt solvate is at least 95%, at least 96%, at least 97%, at least 98%, at least 99%, or at least 99.5% pure.

In one embodiment, crystalline Form J of the pamoate salt solvate exhibits an XRPD pattern comprising peaks at about 5.82, 11.81, and 11.62 degrees two-theta with the margin of error of about ±0.5; about ±0.4; about ±0.3; about ±0.2; about 0.1; about ±0.05; or less. In another embodiment, the XRPD of the crystalline Form J further comprises peaks at about 17.46, 23.49, and 23.61 degrees two-theta with the margin of error of about ±0.5; about ±0.4; about ±0.3; about ±0.2; about ±0.1; about ±0.05; or less. In further embodiment, the crystalline Form J further comprises peaks at about 19.51 and 21.26 degrees two-theta with the margin of error of about ±0.5; about ±0.4; about ±0.3; about 0.2; about 0.1; about ±0.05; or less.

In one embodiment, crystalline Form J of the pamoate salt solvate exhibits an XRPD pattern comprising peaks at about 5.82±0.2, 11.81±0.2, and 11.62±0.2 degrees two-theta. In one embodiment, crystalline Form J exhibits an XRPD pattern comprising three of the ten most intense peaks at about 5.82±0.2, 11.81±0.2, and 11.62±0.2 degrees two-theta. In one embodiment, crystalline Form J exhibits an XRPD pattern comprising three of the six most intense peaks at about 5.82±0.2, 11.81±0.2, and 11.62±0.2 degrees two-theta.

In one embodiment, crystalline Form J of the pamoate salt solvate exhibits an XRPD pattern comprising peaks at about 17.46±0.2, 23.49±0.2, and 23.61±0.2 degrees two-theta. In one embodiment, crystalline Form J exhibits an XRPD pattern comprising three of the ten most intense peaks at about 17.46±0.2, 23.49±0.2, and 23.61±0.2 degrees two-theta. In one embodiment, crystalline Form J exhibits an XRPD pattern comprising three of the six most intense peaks at about 17.46±0.2, 23.49±0.2, and 23.61±0.2 degrees two-theta.

In one embodiment, crystalline Form J of the pamoate salt solvate exhibits an XRPD pattern comprising at least two peaks selected from about 5.82±0.2, 11.81±0.2, 11.62±0.2, 17.46±0.2, 23.49±0.2, or 23.61±0.2 degrees two-theta. In one embodiment, crystalline Form J of the pamoate salt solvate exhibits an XRPD pattern comprising at least three peaks selected from about 5.82±0.2, 11.81±0.2, 11.62±0.2, 17.46±0.2, 23.49±0.2, or 23.61±0.2 degrees two-theta. In one embodiment, crystalline Form J of the pamoate salt solvate exhibits an XRPD pattern comprising at least four peaks selected from about 5.82±0.2, 11.81±0.2, 11.62±0.2, 17.46±0.2, 23.49±0.2, or 23.61±0.2 degrees two-theta. In one embodiment, crystalline Form J of the pamoate salt solvate exhibits an XRPD pattern comprising at least five peaks selected from about 5.82±0.2, 11.81±0.2, 11.62±0.2, 17.46±0.2, 23.49±0.2, or 23.61±0.2 degrees two-theta. In one embodiment, crystalline Form J of the pamoate salt solvate exhibits an XRPD pattern comprising peaks at about 5.82±0.2, 11.81±0.2, 11.62±0.2, 17.46±0.2, 23.49±0.2, and 23.61±0.2 degrees two-theta.

In one embodiment, crystalline Form J of the pamoate salt solvate exhibits an XRPD pattern comprising at least two peaks, at least three peaks, at least four peaks, at least five peaks, at least six peaks, at least seven peaks, at least eight peaks, or at least nine peaks selected from about 5.82±0.2, 11.09±0.2, 11.81±0.2, 11.62±0.2, 17.46±0.2, 19.51±0.2, 21.26±0.2, 23.49±0.2, 23.61±0.2 or 28.32±0.2 degrees two-theta. In one embodiment, crystalline Form J of the pamoate salt solvate exhibits an XRPD pattern comprising peaks at about 5.82±0.2, 11.09±0.2, 11.81±0.2, 11.62±0.2, 17.46±0.2, 19.51±0.2, 21.26±0.2, 23.49±0.2, 23.61±0.2 and 28.32±0.2 degrees two-theta.

In one embodiment, the crystalline Form J of the pamoate salt solvate exhibits an XRPD comprising peaks shown in Table 10 below. In one embodiment, the crystalline Form J of the pamoate salt exhibits an XRPD comprising at least five most intense peaks shown in Table 10 below (net intensity). In one embodiment, the crystalline Form J of the pamoate salt exhibits an XRPD comprising at least six, at least seven, at least eight, at least nine, at least ten most intense peaks shown in Table 10 below (net intensity).

TABLE 10
XRPD Table of Form J of the pamoate salt solvate

| | Angle (°2θ) | D Value (Å) | Net Intensity | Gross Intensity |
|---|---|---|---|---|
| 1 | 5.820 | 15.17244 | 1313.33 | 1365.76 |
| 2 | 7.920 | 11.15461 | 18.4191 | 81.7818 |
| 3 | 8.337 | 10.59676 | 354.972 | 422.605 |
| 4 | 8.457 | 10.44686 | 139.729 | 208.210 |
| 5 | 9.191 | 9.61435 | 151.511 | 221.534 |
| 6 | 9.993 | 8.84435 | 73.9501 | 146.625 |
| 7 | 11.086 | 7.97455 | 432.962 | 514.107 |
| 8 | 11.248 | 7.86025 | 284.877 | 367.949 |
| 9 | 11.624 | 7.60711 | 1897.71 | 1984.09 |
| 10 | 11.813 | 7.48546 | 2387.48 | 2474.91 |
| 11 | 14.012 | 6.31521 | 72.2489 | 148.042 |
| 12 | 14.580 | 6.07072 | 28.6214 | 105.207 |
| 13 | 15.164 | 5.83800 | 95.2094 | 174.270 |
| 14 | 15.481 | 5.71908 | 63.2207 | 143.784 |
| 15 | 15.832 | 5.59318 | 141.591 | 223.805 |
| 16 | 16.169 | 5.47745 | 21.6395 | 104.087 |
| 17 | 16.704 | 5.30304 | 299.892 | 387.445 |
| 18 | 17.457 | 5.07601 | 840.536 | 937.062 |
| 19 | 17.704 | 5.00569 | 224.579 | 322.608 |
| 20 | 18.586 | 4.77007 | 190.448 | 288.024 |
| 21 | 19.505 | 4.54739 | 705.241 | 804.612 |
| 22 | 19.793 | 4.48186 | 97.4143 | 198.653 |
| 23 | 20.052 | 4.42450 | 40.5792 | 142.675 |
| 24 | 20.486 | 4.33185 | 346.270 | 448.045 |
| 25 | 21.258 | 4.17628 | 720.070 | 822.725 |
| 26 | 21.626 | 4.10599 | 78.2940 | 180.656 |
| 27 | 22.244 | 3.99322 | 84.0298 | 192.147 |
| 28 | 23.103 | 3.84667 | 180.239 | 295.875 |
| 29 | 23.485 | 3.78508 | 1098.71 | 1214.92 |
| 30 | 23.608 | 3.76563 | 1115.26 | 1231.29 |
| 31 | 24.201 | 3.67455 | 98.9925 | 211.696 |
| 32 | 25.129 | 3.54097 | 54.2764 | 153.524 |
| 33 | 25.511 | 3.48886 | 54.1754 | 152.875 |
| 34 | 26.544 | 3.35537 | 165.917 | 275.617 |
| 35 | 27.104 | 3.28729 | 41.5069 | 155.010 |
| 36 | 27.851 | 3.20073 | 219.488 | 333.501 |
| 37 | 28.319 | 3.14890 | 518.745 | 630.539 |
| 38 | 30.580 | 2.92104 | 139.081 | 227.596 |
| 39 | 31.148 | 2.86908 | 117.823 | 208.532 |
| 40 | 31.942 | 2.79959 | 143.663 | 234.613 |
| 41 | 33.505 | 2.67245 | 27.7773 | 113.512 |
| 42 | 34.353 | 2.60837 | 36.3002 | 123.082 |
| 43 | 34.839 | 2.57314 | 28.1661 | 113.260 |

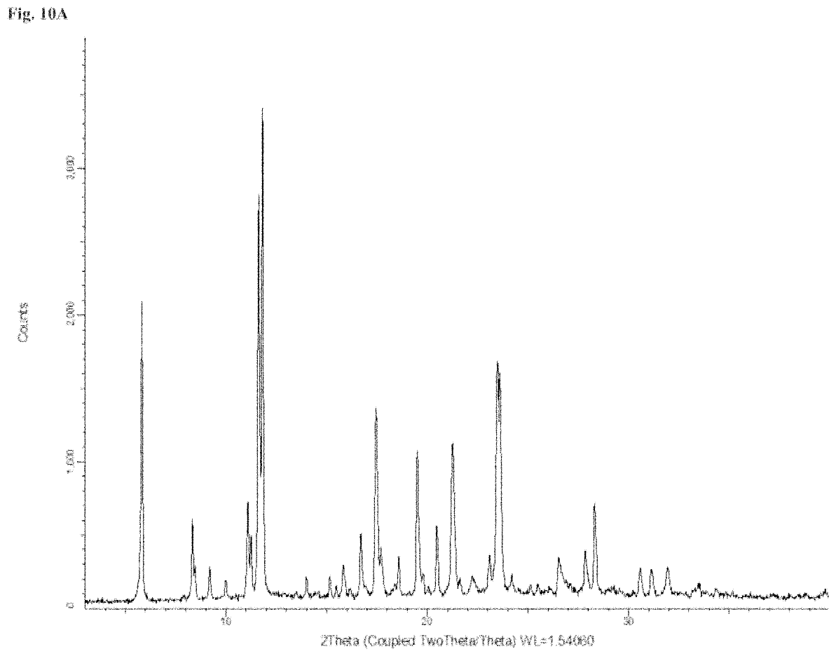

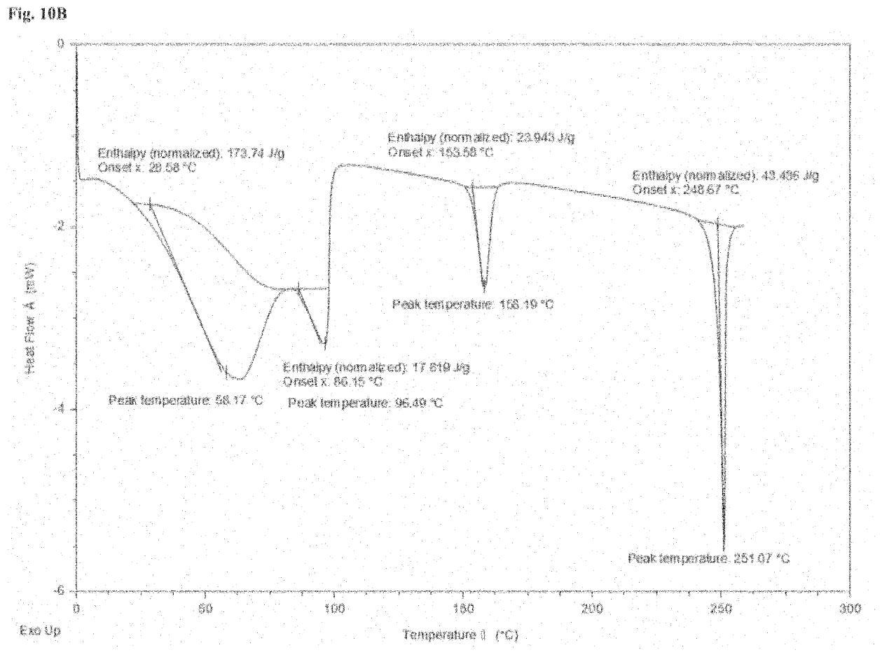

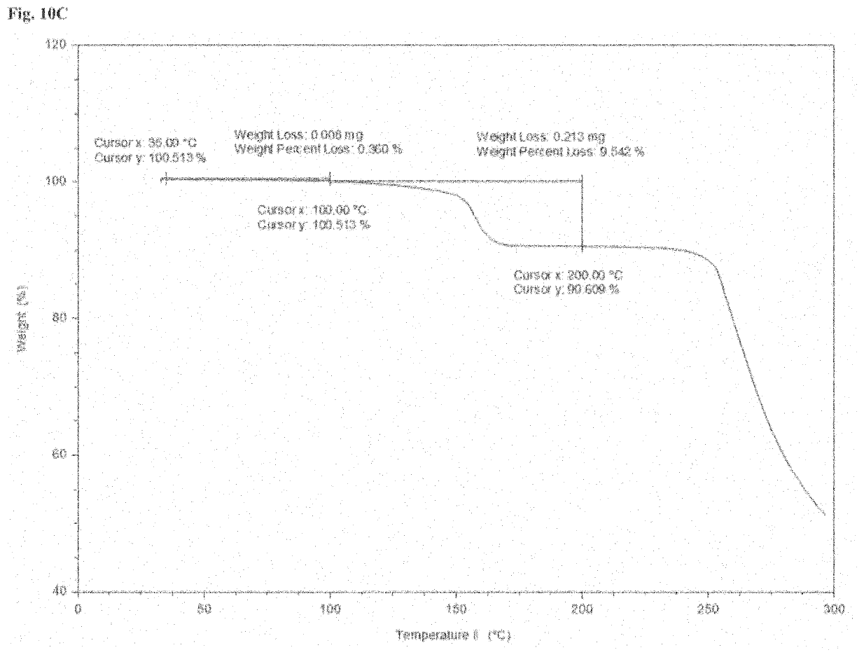

In one specific embodiment, the crystalline Form J of the pamoate salt solvate exhibits an XRPD pattern that is substantially similar to FIG. 10A.

In one embodiment, the crystalline Form J of the pamoate salt solvate exhibits a DSC thermogram comprising a desolvation peak at about 29° C. (onset) with the error of margin of about ±2.5; about ±2.0; about ±1.5; about ±1.0; about ±0.5; or less. In one embodiment, the enthalpy of desolvation is about 174 J/g for the peak at about 29° C. (onset) by DSC. In one embodiment, the crystalline Form J exhibits a DSC thermogram comprising a desolvation peak at about 86° C. (onset) with the error of margin of about ±2.5; about ±2.0; about ±1.5; about ±1.0; about ±0.5; or less. In one embodiment, the enthalpy of desolvation is about 18 J/g for the peak at about 86° C. (onset) by DSC. In one embodiment, the crystalline Form J exhibits a DSC thermogram comprising a desolvation peak at about 154° C. (onset) with the error of margin of about ±2.5; about ±2.0; about ±1.5; about ±1.0; about ±0.5; or less. In one embodiment, the enthalpy of desolvation is about 24 J/g for the peak at about 154° C. (onset) by DSC. In one embodiment, the crystalline Form J exhibits a DSC thermogram comprising an endotherm peak at about 249° C. (onset) with the error of margin of about ±2.5; about ±2.0; about ±1.5; about ±1.0; about ±0.5; or less. In one embodiment, the crystalline Form J exhibits a DSC thermogram comprising an endotherm peak at about 251° C. (peak maximum) with the error of margin of about ±2.5; about ±2.0; about ±1.5; about ±1.0; about ±0.5; or less. In one embodiment, the crystalline Form J of the pamoate salt exhibits a DSC thermogram that is substantially similar to FIG. 10B.

In one embodiment, the crystalline Form J of the pamoate salt solvate exhibits a TGA thermogram substantially similar to FIG. 10C. In one embodiment, crystalline Form J shows about 0.4% weight loss in the range of about 35° C. to about 100° C. by TGA. In one embodiment, crystalline Form J shows about 9.5% weight loss in the range of about 100° C. to about 200° C. by TGA.

### Amorphous Form

In one embodiment, the present disclosure relates to solid forms of the pamoate salt or solvate thereof. In one embodiment, the present disclosure relates to an amorphous form of the pamoate salt or solvate thereof. In one embodiment, the present disclosure relates to an amorphous form of anhydrous or non-solvated pamoate salt. In one embodiment, the present disclosure relates to an amorphous form of the pamoate salt solvate.

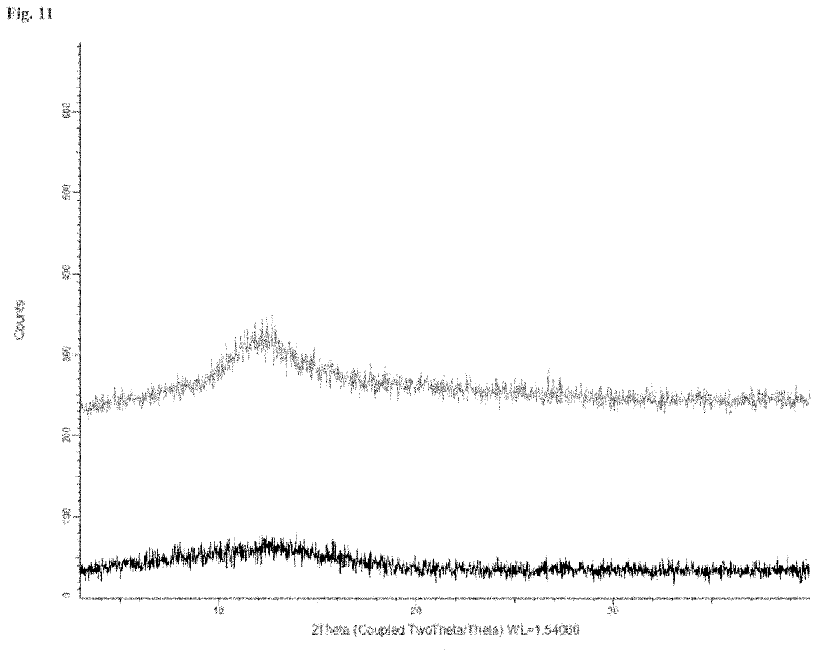

In one embodiment, the amorphous form of the pamoate salt or solvate thereof exhibits an XRPD pattern that is substantially similar to either spectrum of FIG. 11. In one embodiment, the amorphous form of the pamoate salt or solvate thereof exhibits an XRPD pattern that is substantially similar FIG. 11 (top spectrum). In one embodiment, the amorphous form of the pamoate salt or solvate thereof exhibits an XRPD pattern that is substantially similar FIG. 11 (bottom spectrum).

### Pharmaceutical Compositions and Formulations

The present invention also provides a pharmaceutical composition comprising the pamoate salt or solvate thereof and a pharmaceutically acceptable excipient or carrier. In some embodiments, the pharmaceutical composition comprises an effective amount of the pamoate salt or solvate thereof. In some embodiments, the pamoate salt or solvate thereof is crystalline or amorphous. In some embodiments, the pamoate salt or solvate thereof is one or more of Forms A-J or an amorphous form. In some embodiments, the pharmaceutical composition comprises Form B of the pamoate salt. The excipients are added to the formulation for a variety of purposes.

The present invention also provides a pharmaceutical composition comprising one or more forms of the pamoate salt or solvate thereof. In one embodiment, the pharmaceutical composition comprises the pamoate salt or solvate salt thereof as a mixture of different forms. In one embodiment, the pharmaceutical composition comprises a mixture of a crystalline form and an amorphous form of the pamoate salt or solvate thereof. In some embodiments of the pharmaceutical composition, the combination of one or more forms of the pamoate salt or solvate thereof provides an effective amount of the pamoate salt or solvate thereof. In some embodiments, the pharmaceutical composition comprises Form B of the pamoate salt and one or more forms of the pamoate salt or solvate selected from Form A, Forms C-J, or an amorphous form. In some embodiments, Form B of the pamoate salt is present in greater than about 10%, about 20%, about 30%, about 40%, about 50%, about 60%, about 70%, about 80%, about 85%, about 90%, or about 95% of the total amount of the pamoate salt or solvate in the pharmaceutical composition. In some embodiments, Form B of the pamoate salt is present in greater than about 90%, about 910%, about 92%, about 93%, about 94%, about 95%, about 96%, about 97%, about 98%, about 99%, or about 99.5% of the total amount of the pamoate salt or solvate in the pharmaceutical composition. In some embodiments of the pharmaceutical composition comprising Form B of the pamoate salt, any one of Form A, Forms C-J, or an amorphous form of the pamoate salt or solvate thereof is present in about 0.1%, about 0.2%, about 0.3%, about 0.5%, about 0.6%, about 0.7%, about 0.8%, about 0.9%, about 1%, about 2%, about 3%, about 4%, about 5%, about 6%, about 7%, about 8%, about 9%, about 10%, about 15%, about 20%, about 25%, or about 30% of the total amount of the pamoate salt or solvate in the pharmaceutical composition.

In one embodiment, the pharmaceutical composition comprises Form B of the pamoate salt in about 99.9%, about 99.8%, about 99.7%, about 99.6%, about 99.5%, about 99.4%, about 99.3%, about 99.2%, about 99.1%, or about 99.0% of the total amount of the pamoate salt or solvate in the pharmaceutical composition. In one embodiment, the pharmaceutical composition comprises Form B of the pamoate salt in about 99%, 98%, 97%, 96%, 95%, 94%, 93%, 92%, 91%, or 90% of the total amount of the pamoate salt or solvate in the pharmaceutical composition. In one embodiment, the pharmaceutical composition comprises Form B of the pamoate salt in about 90%, 85%, 80%, 75%, 70%, 65%, 60%, 55%, 50%, 45%, 40%, 35%, 30%, 25%, or 20% of the total amount of the pamoate salt or solvate in the pharmaceutical composition. In one embodiment, the pharmaceutical composition comprises Form B of the pamoate salt in about 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 11%, 12%, 13%, 14%, 15%, 16%, 17%, 18%, 18%, or 20% of the total amount of the pamoate salt or solvate in the pharmaceutical composition.

In one embodiment, the pharmaceutical composition comprises a crystalline form of the pamoate salt or solvate thereof in about 99.9%, about 99.8%, about 99.7%, about 99.6%, about 99.5%, about 99.4%, about 99.3%, about 99.2%, about 99.1%, or about 99.0% of the total amount of the pamoate salt or solvate in the pharmaceutical composition. In one embodiment, the pharmaceutical composition comprises a crystalline form of the pamoate salt in about 99%, 98%, 97%, 96%, 95%, 94%, 93%, 92%, 91%, or 90% of the total amount of the pamoate salt or solvate in the pharmaceutical composition. In one embodiment, the pharmaceutical composition comprises a crystalline form of the pamoate salt in about 90%, 85%, 80%, 75%, 70%, 65%, 60%, 55%, 50%, 45%, 40%, 35%, 30%, 25%, or 20% of the total amount of the pamoate salt or solvate in the pharmaceutical composition. In one embodiment, the pharmaceutical composition comprises a crystalline form of the pamoate salt in about 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10, 1%, 12%, 13%, 14%, 15%, 16%, 17%, 18%, 18%, or 20% of the total amount of the pamoate salt or solvate in the pharmaceutical composition.

In one embodiment of the pharmaceutical composition, the pamoate salt is an anhydrate.

In one embodiment of the pharmaceutical composition, the pharmaceutical composition further comprises one or more additional therapeutically active agent. In one embodiment, the one or more additional therapeutically active agent is a cytotoxic agent or an anticancer agent. In some embodiments, the one or more additional therapeutically active agent is rituximab, docetaxel, carboplatin, paclitaxel, or prednisone. See, RE45105 and U.S. Pat. No. 7,618,992, the disclosures of each are hereby incorporated by reference in their entireties for all purposes.

In some embodiments, the pharmaceutical composition is formulated for administration orally, nasally, transdermally, topically, pulmonary, inhalationally, buccally, sublingually, intraperintoneally, subcutaneously, intramuscularly, intravenously, rectally, intrapleurally, intrathecally, intraportally, or parenterally, or as an implant.

In some embodiments, the pharmaceutical composition comprises about 1 mg to about 1000 mg of the pamoate salt or solvate thereof, or any value or subranges therebetween. In some embodiments, the pharmaceutical composition comprises about 10 mg to about 500 mg of the pamoate salt or solvate thereof, or any value or subranges therebetween. In some embodiments, the pharmaceutical composition comprises about 20 mg to about 250 mg of the pamoate salt or solvate thereof, or any value or subranges therebetween. In some embodiments, the pharmaceutical composition comprises about 25 mg to about 150 mg of the pamoate salt or solvate thereof, or any value or subranges therebetween. In some embodiments, the pharmaceutical composition comprises about 25 mg to about 100 mg of the pamoate salt or solvate thereof, or any value or subranges therebetween.

### Therapeutic Methods

The present disclosure also provides a method of treating cancer comprising administering to a subject in need thereof, an effective amount of any one of pamoate salt or solvate thereof or a pharmaceutical composition thereof as described herein.

In some embodiments, the cancer has a c-Myc rearrangement, amplification, or overexpression, and/or MYCN amplification or overexpression. In some embodiments, the cancer has a c-Myc translocation. In some embodiments, the translocation is to an immunoglobin locus. In some embodiments, the translocation is to an immunoglobin heavy chain locus or to an immunoglobin light chain locus. In some embodiments, c-Myc translocation is t(8;14) (q24; q32), t(2; 8) (p12; q24) and/or t(8; 22) (q24; q11).

In some embodiments, the cancer is a c-Myc overexpressing cancer. In one embodiment, the c-Myc overexpressing cancer is a hematologic cancer or a solid tumor.

In some embodiments, the cancer harbors a c-Myc rearrangement.

In some embodiments, the cancer is a c-Myc driven cancer.

In some embodiments, the cancer is a solid tumor or a hematologic malignancy. In some embodiments, the hematologic malignancy is a myeloma, leukemia, or lymphoma. In some embodiments, the hematologic malignancy is myeloid disorder, lymphoid disorder, leukemia, lymphoma, myelodysplastic syndrome (MDS), myeloproliferative disease (MPD), mast cell disorder, or myeloma. In some embodiments, the hematologic malignancy is acute lymphoblastic leukemia (ALL), T-cell ALL (T-ALL), B-cell ALL (B-ALL), acute myeloid leukemia (AML), granulocytic leukemia, monocytic leukemia, lymphocytic leukemia, chronic lymphocytic leukemia (CLL), chronic myelogenous leukemia (CML), blast phase CML, small lymphocytic lymphoma (SLL), CLL/SLL, Hodgkin lymphoma (HL), non-Hodgkin lymphoma (NHL), B-cell NHL, CD20-positive B cell NHL, T-cell NHL, indolent NHL (iNHL), high-grade B-cell lymphoma (HGBCL), diffuse large B-cell lymphoma (DLBCL), mantle cell lymphoma (MCL), aggressive B-cell NHL, B-cell lymphoma (BCL), Richter's syndrome (RS), T-cell lymphoma (TCL), peripheral T-cell lymphoma (PTCL), cutaneous T-cell lymphoma (CTCL), transformed mycosis fungoides, Sezary syndrome, anaplastic large-cell lymphoma (ALCL), follicular lymphoma (FL), Waldenstrom macroglobulinemia (WM), lymphoplasmacytic lymphoma, Burkitt lymphoma, malignant lymphoma, plasmocytoma, reticulum cell sarcoma, multiple myeloma (MM), metastatic myeloma, amyloidosis, MPD, essential thrombocytosis (ET), myelofibrosis (MF), polycythemia vera (PV), chronic myelomonocytic leukemia (CMML), myelodysplastic syndrome (MDS), high-risk MDS, or low-risk MDS. In some embodiments, the hematologic malignancy is a high-grade B-cell lymphoma (HGBCL) or a diffuse large B-cell lymphoma (DLBCL).

In some embodiments, the cancer is prostate cancer, breast cancer, lung cancer, brain cancer, liver cancer, uterine cancer, pancreatic cancer, gallbladder cancer, bile duct cancer, colon or rectum cancer, urinary bladder cancer, kidney cancer, ovarian cancer, esophageal cancer, gastric (stomach) cancer, head and neck cancer, bone cancer, thyroid cancer, cervical cancer, lymphoma, or leukemia. In some embodiments, the cancer is small cell lung cancer, non-small cell lung cancer (NSCLC), neuroendocrine prostate cancer (NEPC), hormone refractory prostate cancer (HRPC), neuroblastoma, triple negative breast cancer (TNBC), HER2 negative metastatic breast cancer, or hemangiosarcoma.

In some embodiments, the cancer is relapsed or refractory.

The present disclosure also provides a method of inhibiting or reducing the expression of c-Myc protein level in a cancer cell of a subject, comprising administering to the subject, any one of the pamoate salt or solvate thereof or a pharmaceutical composition thereof as described herein.

The present disclosure also provides a method of degrading c-Myc protein in a cancer cell of a subject, comprising administering to the subject, any one of the pamoate salt or solvate thereof or a pharmaceutical composition thereof as described herein.

The present disclosure also provides a method of increasing the expression of cleaved Caspase-3 protein in a subject, comprising administering to the subject, any one of the pamoate salt or solvate thereof or a pharmaceutical composition thereof as described herein.

The present disclosure also provides a method of decreasing the expression of full length Caspase-3 protein in a subject, comprising administering to the subject, any one of the pamoate salt or solvate thereof or a pharmaceutical composition thereof as described herein.

The present disclosure also provides a method of inducing tumor cell death, comprising administering to the subject with cancer or tumor, any one of the pamoate salt or solvate thereof or a pharmaceutical composition thereof as described herein.

The present disclosure also provides a method of inducing apoptosis of cancer cells, comprising administering to the subject with cancer or tumor, any one of the pamoate salt or solvate thereof or a pharmaceutical composition thereof as described herein.

In some embodiments of any one of the methods as disclosed herein, the method comprises further administering one or more additional therapeutically active agent. In some embodiments, the one or more additional therapeutically active agent is a cytotoxic agent or an anticancer agent. In some embodiments, the one or more additional therapeutically active agent is rituximab, docetaxel, carboplatin, paclitaxel, or prednisone.

In some embodiments of any one of the methods as disclosed herein, the pamoate salt or solvate thereof is crystalline and/or amorphous. In some embodiments, the pamoate salt or solvate thereof is any one of Forms A-J and/or an amorphous form. In some embodiments, the pamoate salt is Form B.

Having now generally described the invention, the same will be more readily understood through reference to the following examples, which are provided by way of illustration and are not intended to be limiting of the present invention.

### EXAMPLES

The disclosure now being generally described, it will be more readily understood by reference to the following examples which are included merely for purposes of illustration of certain aspects and embodiments of the present invention, and are not intended to limit the invention

### General Procedures and Analytical Methods

Reaction Crystallization: About 50 mg of 1-(2-methoxyethyl)-2-methyl-4,9-dioxo-3-(pyrazin-2-ylmethyl)-4,9-dihydro-1H-naphtho[2,3-d]imidazolium bromide was added to a suitable solvent, an equimolar of an acid and NaOH solution or an equimolar of salt was added under stirring at 50° C. for 2 hours and then at 25° C. for at least 12 hours. 95% acetonitrile, 95% methanol and H₂O were used as screening solvents based on solubility of the starting material. For those clear solutions obtained, half volume was evaporated in a fume hood; the rest was treated by anti-solvent addition. Obtained suspensions were taken out and centrifuged. Solids obtained were analyzed by XRPD.

Crystallization by Addition of Anti-Solvent: Form A of the pamoate salt was dissolved in the minimal amount of selected solvents at ambient temperature (about 20-25° C.). 4 folds of anti-solvent was added into the obtained clear solutions slowly until a large amount of solids precipitated out. Precipitates were collected by centrifugation filtration through a 0.45 μm nylon membrane filter at 14,000 rpm. Solid parts (wet cakes) were investigated by XRPD.

Crystallization by Slow Cooling: Form A was dissolved in the minimal amount of selected solvents at 50° C. Obtained solutions were filtered through a 0.45 μm nylon membrane filter by centrifugation at 14,000 rpm. Obtained clear solutions were cooled to 5° C. at 0.1° C./min. Precipitates were collected by centrifugation filtration through a 0.45 μm nylon membrane filter at 14,000 rpm. Solid parts (wet cakes) were investigated by XRPD.

Equilibration Experiments: Form A of the pamoate salt was equilibrated in 0.4-1.0 mL of selected solvents at 25° C. for 2 weeks or at 50° C. for 10 days with a stirring bar on a magnetic stirring plate at a rate of 300-400 rpm. Obtained suspension was filtered through a 0.45 μm nylon membrane filter by centrifugation at 14,000 rpm. Solid parts (wet cakes) were investigated by XRPD.

### Example 1: Synthesis of 1-(2-methoxyethyl)-2-methyl-4,9-dioxo-3-(pyrazin-2-ylmethyl)-4,9-dihydro-1H-naphtho[2,3-d]imidazolium pamoate Form A

Form A was initially obtained from pyridine/water mixture by reaction crystallization with 1 equiv pamoic acid.

20 g scale synthesis: About 20 g of 1-(2-methoxyethyl)-2-methyl-4,9-dioxo-3-(pyrazin-2-ylmethyl)-4,9-dihydro-1H-naphtho[2,3-d]imidazolium bromide and about 1 equivalent of pamoic acid was weighed into a 150 mL glass vial. 70 mL of water/pyridine=8/2(v/v), which contained 1 equivalent of NaOH was added into the vial under stirring at 50° C. Additional 40 mL of water/pyridine=9/1(v/v) was added into the mixture to obtain a better suspension. 83.5 mg of Form A seeds were added into the mixture (suspension). Obtained sample was stirred at 50° C. for about 5 hours, then cooled to 5° C. with a cooling rate of 0.1° C./min and kept stirring at 5° C. for about 12 hours (suspension). Solids were collected by suction filtration and dried at 50° C. under vacuum for about 2 hours. The solids were equilibrated in about 100 mL of ethanol at 25° C. for about 40 hours. Solids were obtained by suction filtration. Then the solids were washed by about 200 mL of water and the suspension was filtered by suction. Obtained solids were dried at 50° C. under vacuum for about 16 hours. About 24.64 g of Form A was obtained as a dark purple solid with a yield of 72.6% and 99.1% purity by HPLC.

Form A was characterized by XRPD, DSC, TGA, ¹H NMR and ion chromatography (IC).

Form A was determined to be an anhydrate having high crystallinity. XRPD spectrum and its corresponding peaks are shown in FIG. 1A and Table 1. FIG. 1B shows DSC thermogram and FIG. 1C shows TGA thermogram of Form A. DSC shows a melting peak at Tₒₙₛₑₜ of 248.2° C. Decomposition occurs upon melting. TGA shows about 0.8% weight loss at about 230° C. ¹H-NMR shows stoichiometry of 1-(2-methoxyethyl)-2-methyl-4,9-dioxo-3-(pyrazin-2-ylmethyl)-4,9-dihydro-1H-naphtho[2,3-d]imidazolium: pamoic acid was 1:1.0 and no detectable residual solvent. IC analysis indicated that residual Br was below limit of quantification (LOQ), i.e., less than 1 mg/L.

Form A converted to Form B in ethanol, acetone, ACN and pyridine/water from competitive experiments.

### Example 2. Synthesis of 1-(2-methoxyethyl)-2-methyl-4,9-dioxo-3-(pyrazin-2-ylmethyl)-4,9-dihydro-1H-naphtho[2,3-d]imidazolium pamoate Form B

About 100 g of 1-(2-methoxyethyl)-2-methyl-4,9-dioxo-3-(pyrazin-2-ylmethyl)-4,9-dihydro-1H-naphtho[2,3-d]imidazolium bromide, batch K1550803, and about 1 equivalent of pamoic acid was weighed into a 5 L glass reactor. 1.2 L of water/pyridine=8/2(v/v), which contained 1 equivalent of NaOH, was added into the vial under stirring at 50° C. Suspension was obtained at 50° C. 0.5 g of Form B was added to the above suspension as seeds at 50° C. The mixture was stirred at 50° C. for about 3 days, then cooled to 25° C. with a cooling rate of 0.2° C./min. 1.2 L of water was added to the suspension as anti-solvent. After stirring at 25° C. for about 6 days, the suspension was cooled to 5° C. with a cooling rate of 0.2° C./min and stirred at 5° C. for about 5 days. Solids were collected by suction filtration and dried at 50° C. under vacuum for about 40 hours. The solids were slurried in 0.5 L of ethanol for about 18 hours and solids were collected by suction filtration. The obtained solids were equilibrated in 700 mL of water at 5° C. for about 3 days. The suspension was filtered and obtained solids were dried at 50° C. under vacuum for about 40 hours. About 125 g of Form B was obtained as a dark purple solid in a yield of 73.8% and 99.5% purity by HPLC.

Form B was characterized by XRPD, DSC, TGA, ¹H NMR and ion chromatography (IC).

Form B was determined to be an anhydrate having high crystallinity. XRPD spectrum and its corresponding peaks are shown in FIG. 2A and Table 2. FIG. 2B shows DSC thermogram and FIG. 2C shows TGA thermogram of Form B. DSC shows a melting peak at Tₒₙₛₑₜ of 248.0° C. Decomposition occurs upon melting. It shows about 0.6% weight loss at about 220° C. by TGA. H-NMR shows stoichiometry of 1-(2-methoxyethyl)-2-methyl-4,9-dioxo-3-(pyrazin-2-ylmethyl)-4,9-dihydro-1H-naphtho[2,3-d]imidazolium: pamoic acid was 1:1.0 and no detectable residual solvent. IC analysis indicated that residual Br was below limit of quantification (LOQ) that is 1 mg/L (equivalent to 0.2% by weight in the 1-(2-methoxyethyl)-2-methyl-4,9-dioxo-3-(pyrazin-2-ylmethyl)-4,9-dihydro-1H-naphtho[2,3-d]imidazolium pamoate salt). The Pattern B is the most stable form among Forms A-J.

### Example 3. Synthesis of 1-(2-methoxyethyl)-2-methyl-4,9-dioxo-3-(pyrazin-2-ylmethyl)-4,9-dihydro-1H-naphtho[2,3-d]imidazolium pamoate ethanol solvate Form C

Form C was obtained from recrystallization by addition of anti-solvent experiment with N,N-Dimethylacetamide (DMAc) as the solvent and ethanol as the anti-solvent. Form C was determined to be an ethanol solvate having medium crystallinity.

Form C was characterized by XRPD, DSC, TGA, and ¹H NMR. XRPD spectrum and its corresponding peaks are shown in FIG. 3A and Table 3. FIG. 3B shows DSC thermogram and FIG. 3C shows TGA thermogram of Form C. About 0.3 equivalent ethanol (1.8% by weight) was detected by ¹H-NMR and stoichiometry of 1-(2-methoxyethyl)-2-methyl-4,9-dioxo-3-(pyrazin-2-ylmethyl)-4,9-dihydro-1H-naphtho[2,3-d]imidazolium: pamoic acid was 1:1.0. DSC shows a desolvation peak at Tₒₙₛₑₜ of 37.3° C. with enthalpy of about 76 J/g, an exothermic peak at Tₒₙₛₑₜ of 170.0° C. with enthalpy of about 20 J/g and melting at Tₒₙₛₑₜ of 237.8° C., combined with decomposition. It shows about 7.2% weight loss at about 200° C. by TGA.

After exposure to ambient condition for about 2 hours and drying at 25° C. under vacuum for about 2 hours, Form C converted to a mixture of Form A and Form B.

### Example 4. Synthesis of 1-(2-methoxyethyl)-2-methyl-4,9-dioxo-3-(pyrazin-2-ylmethyl)-4,9-dihydro-1H-naphtho[2,3-d]imidazolium pamoate NMP solvate Form D

Form D was obtained from crystallization by slow cooling in NMP (N-Methyl pyrrolidone) and equilibration experiments at 25° C. and 50° C. in NMP. Form D was determined to be an NMP solvate having high crystallinity.

Form D was characterized by XRPD, DSC, TGA, and ¹H NMR. XRPD spectrum and its corresponding peaks are shown in FIG. 4A and Table 4. FIG. 4B shows DSC thermogram and FIG. 4C shows TGA thermogram of Form D. About 9.0 equivalents n-methyl pyrrolidone (54.3% by weight) was detected by ¹H-NMR and stoichiometry of 1-(2-methoxyethyl)-2-methyl-4,9-dioxo-3-(pyrazin-2-ylmethyl)-4,9-dihydro-1H-naphtho[2,3-d]imidazolium: pamoic acid was 1:1.0. DSC shows desolvation peaks at Tₒₙₛₑₜ of 10.7° C. with enthalpy of about 48 J/g, at Tₒₙₛₑₜ of 74.7° C. with enthalpy of about 98 J/g, and Tₒₙₛₑₜ of 171.3° C. with enthalpy of about 60 J/g, and melting at Tₒₙₛₑₜ of 243.1° C. combined with decomposition. It shows about 33.2% weight loss at about 140° C. and about 9.5% weight loss from about 140° C. to 220° C. by TGA.

### Example 5. Synthesis of 1-(2-methoxyethyl)-2-methyl-4,9-dioxo-3-(pyrazin-2-ylmethyl)-4,9-dihydro-1H-naphtho[2,3-d]imidazolium pamoate DMAc solvate Form E

Form E was obtained from crystallization by slow cooling in DMAc. Form E was determined to be an DMAc solvate having low crystallinity.

Form E was characterized by XRPD and ¹H NMR. XRPD spectrum and its corresponding peaks are shown in FIG. 5 and Table 5. ¹H-NMR shows about 1.6 equivalents DMAc (15.7% by weight) and stoichiometry of 1-(2-methoxyethyl)-2-methyl-4,9-dioxo-3-(pyrazin-2-ylmethyl)-4,9-dihydro-1H-naphtho[2,3-d]imidazolium: pamoic acid was 1:1.1.

### Example 6. Synthesis of 1-(2-methoxyethyl)-2-methyl-4,9-dioxo-3-(pyrazin-2-ylmethyl)-4,9-dihydro-1H-naphtho[2,3-d]imidazolium pamoate DMAc solvate Form F, Form G, and Form H

Form F, Form G, and Form H were obtained from crystallization by slow cooling in DMAc.

About 300 mg of Form A was weighed into a 40 mL glass vial. 27 mL of DMAc was added into the vial under stirring at 50° C. The mixture was filtered by 0.45 μm nylon filter to obtain a clear solution. The clear solution was cooled to 5° C. at a cooling rate of 0.1° C./min and kept stirring at 5° C. for about 12 hours. Then the solution was cooled to −20° C. at a cooling rate of 0.1° C./min and kept stirring at −20° C. for about 25 hours (clear solution became a suspension).

Form F was obtained as dark purple solid when the suspension was filtered at 14000 rpm for 5 min. Form F was determined to be an DMAc solvate having medium crystallinity. Form F was characterized by XRPD and ¹H NMR. XRPD spectrum and its corresponding peaks are shown in FIG. 6 and Table 6. ¹H-NMR shows about 14.6 equivalents DMAc (62.9% by weight) and stoichiometry of 1-(2-methoxyethyl)-2-methyl-4,9-dioxo-3-(pyrazin-2-ylmethyl)-4,9-dihydro-1H-naphtho[2,3-d]imidazolium: pamoic acid was 1:1.0.

Form G was obtained as dark purple solid when the suspension was filtered at 4000 rpm for 20 min. Form G was determined to be an DMAc solvate having high crystallinity. Form G was characterized by XRPD (FIG. 7 and Table 7).

Form H was obtained as dark purple solid after storing Form G at 25° C. for about 1 day. Form H was determined to be an DMAc solvate having medium crystallinity. Form H was characterized by XRPD, DSC, TGA, and ¹H NMR. XRPD spectrum and its corresponding peaks are shown in FIG. 8A and Table 8. FIG. 8B shows DSC thermogram and FIG. 8C shows TGA thermogram of Form H. About 13.0 equivalents DMAc (60.1% by weight) was detected by ¹H-NMR and stoichiometry of 1-(2-methoxyethyl)-2-methyl-4,9-dioxo-3-(pyrazin-2-ylmethyl)-4,9-dihydro-1H-naphtho[2,3-d]imidazolium: pamoic acid is 1:1.0. DSC shows desolvation peaks at Tₒₙₛₑₜ of 56.6° C. with enthalpy of about 98 J/g, and at Tₒₙₛₑₜ of 117.3° C. with enthalpy of about 45 J/g and melting at Tₒₙₛₑₜ of 246.6° C. combined with decomposition. It shows about 54.8% weight loss at about 97° C. and about 5.3% weight loss from about 97° C. to 125° C. by TGA.

After heating to 170° C., Form H converted to a mixture of Form A and Form B.

### Example 7. Synthesis of 1-(2-methoxyethyl)-2-methyl-4,9-dioxo-3-(pyrazin-2-ylmethyl)-4,9-dihydro-1H-naphtho[2,3-d]imidazolium pamoate HFIPA solvate Form I

Form I was obtained from hexafluoroisopropyl acrylate (HFIPA) by equilibration experiment at 25° C. Form I was determined to be an HFIPA solvate having low crystallinity.

Form I was characterized by XRPD, DSC, TGA, and ¹H NMR. XRPD spectrum and its corresponding peaks are shown in FIG. 9A and Table 9. FIG. 9B shows DSC thermogram and FIG. 9C shows TGA thermogram of Form I. About 1.0 equivalent HFIPA (8.9% by weight) was detected by ¹H-NMR and stoichiometry of 1-(2-methoxyethyl)-2-methyl-4,9-dioxo-3-(pyrazin-2-ylmethyl)-4,9-dihydro-1H-naphtho[2,3-d]imidazolium: pamoic acid was 1:3.5. DSC shows desolvation peaks at Tₒₙₛₑₜ of 25.3° C. with enthalpy of about 21 J/g and at Tₒₙₛₑₜ of 88.0° C. with enthalpy of about 11 J/g as well as melting at Tₒₙₛₑₜ of 281.5° C. combined with decomposition. It shows about 2.3% weight loss at about 80° C. and about 8.0% weight loss from about 80° C. to 200° C. by TGA.

### Example 8. Synthesis of 1-(2-methoxyethyl)-2-methyl-4,9-dioxo-3-(pyrazin-2-ylmethyl)-4,9-dihydro-1H-naphtho[2,3-d]imidazolium pamoate DMF solvate Form J

Form J was obtained from crystallization by slow cooling in N,N-Dimethylformamide (DMF). About 300 mg of Form A was weighed into a 40 mL glass vial. 30 mL of DMF was added into the vial under stirring at 50° C. The mixture was filtered by 0.45 μm nylon filter to obtain a clear solution. The clear solution was cooled to −20° C. at a cooling rate of 0.1° C./min. And then the clear solution was stirred at −20° C. for about 40 hours and stored at −20° C. for about 3 days. The clear solution converted into a suspension. Solids were collected by centrifugation filtration. Form J was obtained as a sticky solid.

Form J was determined to be a DMF solvate having high crystallinity. Form J was characterized by XRPD, DSC, TGA, and ¹H NMR. XRPD spectrum and its corresponding peaks are shown in FIG. 10A and Table 10. FIG. 10B shows DSC thermogram and FIG. 10C shows TGA thermogram of Form J. About 6.3 equivalents DMF (38.0% by weight) was detected by ¹H-NMR and stoichiometry of 1-(2-methoxyethyl)-2-methyl-4,9-dioxo-3-(pyrazin-2-ylmethyl)-4,9-dihydro-1H-naphtho[2,3-d]imidazolium: pamoic acid was 1:1.0. DSC shows desolvation peaks at Tₒₙₛₑₜ of 28.6° C. with enthalpy of about 174 J/g, at Tₒₙₛₑₜ of 86.2° C. with enthalpy of about 18 J/g, and at Tₒₙₛₑₜ of 153.6° C. with enthalpy of about 24 J/g, and melting at Tₒₙₛₑₜ of 248.7° C. combined with decomposition. It shows about 0.4% weight loss at about 100° C. and about 9.5% weight loss from about 100° C. to 200° C. by TGA.

### Example 9. Competitive Equilibration Experiments of Form A and Form B of the Pamoate Salt

About 5 mg of Form A and about 5 mg of the mixture of Form A and Form B were added to 0.2 mL saturated solutions in following solvents: water, ethanol, acetone, acetonitrile, ethyl acetate, and pyridine/water (1/9 v/v). Obtained suspensions were stirred at 25° C. and 50° C., respectively, for a duration between 4 days to 4 weeks. Solid parts (wet cakes) were isolated by centrifugation filtration and investigated by XRPD.

Pure Form B was obtained from pyridine/water (25° C. and 50° C.), ethanol (25° C. and 50° C.), acetone (50° C.) and acetonitrile (25° C. and 50° C.). The other experiments resulted in mixture of Form A and Form B.

### Example 10. Solid-State Thermal Stability of the Pamoate Salt Form B

Form B was placed at 25° C./92% relative humidity (RH) in an open container, at 40° C./75% RH in an open container, and at 60° C. in a tight container for 1 week. Samples were characterized by XRPD and HPLC and inspected for appearance including color change. Results are shown in Table 11. Form B is physically stable and chemically stable under tested conditions.

TABLE 11
Stability Results

| | | 25° C./ | 40° C./ | 60° C. |
|---|---|---|---|---|
| | | 92% RH | 75% RH | in a tight |
| | | open container, | open container, | container, |
| | Initial | 1 week | 1 week | 1 week |
| Purity by HPLC | 99.3% | 99.4% | 99.5% | 99.4% |
| XRPD | Form B | Form B | Form B | Form B |
| Appearance | None | None | None | none |
| (Color) change | | | | |

### Example 11. Solubility of the Pamoate Salt Form a and Form B

19 mg or 4.2 mg of Form A and Form B were weighed into a glass vial. 5 mL of water and PBS buffer or 2 mL mouse plasma was added. Obtained suspensions were stirred at 37° C. at 400 rpm for 24 hours and then centrifuged at 37° C. at 14,000 rpm for 5 min. Supernatants were analyzed by HPLC and pH meter for solubility and pH, respectively. Residual solids (wet cakes) were characterized by XRPD. Results arc shown in Table 12.

Form B and Form A show comparable low solubility. According to XRPD after solubility test, there is no form change for both Form B and Form A.

TABLE 12
Solubility Results

| | Form A | | | Form B | | |
|---|---|---|---|---|---|---|
| | Solubility (pH) | | XRPD | Solubility (pH) | | XRPD |
| water | 0.012 mg/mL | 0.013 mg/mL | Form A | 0.014 mg/mL | 0.013 mg/mL | Form B |
| | (7.43) | (7.32) | | (7.38) | (7.46) | |
| pH 7.4, | 0.084 mg/mL | 0.086 mg/mL | Form A | 0.078 mg/mL | 0.078 mg/mL | Form B |
| PBS | (7.30) | (7.31) | | (7.31) | (7.32) | |
| buffer | | | | | | |
| mouse | 0.436 mg/mL | 0.403 mg/mL | Form A | 0.342 mg/mL | 0.344 mg/mL | Form B |
| plasma | | | | | | |

### Example 12. Evaluation of Hygroscopicity of Form B by Dynamic Vapor Sorption

Water sorption and desorption behavior of Form B was investigated by dynamic vapor sorption (DVS) at 25° C. with a cycle of 40-0-95-0-40% RH, dm/dt 0.002, minimum equilibration time of 60 min and maximum equilibration time 360 min. Results are shown in Table 13.

TABLE 13
DVS Results

| | 1st | 1st | 2nd | 2nd |
|---|---|---|---|---|
| Relative | desorption | sorption | desorption | sorption |
| humidity at | weight % | weight % | weight % | weight % |
| 25° C. | change | change | change | change |
| 0.0 | 0.02 | 0.02 | 0.00 | 0.00 |
| 10.0 | 0.06 | 0.06 | 0.05 | 0.04 |
| 20.0 | 0.09 | 0.08 | 0.08 | 0.06 |
| 30.0 | 0.13 | 0.11 | 0.10 | 0.08 |
| 40.0 | 0.16 | 0.12 | 0.13 | 0.10 |
| 50.0 | N/A | 0.15 | 0.16 | N/A |
| 60.0 | N/A | 0.17 | 0.18 | N/A |
| 70.0 | N/A | 0.20 | 0.21 | N/A |
| 80.0 | N/A | 0.23 | 0.25 | N/A |
| 90.0 | N/A | 0.35 | 0.38 | N/A |
| 95.0 | N/A | 0.64 | 0.64 | N/A |

Form B was slightly hygroscopic. It absorbs about 0.64% water up to 95% RH at 25° C. After the DVS test, the sample was still Form B by XRPD.

### Example 13. Compression, Dry Grinding, and Wet Granulation Simulation Experiments

Compression: About 50 mg of Form B was compressed for 5 minutes under 10 MPa with a hydraulic press. Potential form change and degree of crystallinity were evaluated by XRPD. Form B showed good tolerance to compression with no form change and no obvious crystallinity decrease.

Dry Grinding: About 20 mg of Form B was ground manually with a mortar and a pestle for 5 min. Potential form change and degree of crystallinity were evaluated by XRPD. Form B showed good tolerance to dry grinding granulation with no form change and slight crystallinity decrease.

Wet Granulation: Water or ethanol was added drop wise to about 20 mg of Form B until the sample was wetted sufficiently. Wet sample was ground gently with a mortar and a pestle for 3 min. Post granulation sample was dried under ambient condition for 10 min. Potential form change and degree of crystallinity were evaluated by XRPD. Form B showed good tolerance to water and ethanol granulation with no form change and no obvious crystallinity decrease.

### Example 14. C-Myc and Caspase-3 Protein Expression in Xenografted SU-DHL-10 Tumor

Changes of c-Myc and Caspase-3 protein expression in xenografted SU-DHL-10 tumor after administration of 1-(2-methoxyethyl)-2-methyl-4,9-dioxo-3-(pyrazin-2-ylmethyl)-4,9-dihydro-1H-naphtho[2,3-d]imidazolium pamoate (pamoate) Form A was investigated in vivo. C-Myc expression in tumors was inhibited after administration of the Form A pamoate at 24-hour post dosing compared with vehicle control group, while increased cleaved Caspase-3 protein expression indicated apoptosis of tumor cells.

The pamoate was administrated via single intramuscular (i.m.) injection to SU-DHL-10 xenografted mice to examine the effect of the pamoate on c-Myc expression level in tumor at the 24-hour time point post injection.

The pamoate was mixed with appropriate volume of vehicle solution (0.5% carboxymethylcellulose sodium (CMC-Na, viscosity 400-800 cps, CAS No. 9004-32-4, Sigma-Aldrich, USA) and 0.2% Tween-80 in 10 mM pH 7.4 phosphate buffer saline (PBS)) to get a uniform suspension after 15 min sonication, with final concentration of 5.6 mg/mL. The dose volume was determined by the mice′ body weight collected on the morning of dosing day, calculated by the formula: dose volume (2 mL/kg)×body weight.

Human diffuse large B cell lymphoma cell line SU-DHL-10 was obtained from Cobioer Biotechnology Co., Ltd (China). The cells were cultured at 37° C. with 5% CO₂ in RPMI-1640 (Hyclone, USA) supplemented with 10% heat inactivated fetal bovine serum (Gibco, USA) and 1% GlutaMAX (Gibco, USA). The cells were collected, suspended in PBS at 2×10⁸ cells/mL, and then mixed with the equivalent volume of Matrigel® Basement Membrane Matrix (Corning, USA).

Six-week-old female BALB/c-nude mice were purchased from SPF Biotechnology Co., Ltd. (Beijing, China). They were maintained on a standard diet and water throughout the experiments under specific pathogen-free conditions. The cells were cultured in vitro and 0.1×10⁸ cells/0.1 mL/mouse were grafted subcutaneously into the flank of NOD-SCID (NVSG) mice (Beijing Viewsolid Biotech Co. Ltd), 6-week-old. When the tumor reached about 1 cm in diameter, tumor-bearing mice were sacrificed, and tumors were removed and sliced into small fragments (approximately 3×3×3 mm³/fragment) and re-inoculated into BALB/c nude mice. Drug administration was performed when tumor volume was greater than 500 mm³.

Experimental groups were treated as Table 14. The pamoate or vehicle solution was administered only once. Tumors were collected from the animals after 24 hours post dosing, and then frozen at −80° C. until use.

TABLE 14
Study Design

| Group | Test | No. of | Dose | Dose | |
|---|---|---|---|---|---|
| No. | Substance | Animals | Route | (mg/kg) | Vehicle |
| 1 | Vehicle | 3 | i.m. | 0 | 0.5% CMC-Na, |
| | | | | | 0.2% Tween- |
| | | | | | 80 in 10 mM |
| | | | | | pH 7.4 PBS saline |
| 2 | Pamoate | 3 | i.m. | 11.2 | 0.5% CMC-Na, |
| | | | | | 0.2% Tween- |
| | | | | | 80 in 10 mM |
| | | | | | pH 7.4 PBS saline |

The tumor was excised with scissors, resuspended in cold cell lysis buffer containing 20 mM Tris (pH7.5), 150 mM NaCl, 1% Triton X-100, 2 mM AEBSF, 0.3 M Aprotinin, 130 μM Bestatin, 14 μM E64, 10 μM Leupeptin, 5 mM sodium fluoride, 1 mM sodium pyrophosphate, 1 mM β-glycerophosphate and 1 mM sodium orthovanadate, and then homogenized with handheld homogenizer (Beyotime, China). The homogenates were kept on ice for 30 minutes followed by centrifugation (4° C., 12,000 rpm, 20 minutes) to obtain a clear lysate. Protein contents were determined using Micro BCA™ Protein Assay Kit (23235, Thermo Scientific, USA). Aliquots of 40 μg proteins were boiled in 1×SDS sample loading buffer for 10 minutes at 99° C. and separated by SDS-PAGE, and then subjected to Western blot analysis with anti-c-Myc antibody (ab32072, Abeam, USA), Caspase-3 rabbit polyclonal antibody (19677-1-AP, Proteintech, USA) and anti-GAPDH antibody (TA-08, ZSGB-BIO, China). Visualization was carried out using the Enhanced Chemi-luminescence Kit (Beyotime, China).

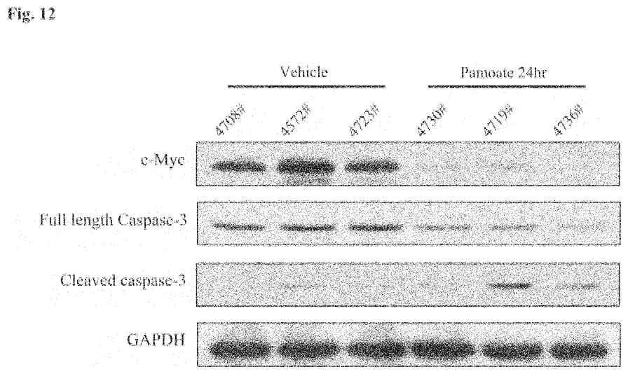

Changes of c-Myc and Caspase-3 protein expression in xenografted SU-DHL-10 tumor after administration of the pamoate was investigated in vivo (FIG. 12). There was no change in GAPDH protein contents between vehicle group and the pamoate treatment group. In comparison, c-Myc expression in tumors was inhibited after administration of the pamoate for 24 hours. The pamoate was found to produce a drastic decrease of c-Myc protein level in xenografted SU-DHL-10 tumor. Full length Caspase-3 expression was decreased while cleaved Caspase-3 expression increased which indicated tumor cell apoptosis induced by the pamoate. In FIG. 12, cell lysates equivalent to 40 g of total protein were loaded on each lane and the lysates were analyzed by Western blotting with antibodies against c-Myc, Caspase-3 and GAPDH.

In this study, solution concentrations and dosing levels are all expressed in terms of 1-(2-methoxyethyl)-2-methyl-4,9-dioxo-3-(pyrazin-2-ylmethyl)-4,9-dihydro-1H-naphtho[2,3-d]imidazolium, the cationic moiety of the test substance.

### Example 15. Anti-Tumor Activity

The anti-tumor activities of 1-(2-methoxyethyl)-2-methyl-4,9-dioxo-3-(pyrazin-2-ylmethyl)-4,9-dihydro-1H-naphtho[2,3-d]imidazolium monobromide (monobromide, or monobromide salt) and 1-(2-methoxyethyl)-2-methyl-4,9-dioxo-3-(pyrazin-2-ylmethyl)-4,9-dihydro-1H-naphtho[2,3-d]imidazolium pamoate salt (pamoate, or pamoate salt) Form A against SU-DHL-10 human diffuse large B cell lymphoma xenograft were examined.

The 1-(2-methoxyethyl)-2-methyl-4,9-dioxo-3-(pyrazin-2-ylmethyl)-4,9-dihydro-1H-naphtho[2,3-d]imidazolium monobromide is considered to be a promising potent anti-tumor agent candidate; however, its rapid clearance from plasma after i.v. administration limited the clinical use. At present, 7-day continuous i.v. infusion was applied in order to maintain plasma concentration, but this was not a patient-friendly administration method. The Form A pamoate has the same active ingredient as the monobromide, but its water solubility is extremely low. The extended-release characteristics of pamoate, due to it low solubility, was examined.

The monobromide used in this study had a purity of 99.6% and a salt factor of 1.22. The pamoate salt used in this study had a purity of 99.7% and a salt factor of 2.07. For both drug substances, solution concentrations and dosing levels are all expressed in terms of 1-(2-methoxyethyl)-2-methyl-4,9-dioxo-3-(pyrazin-2-ylmethyl)-4,9-dihydro-1H-naphtho[2,3-d]imidazolium, the cationic moiety of the test substances.

For the monobromide continuous i.v. infusion group, the monobromide was dissolved with physiological saline to prepare 12.12 mg/mL clear solution. A micro-osmotic pump was used (Alzet®, Model 1004, DURECT corporation, USA) with pumping rate of 0.11 μl per hour, achieving an average daily release of 32 μg monobromide, hence the daily dosage is approximately 1.6 mg/kg/day based on 20 g mouse body weight.

For the monobromide i.p. injection group, the monobromide was dissolved with physiological saline to prepare 0.41 mg/mL clear solution. The dose volume was determined by the mice′ body weight collected on the morning of dosing day, calculated by the formula: dose volume (10 mL/kg)×body weight.

For the pamoate i.m. injection group, the pamoate salt was mixed with appropriate volume of vehicle solution (0.5% CMC-Na (viscosity 400-800 cps, CAS No. 9004-32-4), 0.2% Tween-80 in 10 mM pH 7.4 PBS saline) to get a uniform suspension after 15 min sonication, with final concentration of 5.6 mg/mL, similar to the procedure described in Example 14. The dose volume was determined by the mice′ body weight collected on the morning of dosing day, calculated by the formula: dose volume (2 mL/kg)×body weight.

Human diffuse large B cell lymphoma cell line SU-DHL-10 was obtained from Cobioer Biotechnology Co., Ltd (China). The cells were cultured at 37° C. with 5% CO₂ in RPMI-1640 (Hyclone, USA) supplemented with 10% heat inactivated fetal bovine serum (Gibco, USA) and 1% GlutaMAX (Gibco, USA). The cells were collected, suspended in PBS at 2×10⁸ cells/mL, and then mixed with the equivalent volume of Matrigel® Basement Membrane Matrix (Corning, USA).

Six-week-old female BALB/c-nude mice were purchased from SPF Biotechnology Co., Ltd. (Beijing, China). They were maintained on a standard diet and water throughout the experiments under specific pathogen-free conditions. The cells were cultured in vitro and 0.1×10⁸ cells/0.1 mL/mouse were grafted subcutaneously into the flank of NOD-SCID (NVSG) mice (Beijing Viewsolid Biotech Co. LTD), 6-week-old. When the tumor reached about 1 cm in diameter, tumor-bearing mice were sacrificed and tumors were removed and sliced into small fragments (approximately 3×3×3 mm³/fragment) and re-inoculated into BALB/c nude mice. The mice with a tumor volume (length×width²×0.5) of 99.28 to 247.10 mm³ were divided into four groups randomly, using Study Director Software (Studylog Systems, Inc, Version 3.1.399.22, USA) to minimize intragroup and intergroup tumor volume variation.

The first day of administration was designated day 0, and observation continued until day 35. Each group was treated as follows (Table 15).

TABLE 15
Study Design

| | | | | | | Dose | | |
|---|---|---|---|---|---|---|---|---|
| Group | Test | No. of | Dose | Dose | Conc. | Volume | | |
| No. | Substance | Animals | Route | (mg/kg) | (mg/mL) | (mL/kg) | Vehicle | Dose Time |
| 1 | Vehicle | 5 | i.m. | 0 | 0 | 2 | 0.5% CMC-Na, 0.2% | Day 0, 7, 14 |
| | | | | | | | Tween-80 in 10 mM pH | |
| | | | | | | | 7.4 PBS saline | |
| 2 | Monobromide | 6 | i.v. | 1.6* | 12.12 | NA** | physiological saline | Day 0-21 |
| | salt | | | | | | | continuous i.v. |
| | | | | | | | | infusion |
| 3 | Monobromide | 6 | i.p. | 4.1 | 0.41 | 10 | physiological saline | day 0-4, 7-11, |
| | salt | | | | | | | 14-18 |
| 4 | Pamoate salt | 6 | i.m. | 11.2 | 5.6 | 2 | 0.5% CMC-Na, 0.2% | Day 0, 7, 14 |
| | | | | | | | Tween-80 in 10 mM pH | |
| | | | | | | | 7.4 PBS saline | |

*Daily dose of Group 2 was approximately 32 μg per mouse. 1.6 mg/kg was calculated based on 20 g mouse body weight.
**The pumping rate of osmotic pump was 0.11 μL per hour, with daily dose volume of 2.64 μL.

The vehicle control group received i.m. injection of vehicle solution on day 0, 7 and 14 (Group 1). For 21-day continuous i.v. infusion, an osmotic pump containing the monobromide was implanted in the dorsum of each animal while under anesthesia on day 0, with daily dose of 32 g, approximately 1.6 mg/kg/day (Group 2). The monobromide i.p. injection group was administrated referring to FIVE-DAY-ON, TWO-DAY-OFF procedure on day 0-4, 7-11 and 14-18 (Group 3) per Minoda et. al, Antitumor effect of YM155, a novel small-molecule survivin suppressant, via mitochondrial apoptosis in human MFH/UPS, International Journal of Oncology, 2015, 47:891-899. For the pamoate i.m. injection group, drug suspension was administrated once per week at 11.2 mg/kg, approximately 7 times daily dosage of the monobromide i.v. infusion group, on day 0, 7 and 14 (Group 4).

Body weight was measured during administration, and tumor diameter was measured twice a week from day 0 to day 35. The anti-tumor activities were expressed as percent inhibition of tumor growth (% inh) and percent regression of the tumor (% reg). The percent inhibition of tumor growth was calculated for treated group using the following formula: 100×{1−[(mean tumor volume of treated group)−(mean tumor volume of treated group on day 0)]/[(mean tumor volume of the control group)−(mean tumor volume of the control group on day 0)]}. The percent regression of the tumor (% reg) was calculated for all groups with observed tumor regression, using the following formula: 100×[1−(mean tumor volume of treated group)/(mean tumor volume of treated group on day 0)]. Mice were euthanized when the tumor load exceeded 3000 mm³ or when the average tumor volume in a group exceeded 2500 mm³.

Data were expressed as mean±standard error of the mean. Tumor volume and body weight on day 35 were compared between each group using T-test, in which P values of less than 5% were considered significant. For any group with observed tumor regression, paired t-test was performed to compare their tumor volumes, between day 0 and day 35. In this case, P values of less than 5% were considered significant. Study Director Software (Studylog Systems, Inc. Version 3.1.399.22, USA) was used for data processing.

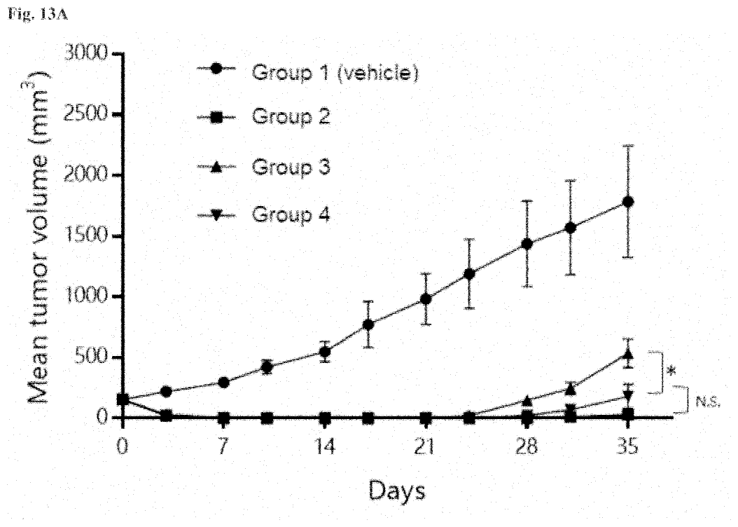

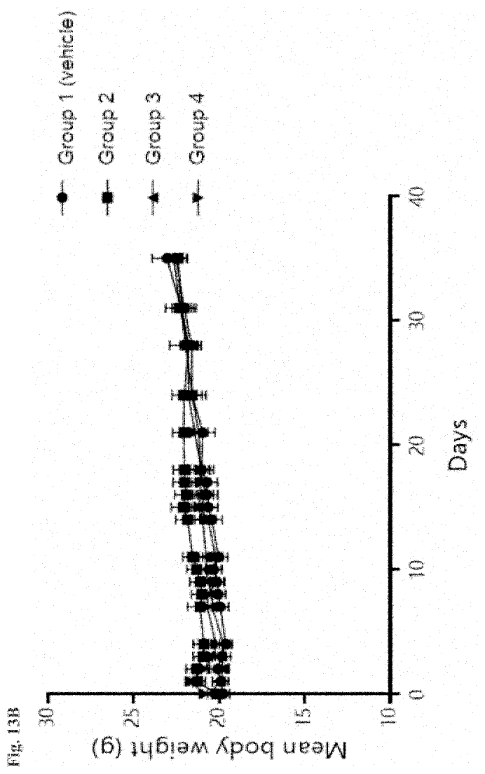

The anti-tumor activities of the monobromide and the pamoate against SU-DHL-10 xenograft were evaluated until day 35 (Tables 16A-16B and FIGS. 13A-13B). In Tables 16A-16B, values are expressed as mean SEM (Group 1, n=5; Group 2-4, n=6). The antitumor activities are expressed as percentage inhibition of tumor growth (% inh) and percentage regression of the tumor (% reg); 0% reg means no change in tumor volume, and 100% reg means complete remission of tumor. In FIGS. 13A-13B, statistical analysis was performed for the values on day 35. *, P<0.01 versus Group 4; ^{N.S.}, no significant from Group 4. (Student's t-test).

The monobromide (clear solution) was administrated via 21-day continuous i.v. infusion or i.p. injection, while the pamoate was prepared as a sustained release formulation (uniform suspension), administrated once per week via i.m. injection. After 21-day treatment, 100% tumor regression was achieved in all experimental groups. However, tumor recurrence occurred during observation continued until day 35. Groups 2, 3, and 4 inhibited tumor growth by 107.67%, 76.78%, and 98.22% on day 35, respectively. Group 4 demonstrated considerable anti-tumor efficacy similar to Group 2 on day 28 and day 35 (no statistical significant difference in tumor volume), and efficacy of Group 4 was much better than Group 3 (P<0.05). Each group demonstrated good safety profiles, and no statistically significant decrease in body weight was observed. The final complete response (CR) rates were 3/6, 0/6, 3/6 for Group 2, Group 3, and Group 4, respectively. This study indicates that the pamoate salt has good safety and efficacy in the SU-DHL-10 xenograft tumor model.

TABLE 16A
Mean Tumor Volume

| Group | Mean Tumor Volume ± SEM (mm³) | | | | |
|---|---|---|---|---|---|
| No. | Day 0 | Day 7 | Day 21 | Day 28 | Day 35 |
| 1 | 156.08 ± 17.16 | 295.48 ± 75.87 | 983.85 ± 468.63 | 1439.97 ± 353.35 | 1787.28 ± 461.27 |
| 2 | 155.72 ± 14.71 | 0.00 ± 0.00 | 0.00 ± 0.00 | 0.00 ± 0.00 | 30.66 ± 13.72 ** |
| 3 | 156.93 ± 15.69 | 5.20 ± 3.29 | 0.00 ± 0.00 | 149.44 ± 36.72 **^{, ##} | 535.72 ± 116.93 *^{, ##} |
| 4 | 151.26 ± 23.65 | 0.00 ± 0.00 | 0.00 ± 0.00 | 23.61 ± 23.61 **^{, N.S., $} | 180.32 ± 101.25 **^{, N.S., $} |

* P < 0.05 versus vehicle control group;
** P < 0.01 versus vehicle control group.
^{N.S.} no significant from Group 2;
^{##} P < 0.01 versus Group 2.
^{$} P < 0.05 versus vehicle Group 3.

TABLE 16B
Mean Body Weight and Antitumor Activity

| | Mean Body | | Antitumor Activity | | Number of |
|---|---|---|---|---|---|
| Group | Weight ± SEM (g) | | (% inh/% reg) | | CRs |
| No. | Day 21 | Day 35 | Day 21 | Day 35 | Day 35 |
| 1 | 20.98 ± 0.73 | 23.06 ± 0.87 | NA | NA | NA |
| 2 | 22.07 ± 0.66 | 22.42 ± 0.57 | 118.81/100 | 107.67/80.31 | 3/6 |
| 3 | 21.20 ± 0.55 ^{N.S.} | 22.53 ± 0.62 ^{N.S.} | 118.96/100 | 76.78/−241.38 | 0/6 |
| 4 | 21.43 ± 0.35 ^{N.S.} | 22.65 ± 0.30 ^{N.S.} | 118.27/100 | 98.22/−19.21 | 3/6 |

^{N.S.} no significant from Group 2

### Example 16. Photostability Test

1-(2-Methoxyethyl)-2-methyl-4,9-dioxo-3-(pyrazin-2-ylmethyl)-4,9-dihydro-1H-naphtho[2,3-d]imidazolium monobromide (the “monobromide”) and 1-(2-methoxyethyl)-2-methyl-4,9-dioxo-3-(pyrazin-2-ylmethyl)-4,9-dihydro-1H-naphtho[2,3-d]imidazolium pamoate salt (the “pamoate”; Form B) were placed in a petri dish (covered with clear wrap film) and exposed to ID65 light (ICH Option 1 per “ICH Topic QIB Photostability Testing of New Active Substances and Medicinal Products”) until total light exposure reached 1.44 million lux light quantity. Purity via HPLC, XRPD, and appearance inspection were performed before and after light exposure to determine photostability of the two compounds.

The monobromide is a yellow crystalline, which was proved to be photo-unstable in a preliminary study. Degradation occurred after 2 months of light exposure (D65 light, 1000 lux, 25° C./60% RH) for the monobromide, as evidenced by a color change from yellow to yellowish brown and a slight increase in HPLC peak areas, attributed to degradation products (Table 17).

TABLE 17
Photostability of Monobromide

| | | | | | | Related | | | | | |
|---|---|---|---|---|---|---|---|---|---|---|---|
| | | | | | | Substances | | Water | Assay | | |
| Storage | Storage | | | | XRPD | (HPLC, %) | | content | (%) | | Thermal |
| Conditions | Period | Description | UV | IR | pattern | Each max | Total | (%) | HPLC | Titration | behavior |
| Initial | 0 | Yellow | a | a | a | 0.08 | 0.08 | 0.05 | 100.9 | 100.0 | b |
| | | crystalline | | | | | | | | | |
| | | powder | | | | | | | | | |
| D65 light, | 1 | Yellowish | NC | NC | NC | 0.13 | 0.22 | 0.06 | 100.0 | 100.2 | NC |
| 1000 lux, | | brown | | | | | | | | | |
| 25° C./60% | | crystalline | | | | | | | | | |
| relative | | powder | | | | | | | | | |
| humidity | 2 | Yellowish | NC | NC | NC | 0.19 | 0.49 | 0.07 | 99.5 | 100.1 | NC |
| | | brown | | | | | | | | | |
| | | crystalline | | | | | | | | | |
| | | powder | | | | | | | | | |

a Conforms to the reference
b Conforms to Typical DSC/TGA curves as the reference standard
NC: No change

The test substance information is listed in Table 18.

TABLE 18
Test Substance Information

| Test Substance | M.W. | F.W. | Formula | Purity (%) | Appearance |
|---|---|---|---|---|---|
| Monobromide | 363.39 | 443.29 | C₂₀H₁₉N₄O₃•Br | 99.6 | Yellow crystalline |
| | | | | | powder |
| Pamoate | 363.39 | 750.75 | C₄₃H₃₄N₄O₉ | 99.5 | Purple crystalline |
| (Form B) | | | | | powder |

### Photostability Testing:

Light source—ICH Option 1 light source standard was adopted. ID65 light, equivalent indoor indirect daylight standard, with 250 W/m² light source intensity was used.

Total light exposure and light exposure time−total light exposure was set to 1.44 million lux light quantity (1000 lux×24 h×60 d=1,440,000 lux light quantity). Light stability testing box with ID65 light source of 250 W/m² intensity was adopted. According to ICH Option 1 standard, 21.8 hours light exposure could reach 1.2 million lux light quantity. Therefore, 26.2 hours light exposure is needed to reach 1.44 million lux quantity.

Testing—Accurately weight 0.5 g of the monobromide and the pamoate, place in petri dishes (covered with clear wrap film) and spread into a layer no more than 3 mm thickness, separately. Samples were put into light stability testing box, and received total amount of light exposure not less than 1.44 million lux light quantity (ID65 light, 250 W/m², 26.2 hours, 25° C., 60% relative humidity). After light exposure, samples were removed for characterization, including appearance inspection, XRPD and purity via HPLC.

The results of characterization of samples before and after light exposure are shown in Table 19. There was no significant change in the appearance, XRPD and HPLC purity of the pamoate after ICH Option 1 irradiation. However, the color of the monobromide obviously changed from the original yellow crystalline powder to dark purple crystalline powder and the HPLC purity was reduced by 6.2%, while XRPD result had no significant change.

TABLE 19
Photostability Results

| | | Irradiation | | | |
|---|---|---|---|---|---|
| Test | | Time | | | Purity % |
| Substance | Condition | (hours) | Description | XRPD | (HPLC) |
| Monobromide | Initial | 0 | Yellow | NA | 99.81 |
| | | | crystals | | |
| | ID65 light, 250 | 26.2 | Dark Purple | Consistent | 93.61 |
| | W/m², | | crystalline | with initial | |
| | 25° C./60% RH | | powder | | |
| | (ICH Option 1) | | | | |
| Pamoate | Initial | 0 | Purple | NA | 99.29 |
| | | | crystalline | | |
| | | | powder with | | |
| | | | lumps | | |
| | ID65 light, 250 | 26.2 | Purple | Consistent | 99.38 |
| | W/m², | | crystalline | with initial | |
| | 25° C./60% RH | | powder with | | |
| | (ICH Option 1) | | lumps | | |

The monobromide exhibited severe photodegradation, as evidenced by the change in appearance from yellow crystalline powder to dark purple crystalline powder, as well as a decrease in purity from 99.81% to 93.61%. The pamoate exhibited excellent photostability, and its appearance and purity remained almost unchanged before and after irradiation. The results indicate that the pamoate salt has surprisingly much better photostability than the monobromide salt. The Examples herein thus demonstrate that the pamoate salt and embodiments of the present invention not only provide an efficacious and longer release profile than the sepantroium bromide, thereby allowing for a more desirable mode of administration for the patient, but also unexpectedly provide a substantially more stable form, allowing for significant easier handling and storage of the drug product.

What is claimed is:

1. A pamoate salt, 1-(2-methoxyethyl)-2-methyl-4,9-dioxo-3-(pyrazin-2-ylmethyl)-4,9-dihydro-1H-naphtho[2,3-d]imidazolium pamoate, or a solvate thereof.

2. The pamoate salt of claim 1, wherein the pamoate salt is an anhydrate.

3. The pamoate salt of claim 1, wherein the pamoate salt is a solvate.

4. The pamoate salt of claim 3, wherein the solvate is an ethanol solvate, an N-methyl pyrrolidone solvate, an N,N-dimethylacetamide solvate, a hexafluoroisopropyl acrylate solvate, or an N,N-dimethylformamide solvate.

5. The pamoate salt of any one of claims 1-4, wherein the pamoate salt or the solvate is crystalline.

6. The pamoate salt of any one of claims 1-4, wherein the pamoate salt or the solvate is amorphous.

7. The pamoate salt of any one of claims 1-6, wherein the pamoate salt or the solvate has a purity of about 90% or higher.

8. The pamoate salt of any one of claims 1-6, wherein the pamoate salt or the solvate has a purity of about 95% or higher.

9. The pamoate salt of any one of claims 1-6, wherein the pamoate salt or the solvate has a purity of about 98% or higher.

10. The pamoate salt of any one of claims 1-9, wherein the pamoate salt comprises less than 0.5% of bromide ion (Br) by weight.

11. The pamoate salt of any one of claims 1-10, wherein the pamoate salt or solvate thereof is purified and isolated.

12. A pharmaceutical composition comprising any one of the pamoate salt of claims 1-11 and a pharmaceutically acceptable excipient or carrier.

13. A pharmaceutical composition comprising the pamoate salt of claim 10 and a pharmaceutically acceptable excipient or carrier.

14. A crystalline form of a pamoate salt or a solvate thereof, wherein the pamoate salt is 1-(2-methoxyethyl)-2-methyl-4,9-dioxo-3-(pyrazin-2-ylmethyl)-4,9-dihydro-1H-naphtho[2,3-d]imidazolium pamoate.

15. The crystalline form of claim 14, wherein the pamoate salt is an anhydrate.

16. The crystalline form of claim 15, wherein the pamoate salt anhydrate exhibits an X-ray powder diffraction (XRPD) pattern comprising peaks at about 8.08±0.2, 9.69±0.2, and 10.67±0.2 degrees two-theta.

17. The crystalline form of claim 16, wherein the XRPD pattern further comprises peaks at 11.41±0.2, 15.29±0.2, and 16.17±0.2 degrees two-theta.

18. The crystalline form of any one of claims 15-17 which exhibits an XRPD pattern substantially similar to FIG. 1A.

19. The crystalline form of any one of claims 15-18 which exhibits a differential scanning calorimetry (DSC) thermogram comprising an endotherm peak which onsets at about 248° C.

20. The crystalline form of any one of claims 15-19 which exhibits weight percent loss of about 0.8% between about 35° C. to about 230° C. by a thermogravimetric analysis (TGA).

21. The crystalline form of claim 15, wherein the pamoate salt anhydrate exhibits an X-ray powder diffraction (XRPD) pattern comprising peaks at about 9.32±0.2 and 16.89±0.2 degrees two-theta.

22. The crystalline form of claim 21, wherein the XRPD pattern further comprises peaks at 10.97±0.2, 11.60±0.2, and 24.77±0.2 degrees two-theta.

23. The crystalline form of any one of claims 15, 21, and 22 which exhibits an XRPD pattern substantially similar to FIG. 2A.

24. The crystalline form of any one of claims 15, and 21-23 which exhibits a differential scanning calorimetry (DSC) thermogram comprising an endotherm peak which onsets at about 248° C.

25. The crystalline form of any one of claims 15, and 21-24 which exhibits weight percent loss of about 0.6% between about 35° C. to about 220° C. by a thermogravimetric analysis (TGA).

26. The crystalline form of claim 14, wherein the pamoate salt is a solvate.

27. The crystalline form of claim 26, wherein the pamoate salt is an ethanol solvate.

28. The crystalline form of claim 27, wherein the pamoate salt ethanol solvate exhibits an X-ray powder diffraction (XRPD) pattern comprising peaks at about 7.39±0.2 and 26.03±0.2 degrees two-theta.

29. The crystalline form of claim 28, wherein the XRPD pattern further comprises peaks at 10.12±0.2, 21.26±0.2, and 25.70±0.2 degrees two-theta.

30. The crystalline form of any one of claims 27-29 which exhibits an XRPD pattern substantially similar to FIG. 3A.

31. The crystalline form of any one of claims 27-30 which exhibits a differential scanning calorimetry (DSC) thermogram comprising an endotherm peak which onsets at about 238° C.

32. The crystalline form of any one of claims 27-31 which exhibits a differential scanning calorimetry (DSC) thermogram comprising an exotherm peak which onsets at about 170° C.

33. The crystalline form of any one of claims 27-32 which exhibits weight percent loss of about 7.2% between about 35° C. to about 200° C. by a thermogravimetric analysis (TGA).

34. The crystalline form of claim 26, wherein the pamoate salt is an N-methyl pyrrolidone solvate.

35. The crystalline form of claim 34, wherein the pamoate salt N-methyl pyrrolidone solvate exhibits an X-ray powder diffraction (XRPD) pattern comprising peaks at about 7.46±0.2, 20.27±0.2, and 24.93±0.2 degrees two-theta.

36. The crystalline form of claim 35, wherein the XRPD pattern further comprises peaks at 21.05±0.2, 22.44±0.2, and 24.57±0.2 degrees two-theta.

37. The crystalline form of any one of claims 34-36 which exhibits an XRPD pattern substantially similar to FIG. 4A.

38. The crystalline form of any one of claims 34-37 which exhibits a differential scanning calorimetry (DSC) thermogram comprising an endotherm peak which onsets at about 243° C.

39. The crystalline form of any one of claims 34-38 which exhibits weight percent loss of about 33% between about 35° C. to about 140° C. by a thermogravimetric analysis (TGA).

40. The crystalline form of any one of claims 34-39 which exhibits weight percent loss of about 9.5% between about 140° C. to about 220° C. by a thermogravimetric analysis (TGA).

41. The crystalline form of claim 26, wherein the pamoate salt is an N,N-dimethylacetamide solvate.

42. The crystalline form of claim 41, wherein the pamoate salt N,N-dimethylacetamide solvate exhibits an X-ray powder diffraction (XRPD) pattern comprising peaks at about 6.81±0.2, 8.31±0.2, and 24.60±0.2 degrees two-theta.

43. The crystalline form of claim 41 or 42 which exhibits an XRPD pattern substantially similar to FIG. 5.

44. The crystalline form of claim 41, wherein the pamoate salt N,N-dimethylacetamide solvate exhibits an X-ray powder diffraction (XRPD) pattern comprising peaks at about 11.44±0.2, 13.81±0.2, and 24.55±0.2 degrees two-theta.

45. The crystalline form of claim 44, wherein the XRPD pattern further comprises peaks at 6.91±0.2, 12.21±0.2, and 22.70±0.2 degrees two-theta.

46. The crystalline form of any one of claims 41, 44, and 45 which exhibits an XRPD pattern substantially similar to FIG. 6.

47. The crystalline form of claim 41, wherein the pamoate salt N,N-dimethylacetamide solvate exhibits an X-ray powder diffraction (XRPD) pattern comprising peaks at about 18.86 and 23.51 degrees two-theta.

48. The crystalline form of claim 47, wherein the XRPD pattern further comprises peaks at 17.38±0.2, 20.54±0.2, and 21.24±0.2 degrees two-theta.

49. The crystalline form of any one of claims 41, 47, and 48 which exhibits an XRPD pattern substantially similar to FIG. 7.

50. The crystalline form of claim 41, wherein the pamoate salt N,N-dimethylacetamide solvate exhibits an X-ray powder diffraction (XRPD) pattern comprising peaks at about 14.06±0.2 and 21.14±0.2 degrees two-theta.

51. The crystalline form of claim 50, wherein the XRPD pattern further comprises peaks at 19.16±0.2, 21.93±0.2, and 22.36±0.2 degrees two-theta.

52. The crystalline form of any one of claims 41, 50, and 51 which exhibits an XRPD pattern substantially similar to FIG. 8A.

53. The crystalline form of any one of claims 41 and 50-52 which exhibits a differential scanning calorimetry (DSC) thermogram comprising an endotherm peak which onsets at about 247° C.

54. The crystalline form of any one of claims 41 and 50-53 which exhibits weight percent loss of about 55% between about 35° C. to about 97° C. by a thermogravimetric analysis (TGA).

55. The crystalline form of any one of claims 41 and 50-54 which exhibits weight percent loss of about 5.3% between about 97° C. to about 125° C. by a thermogravimetric analysis (TGA).

56. The crystalline form of claim 26, wherein the pamoate salt is a hexafluoroisopropyl acrylate solvate.

57. The crystalline form of claim 56, wherein the pamoate salt hexafluoroisopropyl acrylate solvate exhibits an X-ray powder diffraction (XRPD) pattern comprising peaks at about 4.73±0.2 and 9.45±0.2 degrees two-theta.

58. The crystalline form of claim 56 or 57 which exhibits an XRPD pattern substantially similar to FIG. 9A.

59. The crystalline form of any one of claims 56-58 which exhibits a differential scanning calorimetry (DSC) thermogram comprising an endotherm peak which onsets at about 282° C.

60. The crystalline form of any one of claims 56-59 which exhibits weight percent loss of about 2.3% between about 35° C. to about 80° C. by a thermogravimetric analysis (TGA).

61. The crystalline form of any one of claims 56-60 which exhibits weight percent loss of about 8.0% between about 80° C. to about 200° C. by a thermogravimetric analysis (TGA).

62. The crystalline form of claim 26, wherein the pamoate salt is an N,N-dimethylformamide solvate.

63. The crystalline form of claim 62, wherein the pamoate salt N,N-dimethylformamide solvate exhibits an X-ray powder diffraction (XRPD) pattern comprising peaks at about 5.82±0.2, 11.81±0.2, and 11.62±0.2 degrees two-theta.

64. The crystalline form of claim 63, wherein the XRPD pattern further comprises peaks at 17.46±0.2, 23.49±0.2, and 23.61±0.2 degrees two-theta.

65. The crystalline form of any one of claims 62-64 which exhibits an XRPD pattern substantially similar to FIG. 10A.

66. The crystalline form of any one of claims 62-65 which exhibits a differential scanning calorimetry (DSC) thermogram comprising an endotherm peak which onsets at about 249° C.

67. The crystalline form of any one of claims 62-66 which exhibits weight percent loss of about 0.4% between about 35° C. to about 100° C. by a thermogravimetric analysis (TGA).

68. The crystalline form of any one of claims 62-67 which exhibits weight percent loss of about 9.5% between about 100° C. to about 200° C. by a thermogravimetric analysis (TGA).

69. The crystalline form of any one of claims 14-68, wherein the crystalline form has a purity of about 90% or higher.

70. The crystalline form of any one of claims 14-68, wherein the crystalline form has a purity of about 95% or higher.

71. The crystalline form of any one of claims 14-68, wherein the crystalline form has a purity of about 98% or higher.

72. The crystalline form of any one of claims 14-71, wherein the crystalline form comprises less than 0.5% of bromide ion (Br⁻) by weight.

73. The crystalline form of any one of claims 14-72, wherein the crystalline form is purified and isolated.

74. A pharmaceutical composition comprising any one of the crystalline forms of claims 14-73 and a pharmaceutically acceptable excipient or carrier.

75. A pharmaceutical composition comprising the crystalline form of claim 72 and a pharmaceutically acceptable excipient or carrier.

76. The pharmaceutical composition of any one of claims 12-13 and 74-75, further comprising one or more additional therapeutically active agent.

77. The pharmaceutical composition of claim 76, wherein the one or more additional therapeutically active agent is a cytotoxic agent or an anticancer agent.

78. The pharmaceutical composition of claim 76 or 77, wherein the one or more additional therapeutically active agent is rituximab, docetaxel, carboplatin, paclitaxel, or prednisone.

79. A method of treating cancer comprising administering to a subject in need thereof, an effective amount of any one of the pamoate salt or solvate thereof of claims 1-11, any one of the crystalline form of claims 14-72, or any one of the pharmaceutical composition of claims 12-13 and 74-78.

80. The method of claim 79, wherein the cancer has a c-Myc rearrangement, amplification, or overexpression, and/or MYCN amplification or overexpression.

81. The method of claim 79 or 80, wherein the cancer is a solid tumor or a hematologic malignancy.

82. The method of claim 81, wherein the hematologic malignancy is myeloid disorder, lymphoid disorder, leukemia, lymphoma, myelodysplastic syndrome (MDS), myeloproliferative disease (MPD), mast cell disorder, or myeloma.

83. The method of claim 81, wherein the hematologic malignancy is acute lymphoblastic leukemia (ALL), T-cell ALL (T-ALL), B-cell ALL (B-ALL), acute myeloid leukemia (AML), granulocytic leukemia, monocytic leukemia, lymphocytic leukemia, chronic lymphocytic leukemia (CLL), chronic myelogenous leukemia (CML), blast phase CML, small lymphocytic lymphoma (SLL), CLL/SLL, Hodgkin lymphoma (HL), non-Hodgkin lymphoma (NHL), B-cell NHL, CD20-positive B cell NHL, T-cell NHL, indolent NHL (iNHL), high-grade B-cell lymphoma (HGBCL), diffuse large B-cell lymphoma (DLBCL), mantle cell lymphoma (MCL), aggressive B-cell NHL, B-cell lymphoma (BCL), Richter's syndrome (RS), T-cell lymphoma (TCL), peripheral T-cell lymphoma (PTCL), cutaneous T-cell lymphoma (CTCL), transformed mycosis fungoides, Sezary syndrome, anaplastic large-cell lymphoma (ALCL), follicular lymphoma (FL), Waldenstrom macroglobulinemia (WM), lymphoplasmacytic lymphoma, Burkitt lymphoma, malignant lymphoma, plasmocytoma, reticulum cell sarcoma, multiple myeloma (MM), metastatic myeloma, amyloidosis, MPD, essential thrombocytosis (ET), myelofibrosis (MF), polycythemia vera (PV), chronic myelomonocytic leukemia (CMML), myelodysplastic syndrome (MDS), high-risk MDS, or low-risk MDS.

84. The method of claim 81, wherein the hematologic malignancy is a high-grade B-cell lymphoma (HGBCL) or a diffuse large B-cell lymphoma (DLBCL).

85. The method of claim 79 or 80, wherein the cancer is prostate cancer, breast cancer, lung cancer, brain cancer, liver cancer, uterine cancer, pancreatic cancer, gallbladder cancer, bile duct cancer, colon or rectum cancer, urinary bladder cancer, kidney cancer, ovarian cancer, esophageal cancer, gastric (stomach) cancer, head and neck cancer, bone cancer, thyroid cancer, cervical cancer, lymphoma, or leukemia.

86. The method of claim 79 or 80, wherein the cancer is small cell lung cancer, non-small cell lung cancer (NSCLC), neuroendocrine prostate cancer (NEPC), hormone refractory prostate cancer (HRPC), neuroblastoma, triple negative breast cancer (TNBC), HER2 negative metastatic breast cancer, or hemangiosarcoma.

87. The method any one of claims 79-86, wherein the cancer is relapsed or refractory.

88. The method of any one of claims 79-86, further comprising administering one or more additional therapeutically active agent.

89. The method composition of claim 88, wherein the one or more additional therapeutically active agent is a cytotoxic agent or an anticancer agent.

90. The method of claim 88 or 89, wherein the one or more additional therapeutically active agent is rituximab, docetaxel, carboplatin, paclitaxel, or prednisone.

91. A method of inhibiting or reducing the expression of c-Myc protein level in a cancer cell of a subject, comprising administering to the subject, any one of the pamoate salt or solvate thereof of claims 1-11, any one of the crystalline form of claims 14-72, or any one of the pharmaceutical composition of claims 12-13 and 74-78.

92. A method of degrading c-Myc protein in a cancer cell of a subject, comprising administering to the subject, any one of the pamoate salt or solvate thereof of claims 1-11, any one of the crystalline form of claims 14-72, or any one of the pharmaceutical composition of claims 12-13 and 74-78.

93. The method of any one of claims 79-92, wherein the pamoate salt or solvate thereof, the crystalline form, or the pharmaceutical composition is administered to the subject orally, nasally, transdermally, topically, pulmonary, inhalationally, buccally, sublingually, intraperintoneally, subcutaneously, intramuscularly, intravenously, rectally, intrapleurally, intrathecally, intraportally, or parenterally, or as an implant.

94. A pharmaceutical composition of any one of claims 74-78, wherein the composition is formulated to be administered orally, nasally, transdermally, topically, pulmonary, inhalationally, buccally, sublingually, intraperintoneally, subcutaneously, intramuscularly, intravenously, rectally, intrapleurally, intrathecally, intraportally, or parenterally, or as an implant.
